(19) United States
(12) Patent Application Publication
(10) Pub. No.: US 2026/0155261 A1
(43) Pub. Date: Jun. 4, 2026

(54) BROAD-SPECTRUM NATIONAL BIODEFENSE STRATEGY BASED UPON EPIGENETIC DEFENSE

(71) Applicant: Channel Content Company LLC, Sheridan, WY (US)

(72) Inventor: Jeff Gusky, Holly Lake Ranch, TX (US)

(21) Appl. No.: 19/460,283

(22) Filed: Jan. 26, 2026

Related U.S. Application Data
(63) Continuation-in-part of application No. PCT/US24/40097, filed on Jul. 29, 2024.
Application No. PCT/US24/40097 is a continuation-in-part of application No. 18/227,919, filed on Jul. 29, 2023, now Pat. No. 12,100,497.
Application No. 18/227,919 is a continuation-in-part of application No. 18/227,925, filed on Jul. 29, 2023, now Pat. No. 12,117,189.
Application No. 18/227,925 is a continuation-in-part of application No. 18/227,930, filed on Jul. 29, 2023, now Pat. No. 12,075,803.

(51) Int. Cl. G16H 50/30; A61K 31/197; A61K 31/593; A61K 31/7105; A61K 35/741; A61K 45/06; G01N 33/68; G01N 33/82; G16H 15/00; G16H 20/10; G16H 40/67; G16H 50/70

(52) U.S. Cl. CPC G16H 50/30; A61K 31/197; A61K 31/593; A61K 31/7105; A61K 35/741; A61K 45/06; G01N 33/6815; G01N 33/82; G16H 15/00; G16H 20/10; G16H 40/67; G16H 50/70

(57) ABSTRACT

Disclosed are non-pharmaceutical compositions, kits, systems, and methods configured to support immunological fitness and continuous cellular defenses. In certain embodiments, a kit includes a bioavailability-optimized ingestible powder and a bioavailability-optimized ingestible liquid provided together with guidance associating coordinated use of the compositions with activation of one or more biological defense pathways. Additional embodiments include preparedness kits comprising interim vitamin D dosing components and guidance materials suitable for deployment prior to individualized testing. System embodiments may integrate distribution mechanisms, data association components, processing components, and optional environmental sensing to support population-scale risk mitigation. Methods are disclosed for supporting immunological fitness, maintaining biological barriers, and associating measurable biological and environmental metrics with population-level risk assessment and actuarial applications, without requiring pharmaceutical intervention.

### CROSS-REFERENCE TO RELATED APPLICATIONS

This application is a continuation in part of International Application No. PCT/US 2024/040097 filed on Jul. 29, 2024, the entire content of which is incorporated herein by reference. The International application No. PCT/US2024/040097 is a Continuation in Part of the following US Patent Applications: The U.S. patent application Ser. No. 18/227,919, filed on Jul. 29, 2023, U.S. patent application Ser. No. 18/227,925, filed on Jul. 29, 2023, and U.S. patent application Ser. No. 18/227,930, filed on Jul. 29, 2023, the entire content of each patent is incorporated herein by reference.

This application is a Continuation in Part of provisional patent Application No. 63/698,040 filed on Sep. 23, 2024. The entire content of which is incorporated herein by reference.

### BACKGROUND

The present invention relates to the induction and maintenance of population-scale immune defenses against epigenetic warfare.

### SUMMARY

According to an illustrative embodiment of the invention there is a non-pharmaceutical immunological fitness and continuous cellular defenses kit. The kit comprises a bioavailability optimized ingestible powder comprising prebiotics, probiotics, postbiotics, essential micronutrients, bioavailability optimized phytochemicals, microRNAs, and amino acids; a bioavailability optimized ingestible liquid comprising non-pharmaceutical phytochemicals and essential micronutrients, wherein the non-pharmaceutical phytochemicals are encapsulated using food-grade nanotechnology to improve bioavailability; and instructions provided with the kit associating coordinated use of the ingestible powder and the ingestible liquid with activation of at least one biological defense pathway selected from a vitamin D and homocysteine pathway, an immunological fitness pathway, and a continuous cellular defenses pathway and wherein the kit, as packaged and distributed, is configured for population-scale deployment to mitigate immune dysregulation and epigenetic injury without requiring pharmaceutical intervention.

According to an illustrative embodiment, there is a non-pharmaceutical immunological preparedness kit, comprising: a plurality of ingestible compositions packaged together, including at least one bioavailability optimized powder and at least one bioavailability optimized liquid; an interim vitamin D dosage component packaged for administration prior to individualized serum vitamin D testing; and guidance materials provided with the kit associating use of the ingestible compositions with immunological fitness qualification metrics and wherein the kit is configured for population-scale preparedness against immune dysregulation independent of user-performed medical treatment.

According to an illustrative embodiment there is a system for population-scale immunological risk mitigation. The system comprises a plurality of non-pharmaceutical immunological fitness kits, each kit comprising at least one ingestible powder and at least one ingestible liquid configured to support immunological fitness; a distribution mechanism configured to deliver the plurality of kits to a population group; a data association component configured to associate kit distribution or usage with immunological fitness metrics; and a processing component configured to determine compliance status, qualification status, or population-level risk metrics based on the immunological fitness metrics.

According to an illustrative embodiment, there is a method for immunological fitness and continuous cellular defenses, comprising: activating at least one of four pathways to induce components of immunological fitness and continuous cellular defenses in a plurality of people, where the four pathways comprise a vitamin D and homocysteine pathway, an indoor viral respiratory risk mitigation pathway, an immunological fitness pathway, and a continuous cellular defenses pathway. The method achieves zero-order pharmacokinetics of vitamin D and maintains serum homocysteine below a predetermined threshold. The method optimizes biological barriers against airborne pathogens through maintenance of viral-safe indoor absolute humidity. The method administers non-pharmaceutical nutritional compositions to induce immunological fitness and continuous cellular defenses. The method mitigates a risk of immune dysregulation, cytokine storm, and epigenetic injury based on activation of the at least one pathway.

In an illustrative embodiment, there is a method for lowering insurance underwriting risk for a plurality of people. The method deploys one or more non-pharmaceutical immunological fitness kits to the plurality of people. The method measures quantifiable biological outcomes including serum vitamin D level, serum homocysteine level, and biological barrier optimization. The method compares measured biological outcomes to actuarial risk benchmarks. The method validates compliance through an independent assessment. The method generates and provides underwriting-relevant risk reduction data or rate-adjustment recommendations to one or more third-party insurance underwriters based on validated reduction data.

The foregoing is a summary and thus contains, by necessity, simplifications, generalizations, and omissions of detail; consequently, those skilled in the art will appreciate that the summary is illustrative only and is not intended to be in any way limiting. Other aspects, inventive features, and advantages of the present invention will be apparent in the non-limiting detailed description set forth below.

### BRIEF DESCRIPTION OF THE DRAWINGS

The present invention may be better understood, and its numerous objects, features, and advantages made apparent to those skilled in the art by referencing the accompanying drawings, wherein:

FIG. 1 depicts a process that activates immunological fitness and continuous cellular defenses;

FIG. 2 depicts an absolute humidity safety process;

FIG. 3 depicts a process that activates cell-mediated immunity (CMI) on mucosal surfaces in a respiratory tract of an individual;

FIG. 4 depicts a process that indicates viral safety of enclosed spaces;

FIG. 5 depicts a first action process taken when viral safety is not safe;

FIG. 6 depicts a schematic view of an indoor safety viewers (ISV);

FIG. 7 depicts an indoor safety indication process;

FIG. 8 depicts a schematic view of a computer network cloud environment;

FIG. 9 depicts a schematic view of a HIPAA compliant interactive communication system overview;

FIG. 10 depicts a schematic diagram of a system having support for a sensor directory;

FIG. 11 depicts a list of example sensor APIs;

FIG. 12 depicts an embodiment enabling an unsafe indication on a mobile device;

FIG. 13 depicts a process to perform mass testing and treatment on a plurality of people to elevate a serum vitamin D level into a viral safe range;

FIG. 14 depicts a process that facilitates activation of gut microbiome Cell-mediated Herd Immunity®¹ (CMHI) in a group of people; ¹ CELL-MEDIATED HERD IMMUNITY is a registered trademark of Jeff Gusky.

FIG. 15 depicts a process for restoring and maintaining a healthful gut microbiome;

FIG. 16 depicts a process for facilitating activation CMHI by activating CMI;

FIG. 17 depicts process to induce a continuous antiviral state in a plurality of people;

FIG. 18 depicts a process to assess spike protein induced thromboembolism risk in an individual;

FIG. 19 depicts a process to provide immediate treatment of a person with initial viral symptoms by rapidly activating individual CMI;

FIG. 20 depicts a process for rapid induction of CMI in new people joining a group;

FIG. 21 depicts a process for assessing and mitigating risk of spike protein induced immune suppression and endothelial inflammation;

FIG. 22 depicts a process taken by a company-specific viral safety certification program;

FIG. 23 depicts a process taken by a process that recruits, credentials, onboards, and trains qualified service providers supporting CMHI for community health;

FIG. 24 depicts a process taken to reduce company insurance premiums;

FIG. 25 depicts a high-level schematic representation of an intelligent system designed for facilitating contextually relevant conversational interactions;

FIG. 26 depicts process of achieving saturated and functional serum vitamin D and safe serum homocysteine;

FIG. 27 depicts a high-level block diagram representation of an AI system;

FIG. 28 depicts an embodiment of an Artificial Intelligence (AI) system trained to support bulk testing;

FIG. 29 depicts a process taken by a user registration process;

FIG. 30 depicts a representation of a user profile data which may include user identification and demography;

FIG. 31 depicts a high-level representation of user specific data;

FIG. 32 depicts a schematic view of an Artificial Intelligence (AI) event booking infrastructure;

FIG. 33 depicts a process for dynamically facilitating CMHI;

FIG. 34 depicts a process for event booking success prediction model;

FIG. 35 depicts an embodiment of a population-scale immune defense package;

FIG. 36 depicts an embodiment of population-scale immune defense process details;

FIG. 37 depicts a process for an immediate treatment kit details;

FIG. 38 depicts a process for immunological fitness details;

FIG. 39 depicts an embodiment of a nutritional warfare package;

FIG. 40 depicts a nutritional warfare process details;

FIG. 41 depicts a process for target group compliance assessment;

FIG. 42 depicts examples of determining expected outcome metrics;

FIG. 43 depicts a process for qualifying for preferred risk groups;

FIG. 44 depicts a process for qualifying a plurality of people for insurance discounts;

FIG. 45 depicts a process for providing tiered insurance discounts;

FIG. 46 depicts a non-pharmaceutical immunological fitness and continuous cellular defenses kit;

FIG. 47 depicts embodiments of non-pharmaceutical immunological fitness and continuous cellular defenses kit;

FIG. 48 depicts a non-pharmaceutical immunological preparedness kit;

FIG. 49 depicts embodiments of non-pharmaceutical immunological fitness and continuous cellular defenses kit;

FIG. 50 depicts a system for population-scale immunological risk mitigation;

FIG. 51 depicts embodiments of the system for population-scale immunological risk mitigation;

FIG. 52 depicts a flow diagram of a method for immunological fitness and continuous cellular defenses;

FIG. 53 depicts embodiments of the method for immunological fitness and continuous cellular defenses;

FIG. 54 depicts a flow diagram of a method for lowering insurance underwriting risk for a plurality of people;

FIG. 55 depicts an embodiment of an immediate treatment kit;

FIG. 56 depicts a process of deploying a plurality of the four separate pathways as biodefense countermeasures at population scale;

FIG. 57 depicts combinations of pathways to mitigate biodefense risks;

FIG. 58 depicts an embodiment of the vitamin D and homocysteine pathway;

FIG. 59 depicts a process of implementing the indoor viral respiratory risk mitigation pathway;

FIG. 60 depicts a process for receiving real-time absolute humidity data from a plurality of absolute humidity sensors;

FIG. 61 depicts a process for outputting the received real-time absolute humidity data to visual displays;

FIG. 62 depicts a process for providing an ability to remotely assess the indoor viral respiratory pandemic risk;

FIG. 63 depicts a process for receiving the data from the database for an actuarial assessment for insurance assessment input;

FIG. 64 depicts a process for implementing the immunological fitness pathway by providing a non-pharmaceutical immunological fitness emergency treatment kit;

FIG. 65 depicts a process for implementing the continuous cellular defenses pathway by providing a non-pharmaceutical continuous cellular defenses pathway emergency treatment kit;

FIG. 66 depicts a process for enabling a state of immunological fitness in the plurality of people by optimizing CMI;

FIG. 67 depicts a process for enabling a state of continuous cellular defenses in the plurality of people by simultaneously optimizing a multitude of intrinsic cellular defense mechanisms against epigenetic attack by pathogens;

FIG. 68 depicts a process for augmenting rapid cellular sensing and recognition of pathogens, preventing immune evasion by the pathogens and by activating host defense peptides which degrade pathogens on contact;

FIG. 69 depicts a process for mitigating risk of long term epigenetic injuries;

FIG. 70 depicts a process for optimizing biological barriers via physical interventions on a surface of the biological barrier;

FIG. 71 depicts a process for creating within the plurality of people a continuous antiviral, antibacterial, antifungal, antiparasitic homeostasis that shields human cells against epigenetic injuries;

FIG. 72 depicts a process for inducing a continuous state of zero-order pharmacokinetics of vitamin D;

FIG. 73 depicts a process for reducing approximately 40% incidence of vitamin D low responders;

FIG. 74 depicts a process for achieving in the plurality of people a homeostasis of continuous antioxidation, antiinflammation, immunomodulation, antiviral, antibacterial, antifungal, antiparasitic priming, restoring metabolic switching, metabolic signaling, and bioenergetic priming;

FIG. 75 depicts a process for providing periodic mass testing of serum vitamin D, serum homocysteine, and serum calcium;

FIG. 76 depicts a process for inducing epigenetic rewiring which transforms harmful epigenetic memory to healthful epigenetic memory and stimulates trained innate immunity;

FIG. 77 depicts a process for preserving a state of continuous trained innate immunity and providing continuous epigenetic defense against new episodes of harmful epigenetic reprogramming;

FIG. 78 depicts a process for mitigating symptoms of chronic diseases;

FIG. 79 depicts a process for lowering insurance underwriting rates for a plurality of people by lowering risks associated with harmful epigenetic injuries;

FIG. 80 depicts a process for providing a supplement protocol for mitigating additional insurance underwriting risks;

FIG. 81 depicts a process for lowering insurance rates by testing and treatment of harmful epigenetic injuries;

FIG. 82 depicts a process for providing insurance discounts based on measuring, qualifying, and reporting quantifiable measures which activate immunological fitness;

FIG. 83 depicts a process for maintaining the immunological fitness pathway by providing a non-pharmaceutical immunological fitness maintenance treatment kit;

FIG. 84 depicts a process for maintaining the continuous cellular defenses pathway by providing a non-pharmaceutical continuous cellular defenses maintenance treatment kit;

FIG. 85 depicts a process for qualifying a plurality of people for an absolute humidity discount;

FIG. 86 depicts a process for implementing the vitamin D and homocysteine pathway by providing mass testing and treatment;

FIG. 87 depicts a process for lowering risks associated with harmful epigenetic injuries customized to group characteristic;

FIG. 88 depicts a process for lowering insurance rates for a new plurality of people by lowering risks associated with harmful epigenetic injuries;

FIG. 89 depicts a schematic view of a processing system wherein the methods of this invention may be implemented.

In the figures, boxes connected by dotted lines represent informational, material, or functional inputs into a process step and do not represent discrete process steps unless connected by a solid arrow.

### DETAILED DESCRIPTION

According to an illustrative embodiment of the invention there is a non-pharmaceutical immunological fitness and continuous cellular defenses kit. The kit comprises a bioavailability optimized ingestible powder comprising prebiotics, probiotics, postbiotics, essential micronutrients, bioavailability optimized phytochemicals, microRNAs, and amino acids; a bioavailability optimized ingestible liquid comprising non-pharmaceutical phytochemicals and essential micronutrients, wherein the non-pharmaceutical phytochemicals are encapsulated using food-grade nanotechnology to improve bioavailability; and instructions provided with the kit associating coordinated use of the ingestible powder and the ingestible liquid with activation of at least one biological defense pathway selected from a vitamin D and homocysteine pathway, an immunological fitness pathway, and a continuous cellular defenses pathway and wherein the kit, as packaged and distributed, is configured for population-scale deployment to mitigate immune dysregulation and epigenetic injury without requiring pharmaceutical intervention. In an embodiment the kit has an immediate treatment kit component configured for short-term use in response to early signs of infectious illness. In an embodiment the kit is configured as a maintenance kit comprising a supply of the ingestible powder and the ingestible liquid for repeated daily use over a predetermined maintenance period. In an embodiment, the ingestible powder and the ingestible liquid are formulated according to demographic classifications selected from adults, children, military personnel, first responders, or institutional populations. In an embodiment, components of the kit are functionally interdependent and lack substantial non-infringing use when distributed together. In an embodiment, the instructions, labeling, or electronic content expressly direct coordinated use of the ingestible powder and the ingestible liquid to activate the at least one biological defense pathway. In an embodiment, the kit is marketed, labeled, or configured to mitigate actuarial risk associated with immune dysregulation and epigenetic injury. In an embodiment, removal or substitution of any one component materially degrades an intended immunological risk-mitigation functionality of the kit.

In various embodiments, the non-pharmaceutical immunological fitness and continuous cellular defenses kit provides the technical advantage of enabling coordinated activation of multiple biological defense pathways through packaged, non-pharmaceutical ingestible compositions, without requiring pharmaceutical agents, physician-directed treatment, or individualized medical intervention. The kit architecture advantageously supports population-scale deployment by enabling standardized distribution, simplified instructions, and coordinated use of functionally interdependent components, thereby improving consistency of use, compliance, and scalability across heterogeneous populations. The kit further enables synergistic biological effects arising from coordinated administration of complementary ingestible compositions, while reducing reliance on single-mechanism interventions.

According to an illustrative embodiment, there is a non-pharmaceutical immunological preparedness kit, comprising: a plurality of ingestible compositions packaged together, including at least one bioavailability optimized powder and at least one bioavailability optimized liquid; an interim vitamin D dosage component packaged for administration prior to individualized serum vitamin D testing; and guidance materials provided with the kit associating use of the ingestible compositions with immunological fitness qualification metrics and wherein the kit is configured for population-scale preparedness against immune dysregulation independent of user-performed medical treatment. The interim vitamin D dosage component is configured for administration for a predetermined duration without serum vitamin D testing. The guidance materials may associate kit usage with target serum vitamin D and serum homocysteine ranges. The ingestible compositions may be packaged for distribution to institutional, occupational, or community populations. The plurality of ingestible compositions may be structured and promoted as a unified immunological defense solution. In an embodiment, removal or substitution of any one of the ingestible compositions may materially degrades an intended immunological risk-mitigation functionality of the kit.

In various embodiments, the non-pharmaceutical immunological preparedness kit provides the advantage of enabling pre-deployment immunological readiness in advance of individualized testing or medical evaluation. The preparedness kit reduces time-to-readiness by supplying interim nutritional components configured for safe, short-term use, thereby facilitating early risk mitigation during periods of heightened biological risk. This configuration further enables rapid, population-level preparedness independent of clinical bottlenecks, while maintaining compatibility with later individualized testing, refinement, or stratification without requiring modification of the kit architecture.

According to an illustrative embodiment there is a system for population-scale immunological risk mitigation. The system comprises a plurality of non-pharmaceutical immunological fitness kits, each kit comprising at least one ingestible powder and at least one ingestible liquid configured to support immunological fitness; a distribution mechanism configured to deliver the plurality of kits to a population group; a data association component configured to associate kit distribution or usage with immunological fitness metrics; and a processing component configured to determine compliance status, qualification status, or population-level risk metrics based on the immunological fitness metrics. The system may include a plurality of environmental sensors configured to measure indoor absolute humidity and associate measured indoor absolute humidity values with biological barrier optimization metrics. Deployment of the kits without the data association component may materially reduces an intended population-scale immunological risk-mitigation benefit. The system is offered, licensed, or deployed with knowledge that distribution of the kits will result in use consistent with predefined immunological defense pathways.

In various embodiments, the system for population-scale immunological risk mitigation provides the advantage of integrating physical distribution of non-pharmaceutical kits with data association and processing components that enable population-level assessment without requiring centralized medical control. The system architecture advantageously supports scalable monitoring, qualification, and compliance evaluation by associating kit usage with quantifiable immunological fitness metrics. This integration enables adaptive population-level risk management, supports decentralized deployment, and facilitates interoperability with third-party analytical, administrative, or actuarial systems.

According to an illustrative embodiment, there is a method for immunological fitness and continuous cellular defenses, comprising: activating at least one of four pathways to induce components of immunological fitness and continuous cellular defenses in a plurality of people, wherein the four pathways comprise a vitamin D and homocysteine pathway, an indoor viral respiratory risk mitigation pathway, an immunological fitness pathway, and a continuous cellular defenses pathway; achieving zero-order pharmacokinetics of vitamin D and maintaining serum homocysteine below a predetermined threshold; optimizing biological barriers against airborne pathogens through maintenance of viral-safe indoor absolute humidity; administering non-pharmaceutical nutritional compositions to induce immunological fitness and continuous cellular defenses; and mitigating risk of immune dysregulation, cytokine storm, and epigenetic injury based on activation of the at least one pathway. In an embodiment, administering non-pharmaceutical nutritional compositions comprises coordinated administration of a bioavailability optimized powder and a bioavailability optimized liquid. In an embodiment, determining vitamin D dosing may be based on serum testing results. In an embodiment, vitamin D is administered for a predetermined interim period without serum testing. In an embodiment, inducing epigenetic rewiring may transform harmful epigenetic memory into trained innate immunity.

In various embodiments, the method for immunological fitness and continuous cellular defenses provides the advantage of enabling coordinated activation of multiple independent defense pathways using non-pharmaceutical interventions, thereby reducing vulnerability to single-point failure mechanisms. The method advantageously integrates nutritional compositions and environmental optimization to support biological barrier integrity and systemic defense readiness without requiring diagnostic confirmation of infection or pathogen identification. This approach improves robustness, reduces latency between exposure risk and protective activation, and supports continuous baseline readiness across diverse environments.

In an illustrative embodiment, there is a method for lowering insurance underwriting risk for a plurality of people. The method deploys one or more non-pharmaceutical immunological fitness kits to the plurality of people; measures quantifiable biological outcomes including serum vitamin D level, serum homocysteine level, and biological barrier optimization. Compares measured biological outcomes to actuarial risk benchmarks. Validates compliance through an independent assessment; and adjusts insurance underwriting rates based on validated reductions in biological risk. The method may form a preferred insurance risk pool comprising populations demonstrating sustained compliance with immunological fitness protocols. The method of may assign tiered insurance discounts based on graduated levels of immunological fitness and indoor environmental safety.

In an illustrative embodiment, there is a method for lowering insurance underwriting risk for a plurality of people. The method deploys one or more non-pharmaceutical immunological fitness kits to the plurality of people; measures quantifiable biological outcomes including serum vitamin D level, serum homocysteine level, and biological barrier optimization. The method compares measured biological outcomes to actuarial risk benchmarks. The method validates compliance through an independent assessment. The method generates and provides underwriting-relevant risk reduction data or rate-adjustment recommendations to one or more third-party insurance underwriters based on validated reduction data. The underwriting-relevant data comprises a recommended premium modifier, risk class adjustment, or eligibility qualification. The method may electronically interfacing with a third-party underwriting system to transmit the validated biological risk reduction data. The underwriting-relevant data may be updated dynamically based on subsequent biological outcome measurements.

In various embodiments, the method for lowering insurance underwriting risk provides the advantage of enabling objective, quantifiable linkage between biological and environmental risk-mitigation measures and actuarial assessment frameworks. By associating non-pharmaceutical interventions with measurable outcomes, the method enables alternative risk modeling approaches that supplement or replace retrospective actuarial data. This facilitates dynamic risk adjustment, incentivizes proactive population-level compliance, and enables insurance systems to account for real-time biological and environmental risk mitigation rather than solely historical loss experience.

According to an illustrative embodiment of the invention, there is a method for immunological fitness and continuous cellular defenses. The method activates at least one of four separate pathways to induce components of immunological fitness and continuous cellular defenses in a plurality of people. The four separate pathways include: vitamin D and homocysteine pathway, indoor viral respiratory risk mitigation pathway, immunological fitness pathway, and continuous cellular defenses pathway in the plurality of people. The vitamin D and homocysteine pathway is activated by achieving zero-order pharmacokinetics for vitamin D and a targeted level of homocysteine below a predetermined level by testing and treatment of vitamin D and homocysteine. The indoor viral respiratory risk mitigation pathway is activated by optimizing protections provided by biological barriers against infections by airborne pathogens through establishing viral safe indoor absolute humidity and by executing an indoor viral respiratory pandemic risk assessment and risk mitigation. The immunological fitness pathway is activated by mitigating a risk of immune dysregulation and associated risk of cytokine storm in the plurality of people by providing a population-scale immune defense package tailored to induce immunological fitness where the contents of the population-scale immune defense package include: a bioavailability optimized daily ingestible powder where the bioavailability optimized daily ingestible powder has: prebiotics, probiotics, postbiotics, essential micronutrients, bioavailability optimized phytochemicals, microRNAs, and amino acids to be ingested. Immunological fitness is induced responsive to ingesting, by the plurality of people in need thereof. The continuous cellular defenses pathway is activated by mitigating epigenetic injuries in the plurality of people by providing a nutritional warfare package tailored to induce immunological fitness and continuous cellular defenses where the nutritional warfare package includes: a bioavailability optimized daily ingestible liquid including: non-pharmaceutical phytochemicals and essential micronutrients where the non-pharmaceutical phytochemicals in the liquid are encapsulated using food grade nanotechnology to improve their bioavailability and a bioavailability optimized daily ingestible powder where the powder includes: prebiotics, probiotics, postbiotics, essential micronutrients, bioavailability optimized phytochemicals, microRNAs, and amino acids. Immunological fitness and continuous cellular defenses are induced responsive to ingesting the liquid and the powder, by the plurality of people in need thereof. The risks of immune dysregulation and associated risk of cytokine storm, and epigenetic injuries are mitigated based on the activating of the at least one of four separate pathways.

According to an embodiment of the invention, there is a method to lower insurance rates for plurality of people by lowering risk in the plurality of people associated with harmful epigenetic injuries. One or more tailored protocols are identified that are customized to group characteristics of each plurality of people that counteract harmful epigenetic injuries in each plurality of people. Quantifiable outcomes are identified that are inextricably linked to compliance with the tailored protocols customized for each plurality of people. In each plurality of people a pre-protocol dataset of the quantifiable outcomes is established for the plurality of people for a retrospective period of time prior to initiation of the tailored protocol to establish a historical pre-protocol dataset. A post-protocol dataset of the quantifiable outcomes is established in each plurality of people that is inextricably linked to compliance with the tailored protocol for the population for a predetermined period of time. The post-protocol dataset is compared to the pre-protocol dataset to establish a compliance measure for the plurality of people. Responsive to determining the compliance measure for the plurality of people exceeds a predetermined expected improvement, the pre-protocol dataset and the post-protocol dataset are forwarded to a professional independent auditor for validation. Responsive to receiving, from the professional independent auditor, a written confirmation of validity of the pre-protocol dataset and the post-protocol data set, the plurality of people is added to an initial pool of plurality of people with validated post-protocol compliance. Responsive to detecting a size of the initial pool of plurality of people becoming large enough to qualify members of the initial pool of plurality of people with validated post-protocol compliance for lower insurance rates, designating the initial pool of plurality of people with validated post-protocol compliance as a preferred aggregate risk pool. The plurality of people is decoupled from a current aggregate risk pool which is a principal source of actuarial data used to price an insurance product. The plurality of people is recoupled to the preferred aggregate risk pool as the source of validated actuarial risk data on which the insurance rates for the preferred aggregate risk pool are calculated.

According to an embodiment of the invention, there is a method for providing insurance discounts based on measuring, qualifying, and reporting quantifiable measures which activate immunological fitness. A sign-up for the service is from a requestor for one or more recipients. A written consent is received from the one or more recipients allowing communication with insurance companies regarding qualifications for the insurance discounts via Health Insurance Portability and Accountability Act (HIPAA) compliant communication. A first set of products is provided to activate the immunological fitness in the one or more recipients where usage of the products induces quantifiable measures of activated immunological fitness: serum vitamin D level, serum homocysteine level, and gut microbiome analysis in the plurality of recipients. A second set of products is provided to optimize biological barriers via physical intervention on a surface of the biological barriers caused by safe levels of indoor absolute humidity where the plurality of recipients reside. An immunological fitness score is calculated to qualify the one or more recipients for an identified insurance discount where the immunological fitness score is based upon three tiers of the immunological fitness and an indoor absolute humidity discount. A first tier of immunological fitness score is achieved based upon a confirmed serum vitamin D level of 65 ng/mL to 100 ng/mL and a serum homocysteine level less than 11.5 μmol/L in adult men and less than 10.5 μmol/L in adult women; A second tier of immunological fitness score is achieved based upon a confirmed serum vitamin D level of 85 ng/mL to 100 ng/mL and a serum homocysteine level less than 11.5 μmol/L in adult men and less than 10.5 μmol/L in adult women wherein the second tier has a greater discount than the first tier. A third tier of immunological fitness score is achieved based upon a confirmed serum vitamin D level of 85 ng/mL to 100 ng/mL and a serum homocysteine level less than 11.5 μmol/Lin adult men and less than 10.5 μmol/L in adult women and a confirmed gut microbiome analysis showing a healthful gut microbiome wherein the third tier has a greater discount than the second tier. An indoor absolute humidity discount is achieved when the indoor absolute humidity values are maintained at least 10 g/m³ and below approximately 12 g/m³ while HVAC system is not in standby mode. Responsive to a recipient in the one or more recipients achieving a qualification for the identified insurance discount, reporting to the recipient and to the insurance companies the achievement of the qualification for the identified insurance discount.

Disclosed is a method about population-scale risk mitigation of widespread ongoing epigenetic injuries and existential threats to the US military and American cities caused by a new stealth weapons technology against which highly vaccinated populations (with the Covid MRNA vaccines) are presently undefended and about which these populations are presently unaware.

The immune system is the new battlefield of modern warfare. Infliction of population-scale injuries to the immunity of millions is a new way to vanquish modern nations.

Across embodiments, the disclosed compositions, kits, systems, and methods collectively provide the advantage of enabling non-pharmaceutical, population-scale biological risk mitigation that is modular, scalable, and compatible with diverse regulatory, institutional, and operational environments. The disclosed architecture reduces dependency on individualized medical intervention, improves deployment speed, and supports continuous readiness against biological stressors through coordinated nutritional and environmental mechanisms. These advantages are particularly beneficial in large-scale, distributed, or resource-constrained settings.

A new field of science has emerged over the last three decades from the technologies that enable the creation of lab engineered pathogens. This new field is called synthetic biology. A new type of warfare has emerged based on synthetic biology referenced herein as epigenetic warfare.

Epigenetic warfare represents the weaponization of nature. For thousands of years, natural pathogens have attacked human cells by inducing harmful epigenetic reprogramming. For many years, the scientific literature has characterized this induction of harmful epigenetic reprogramming by pathogens as molecular warfare. Molecular warfare involves simultaneous attacks at a molecular level by pathogens against human cells which attempt to override cellular defense mechanisms which protect human cells against pathogens.

Via molecular warfare, pathogens evade detection by (1) interrupting cellular sensing of pathogens; (2) by blocking the release of chemicals produced by human immune cells which destroy pathogens; (3) by impairing immune cells such as phagocytes and natural killer cells which almost instantaneously engage in killing and clearing invading pathogens; (4) by down regulating the activation of genes which produce cytokines like type 1 interferon which trigger cellular signaling cascades that coordinate downstream cellular defenses such as activating specialized genes and up regulating immune cells which kill pathogens.

As opposed to gain-of-function research which studies how to make pathogens more contagious, an established area of scientific research called loss-of-function research studies how pathogens use harmful epigenetic reprogramming to cause loss-of-function of the intrinsic cellular defense mechanisms which protect cells against pathogens.

Epigenetic warfare represents the deployment of a new class of high-tech weapons born of advanced bioengineering technology which enables the creation of lab engineered pathogens that amplify by many fold the harmful epigenetic reprogramming carried out by natural pathogens. While these pathogens could be viral, bacterial, fungal or parasites, for teaching purposes, viruses, in particular, Covid is used herein to exemplify how immunological fitness and continuous cellular defenses can be activated as broad-spectrum protection against all types of pathogens. Covid possesses characteristics consistent with an epigenetic weapon.

The result is super pathogens never-before-seen in human history. These super pathogens are biological weapons which possess the ability to produce harmful epigenetic reprogramming that cause loss-of-function of cellular defense mechanisms that is potentially far more destructive to human cells than molecular warfare carried out by natural pathogens.

Harmful epigenetic reprogramming can alter the human host for a lifetime by producing lasting epigenetic injuries which are heritable from one generation of cells to the next. Epigenetic weapons can be manufactured which the immune systems of entire populations have never seen before. These populations are immunologically naive like newborns whose immune systems have never been exposed to pathogens before. The world was immunologically naive to Covid. When entire populations are immunologically naive, the risk of mass death caused by immune dysregulation which triggers cytokine storm rises substantially.

Epigenetic bioweapons are stealth weapons because an attack can't be felt or seen while the attack is taking place. The explanation for this is that epigenetic bioweapons attack tissues in the human body which have no attachment to sensory nerves. Endothelial cells found in every organ in the body, brain cells and beneficial bacteria in the gut which are crucial to immune function are all attacked by Covid but the attacks are imperceptible to the host.

Epigenetic warfare is asymmetric because even the most powerful militaries cannot defend cities against epigenetic weapons. Prior to the methods disclosed herein, militaries are unable to defend themselves against epigenetic weapons.

Epigenetic attacks occur without warning. Militaries may never know what's hit them until it's too late. And the perpetrators of the attack may remain anonymous. The technologies behind epigenetic warfare are here to stay.

An unrecognized national security threat of population-scale immune dysfunction has existed since long before Covid. The same population-scale impairment of chromatin inducibility which causes population-scale vulnerability to bioweapons also causes population-scale vulnerability to autism, Alzheimer's, atherosclerotic heart disease, type II diabetes, obesity and more. Impairment of chromatin inducibility underlies the rapidly accelerating epidemic of chronic diseases.

The rapidly accelerating chronic disease epidemic can not be reversed merely by promotion of healthy lifestyles or by new regulations which limit toxic compounds in the foods we eat or in the environment. Without population-scale immunological fitness, the chronic disease epidemic will never end because population-scale impairment of chromatin inducibility is the root cause of the problem. Defending the nation against the existential threat of epigenetic warfare will simultaneously reverse the chronic disease epidemic with no additional cost. Open chromatin is known as euchromatin. Closed chromatin is called heterochromatin. The epigenome controls the opening and closing of chromatin. About 90% of a cell's chromatin is closed at any given time. When cells can't convert heterochromatin to euchromatin, they can't transcribe DNA which is how cells manufacture proteins and other life-critical molecules.

Pathologic epigenetic reprogramming induced by pathogens, cancers and many environmental toxins make CMI dysfunctional by inducing immune tolerance which down regulates gene activation by making the epigenome less responsive to innate immune signaling. This down regulation of the immune responsiveness to pathogens, cancer, environmental toxins and many different types of inflammatory conditions is referred to as gene silencing. Gene silencing is a double-edged sword: while it is a vital component of innate immune defense and tolerance, its dysregulation can be exploited by pathogens and tumors to block rapid gene activation which in turn makes CMI dysfunctional. In basic terms, this is how pathogens make human cells vulnerable to attack.

Some of the environmental toxins which impair chromatin inducibility by causing harmful epigenetic reprogramming are synthetic food dyes and Roundup (glyphosate). Chronic oxidative stress & chronic inflammation also induce harmful epigenetic reprogramming which causes gene silencing which in turn causes dysfunctional CMI. The prevalence of population-scale chronic oxidative stress and chronic inflammation is far more extensive than people realize because these problems exist in silence. They can't be felt because there are no sensory nerves enervating vascular endothelial cells or tissues in the brain. Chronic oxidative stress & chronic inflammation are caused by the Western diet, by microplastics deposited in tissues throughout the body including the brain, by dormant reservoirs of live Covid virus and by persistent spike protein which may circulate in the body indefinitely just to name a few. These problems also induce chronic endothelial dysfunction, chronic inflammation, chronic mitochondrial impairment, chronic impairment of DNA repair and the down regulation of tumor suppressor genes.

Bidirectional relationships exist between chronic oxidative stress, chronic inflammation, chronic endothelial dysfunction and chronic mitochondrial impairment and harmful epigenetic reprogramming. Vicious cycles exist causing each chronic pathology to exacerbate harmful epigenetic reprogramming and vice versa. For example, endothelial dysfunction and chronic inflammation exacerbate harmful epigenetic reprogramming, and harmful epigenetic reprogramming exacerbates chronic endothelial dysfunction and chronic inflammation. The underlying cause of these dangerous feedback loops is dysfunctional CMI. The restoration and maintenance of functional CMI is the only way to break the vicious cycles which can't be felt or seen while they are taking place. Population-scale restoration and maintenance of functional CMI is tantamount to the survival of modern nations in the new age of epigenetic warfare.

Only a few percent of Americans have fully functioning immunity against bioweapons. The first line immune defenses against bioweapons come from a part of the human immune system that few people know we have and few know we need. It's called cell-mediated immunity (CMI). It is comprised of the innate branch of the immune system plus a part of the adaptive branch of the immune system in the form of T cells and dendritic cells. CMI does not include the B cell component of the adaptive branch of the immune system which manufacture antibodies.

Disclosed is a method to mitigate an unrecognized national security threat in the form dysfunctional CMI which presently is believed to affect a substantial majority of the American population estimated at 98% including healthy people who consume a healthy diet.

Only fully functional CMI can prevent catastrophic dysregulation of CMI by pathogens. Nearly all bioweapons kill by causing lethal dysregulation, subversion and evasion of CMI, usually manifesting in the form of cytokine storm.

Covid, The Plague, Tularemia, Ebola, Marburg Virus, Hanta Virus, Monkeypox Virus, Mideast Respiratory Virus (MERS), Brucellosis, Typhus, Salmonella, Melioidosis, Ricin, Staphylococcal Enterotoxin B and Severe Influenza all kill by inducing CMI dysregulation that triggers cytokine storm.

Anthrax, Smallpox, Lassa Fever, Nipah Virus, and Shigella kill primarily by dysregulation, subversion and evasion of CMI without causing cytokine storm.

Once catastrophic dysregulation, subversion, and evasion of CMI becomes clinically apparent, it's often too late to prevent death or severely disabling injuries to organs throughout the body. In this new age of epigenetic warfare, population-scale dysregulation of CMI can cause the mass destruction of the world's most advanced militaries and the collapse of modern societies. Thus, the first priority of biodefense is to prevent catastrophic dysregulation of CMI.

The only defense against epigenetic warfare is functional CMI. Functional CMI at population-scale is the only biological defense mechanism capable of: 1) preventing mass death and the mass destruction of militaries and cities caused by population-scale immune dysregulation and cytokine storm; 2) shielding militaries and cities against future pandemics; 3) mitigating the ongoing epigenetic injuries now silently underway in the bodies of millions caused by Covid-induced harmful epigenetic and metabolic reprogramming, dormant reservoirs of live Covid virus, persistent Covid spike protein, Covid spike protein induced immune cell depletion and exhaustion, chronic dysbiosis, impairment of DNA repair, autophagy, apoptosis, trained innate immunity, unfolded protein response, exacerbation of chronic oxidative stress, chronic inflammation, chronic immunomodulatory dysfunction, endoplasmic reticulum stress, endothelial dysfunction, mitochondrial impairment and the induction of chronic renin-angiotensin dysregulation with associated fibrosis in vital organs.

Immunological Fitness (TM) is a turnkey solution to restore and maintain functional CMI for individuals and populations. The method of Immunological Fitness offers the only means to protect the US military and American cities against mass death caused by immune dysregulation and cytokine storm because it represents the only way to transform dysfunctional CMI to functional CMI.

CMI is the first line of immune defense against bioweapons. Immunological fitness works by transforming dysfunctional CMI to functional CMI. The foundation of CMI functionality rests upon rapid activation of hundreds of genes at the moment the immune system detects a pathogen attempting to invade the body. Rapid gene activation requires the ability to convert closed chromatin, known as heterochromatin to open chromatin, known as euchromatin. The complex molecular processes which facilitate the conversion of heterochromatin to euchromatin are collectively called making chromatin inducible. For chromatin inducibility to take place, only specialized transcription factors called pioneer transcription factors are capable of initiating the process of transforming heterochromatin to euchromatin. However, for pioneer transcription factors to accomplish this task, vitamin D receptors (VDR) saturated with the active metabolite of vitamin D (1,25[OH]2-vitamin D) must be present. No other molecule, whether non-pharmaceutical or pharmaceutical, can saturate the VDR and activate pioneer transcription factors except for the active metabolite of vitamin D. VDR saturation is a critical rate-limiting factor which ultimately controls the success or failure of chromatin inducibility.

The failure of chromatin inducibility is the root cause of dysfunctional CMI and immune dysfunction overall. Restoring chromatin inducibility is an essential first step required to prevent immune dysregulation, cytokine storm, and to defend the body against invading pathogens. Impairment of the process of converting heterochromatin to euchromatin is a root cause of dysfunctional CMI because it impedes a critical first step required for the immune system to defend the body against invading pathogens.

Chromatin inducibility is controlled by the epigenome. Corruption of the epigenome by harmful epigenetic reprogramming down regulates the process of chromatin inducibility by pathologically altering the three-dimensional configuration of the epigenome. By interfering with chromatin inducibility, pathogens cause potentially permanent population-scale immune dysfunction.

About 90% of a cell's chromatin is closed at any given time. Defective chromatin inducibility caused by unsaturated and/or dysfunctional vitamin D has profound downstream impacts on the cell's ability to transcribe DNA and synthesize proteins and other life critical molecules.

By impeding chromatin inducibility, harmful epigenetic reprogramming induced by pathogens, cancers, and environmental toxins induces gene silencing. Gene silencing is a double-edged sword: while it is a vital component of innate immune defense and tolerance, its dysregulation can be exploited by pathogens and tumors which block rapid gene activation which in turn makes CMI dysfunctional. Gene silencing is key to understanding how harmful epigenetic reprogramming makes human cells vulnerable to pathogens, cancers and a wide range of chronic diseases.

Chronic oxidative stress and chronic inflammation also induce harmful epigenetic reprogramming which in turn causes gene silencing. Environmental toxins such as certain synthetic food dyes as well as glyphosate (Roundup) do the same.

The prevalence of injuries caused by population-scale chronic oxidative stress and chronic inflammation is far more extensive than people realize because they can't feel these injuries while the injuries are taking place. The tissues under attack lack sensory nerves and remain numb to pain while the injuries are taking place. Presently, tens of millions of Americans are experiencing continuous injuries to their brains, endothelium, nervous systems, gut microbiomes and many internal organs yet they are entirely unaware that their bodies are under attack.

Chronic oxidative stress and chronic inflammation caused by: (1) the Western diet; (2) microplastics deposited in tissues throughout the body including the brain; (3) dormant reservoirs of live Covid virus and (4) persistent spike protein which may circulate in the body indefinitely each induce gene silencing which in turn induces chronic endothelial dysfunction, chronic mitochondrial impairment, chronic impairment of DNA repair and the down regulation of tumor suppressor genes.

Bidirectional relationships exist between harmful epigenetic reprogramming and chronic oxidative stress, chronic inflammation, chronic endothelial dysfunction and chronic mitochondrial impairment. These bidirectional relationships induce vicious cycles which cause these chronic pathologies to exacerbate harmful epigenetic reprogramming and vice versa. For example, endothelial dysfunction and chronic inflammation both exacerbate harmful epigenetic reprograming and harmful epigenetic reprogramming exacerbates both chronic endothelial dysfunction and chronic inflammation. However, another bidirectional relationship forms the foundation of the entire human immune system. It is comprised of the two-way feedback loop between vitamin D saturation and intracellular glutathione. Saturated VDRs regulate the production of glutathione and glutathione independently regulates the functionality of vitamin D. For example, the serum vitamin D level could be 90 ng/mL (reflecting full saturation of the VDR and a steady state of zero-order pharmacokinetics). However, if intracellular levels of glutathione are low, vitamin D functionality is negatively affected. There is a strong association between low glutathione and the widely unknown problem of low responders to vitamin D. The scientific literature reports that up to 40% of people are low responders to vitamin D.

The same epigenetic and metabolic vicious cycles that make entire populations vulnerable to bioweapons also cause population-scale vulnerability to autism, Alzheimer's, atherosclerotic heart disease, type II diabetes, obesity, cancer, and more.

What fuels these vicious cycles is impaired immunological fitness. The only way to stop these vicious cycles is by normalizing immunological fitness. In this new age of epigenetic warfare, population-scale normalization of immunological fitness is tantamount to the survival of nations.

The epidemic of chronic diseases plaguing the United States cannot be stopped merely by eating healthy, living healthy and new regulations which mandate the removal of toxic compounds in foods and the environment. The only way to stop the chronic disease epidemic is to stop the vicious cycles which perpetuate harmful epigenetic reprogramming.

Defending the US military and the American people against epigenetic weapons will simultaneously reverse the chronic disease epidemic at no additional cost. By upgrading national biodefense to survive epigenetic warfare, America's chronic disease epidemic will simultaneously come to an end.

Whether induced by pathogens, cancer, metabolic derangements or toxic exposures coming from food or the environment, harmful epigenetic reprogramming ravages human cells by unleashing a nexus of self-amplifying molecular attacks which attempt to destabilize the foundations of cellular self-defense such as: (1) the inducibility of chromatin; (2) the biological rheostats which provide: (a) continuous buffering against oxidative stress: (b) immunomodulation which balance pro-inflammatory and anti-inflammatory CD4+ effector T cells; (c) anti-inflammation which regulates cell signaling that controls the production and release of cytokines and (d) regulation of mitochondrial energy production through NAD+ and sirtuins; (3) the metabolic switching which activates or deactivates natural killer (NK) cell functionality; (4) the beneficial bacteria in the gut which produce the epigenetic regulator butyrate; (5) the regulation of cellular repair through autophagy; (6) the regulation of cell cycle progression through apoptosis and the production of first line molecular defenses against attack such as host defense peptides, heat shock proteins and virulence factors.

The success or failure of molecular attacks by pathogens is largely dependent upon the presence or absence of easily reversible cellular vulnerabilities which leave the foundations of cellular defense wide open to devastating molecular attacks by bioweapons. These cellular vulnerabilities have been well-characterized in the scientific literature for years. Despite the fact that each of these cellular vulnerabilities is well known, nothing has been done to reverse these cellular vulnerabilities that make devastating bioweapons attacks on the US military and American cities possible. Until these cellular vulnerabilities are remediated on a population scale, the risk of mass death and societal collapse will remain.

US military leadership may be unaware of the existential perils caused by unchecked cellular vulnerabilities to molecular warfare. It is ironic that population-scale protections against these potentially devastating cellular vulnerabilities are comprised of widely available, inexpensive, non-pharmaceutical nutritional compounds which have well-established safety profiles.

While each of these cellular vulnerabilities are dangerous, as a group they are ominous because each individual vulnerability reinforces the destructive potential of the group. These self-amplifying reinforcements explain why the cascade of immune dysregulation which leads to cytokine storm is very difficult to stop once it begins. The fact that none of these cellular vulnerabilities are being addressed at population-scale explains the approximately 98% estimate of Americans is believed to have dysfunctional CMI which places them at increased risk of death or permanent disability from epigenetic weapon-induced immune dysregulation and cytokine storm.

In summary, blind spots in national biodefense preparedness have left the US military and American cities wide open to attack by devastating epigenetic weapons because these cellular vulnerabilities to molecular warfare have not been reversed at population scale. It doesn't have to be this way.

The method of immunological fitness disclosed herein systematically reverses cellular vulnerabilities to molecular warfare.

The blind spots to national biodefense preparedness are comprised of: (1) zero-order pharmacokinetics of vitamin D; (2) widespread polymorphisms affecting both the TCA cycle and the VDR; (3) population-scale deficiencies of critical micronutrients; (4) population-scale deficiencies of conditionally essential nutrients; (5) population-scale dysbiosis; (6) conditional deficiencies of dietary non-coding microRNAs; (7) population-scale impairment of essential master regulators which increase vulnerability to immune dysregulation and cytokine storm; and (8) population-scale vulnerability to cellular hijacking by pathogens. Safety will only come when all of these blind spots are corrected simultaneously at population-scale.

Zero-order pharmacokinetics of vitamin D refers to a continuous homeostasis where vitamin D receptors are always saturated. Vitamin D is a fat soluble secosteroid hormone. To achieve zero-order pharmacokinetics, levels of vitamin D stored in a person's body fat must be adequate to maintain a steady state of vitamin D receptor (VDR) saturation by the ligand 1,25(OH)₂D₃ vitamin D. This condition generally occurs when the serum level of the biologically active metabolite of vitamin D, also known as calcitriol or 1,25(OH)₂D₃ vitamin D, is above 50 ng/mL. However, because of polymorphisms which may be present in the VDR, the binding of the vitamin D ligand is variable from person to person. For this reason, the only way to assess the likelihood that a person has achieved zero-order pharmacokinetics is by blood testing to determine the serum vitamin D level. In summary, because of person-to-person variability regarding the fat reserves of vitamin D and variability related VDR polymorphisms, the disclosed method strives to achieve serum vitamin D levels in the upper end of the normal range between 65 and 100 ng/mL.

An embodiment of Immunological Fitness provides a comprehensive solution to restore and maintain optimal levels of intracellular glutathione which is key to mitigating the problem of low responders to vitamin D.

While oral glutathione supplementation may be marginally helpful to elevate low serum glutathione, it does nothing to resolve the five principle underlying causes of low intracellular glutathione.

An embodiment of Immunological Fitness rapidly and simultaneously overcomes the five principal causes of low intracellular glutathione: (1) Deficiencies of vitamin B12, B9, B6, and B2 caused by polymorphisms and/or inflammation which impair glutathione production; (2) critical micronutrient deficiencies; (3) conditional essential nutrient deficiencies; (4) microRNA deficiencies and (5) gut microbiome dysbiosis.

The most common polymorphism causing low intracellular glutathione is the MTHFR 677C>T polymorphism, a common genetic variant in the methylenetetrahydrofolate reductase (MTHFR) gene. It impairs the enzymatic conversion of vitamin B9 (folate) to its active metabolite 5-methyltetrahydrofolate (5-MTHF). According to the 1000 Genomes Project, approximately 25% of the global population are carriers of MTHFR 677C>T, Hispanics being the population with the highest frequency (47%), followed by Europeans (36%), East Asians (30%), South Asians (12%) and Africans (9%). The severity of the impact on intracellular glutathione is determined by whether a carrier of the MTHFR 677C>T gene is homozygous or heterozygous. Deficiencies of 5-MTHF also result in elevated levels of homocysteine which is associated with significantly increased risks of atherosclerosis, heart disease, strokes and cancer. Hyperhomocystinemia is associated with disease severity in Covid. It is proinflammatory and contributes to the dysregulation of CMI leading to cytokine storm. It also induces blood clots and fibrosis.

Polymorphisms affecting vitamin B12 may decrease the production of B12 which may further contribute to low intracellular glutathione and increased oxidative stress during sepsis. Though vitamin B12 deficiency does not cause a direct, depletion of glutathione, it disrupts the metabolic pathways necessary for glutathione synthesis, leading to a functional intracellular deficiency and contributing to oxidative stress.

Levels of vitamin B6 decline in the presence of inflammation. The decline can be significant in sepsis. Vitamin B6 deficiencies contribute to elevated homocysteine and may also contribute to low glutathione. Low levels of pyridoxal 5′-phosphate (PLP), the active form of vitamin B6, can inhibit lymphocyte migration and lead to lymphopenia and local exacerbation of inflammatory processes, increasing the secretion of pro-inflammatory cytokines. This dysregulation may contribute to uncontrolled immune activation, such as that seen in a cytokine storm.

Subclinical riboflavin (vitamin B2) deficiency can interfere with folate metabolism, particularly in individuals with the MTHFR C677T polymorphism, potentially exacerbating functional folate deficiency. Subclinical deficiencies of vitamin B2, where individuals have low riboflavin status without exhibiting obvious clinical signs, are likely widespread in the United States population, although they often go undetected because riboflavin biomarkers are rarely measured in human studies. This suggests that riboflavin deficiency can impair the one-carbon metabolism pathway, leading to a functional folate deficiency even in the absence of overt clinical signs. Riboflavin, in its cofactor forms flavin adenine dinucleotide (FAD) and flavin mononucleotide (FMN), plays fundamental roles in energy metabolism, cellular antioxidant potential, and metabolic interactions with other micronutrients, including iron, vitamin B6, and folate.

Critical micronutrient deficiencies may directly or indirectly contribute to the problem of vitamin D low responders as well as to dysregulation of CMI and the induction of cytokine storm.

Vitamin A: Retinoic acid, the active metabolite of vitamin A (retinol), plays a crucial role in immune function and has been implicated in the pathogenesis of COVID-19 and the induction of cytokine storm. Research suggests that the body's retinoid signaling, which depends on retinol and retinoic acid, becomes impaired during severe SARS-CoV-2 infection due to rapid depletion of these compounds. This depletion is thought to be driven by the virus's large 30 kB RNA genome, which triggers an overwhelming immune response involving the RIG-I pathway—a process that consumes significant amounts of retinoic acid for Type I interferon (IFN) synthesis. When retinoic acid stores are exhausted, Type I IFN production halts, leading to a collapse of the innate immune response.

Vitamin C: Vitamin C deficiency can contribute to low glutathione levels. Research indicates a strong functional interdependence between the two antioxidants. When glutathione levels are depleted, tissue levels of ascorbic acid (vitamin C) also decrease, suggesting that glutathione plays a role in the metabolism or maintenance of vitamin C. Conversely, studies show that vitamin C supplementation can increase glutathione levels in various tissues and cells. For instance, vitamin C supplementation in individuals with vitamin C deficiency led to an 18% increase in lymphocyte glutathione levels.

Vitamin E: Vitamin E deficiency does not directly cause low glutathione levels, but it plays a crucial role in protecting cells from oxidative stress that can deplete glutathione. Vitamin E is a fat-soluble antioxidant that helps prevent lipid peroxidation and hemolysis by neutralizing reactive oxygen species generated from intracellular hydrogen peroxide. In the absence of vitamin E, red blood cells are more susceptible to oxidative damage, leading to increased hemolysis and methemoglobin formation, which are linked to oxidative stress.

Vitamin K2: Vitamin K2 deficiency has been associated with cytokine storm, thrombotic complications, and severe disease outcomes in COVID-19 patients, suggesting a key role for vitamin K in modulating the pathological response to infection.

Selenium: Selenium deficiency is associated with reduced activity of glutathione peroxidases, which are selenium-dependent enzymes critical for antioxidant defense. This deficiency can impair the body's ability to manage oxidative stress, potentially leading to lower levels of reduced glutathione (GSH) and increased oxidative damage. In critically ill patients, especially those with sepsis, selenium deficiency is common and correlates with disease severity. Selenium deficiency is associated with elevated levels of key pro-inflammatory cytokines involved in cytokine storms, including IL-6, IL-1β, and TNF-α.

Magnesium: Magnesium deficiency can contribute to a poor response to vitamin D supplementation and is known to contribute to the problem of low vitamin D responders. Magnesium is a critical cofactor in several steps of vitamin D metabolism, including the activation of vitamin D into its active form, 1,25-dihydroxyvitamin D, and the synthesis of vitamin D binding protein (VDBP). Clinical studies have shown that magnesium supplementation can reverse resistance to vitamin D treatment in deficient patients.

Zinc: Zinc deficiency can contribute to a reduced response to vitamin D, as zinc is an essential cofactor for the vitamin D receptor (VDR) to function properly. The VDR requires zinc to maintain its correct structural conformation, which is necessary for it to bind to DNA and regulate the expression of vitamin D-dependent genes. Inadequate serum zinc levels may underlie the variability in individual responses to vitamin D supplementation, suggesting that zinc deficiency can contribute to poor vitamin D responsiveness. Zinc deficiency has also been linked to a poor prognosis due to altered cytokine release and impaired immune regulation, potentially facilitating the uncontrolled inflammation characteristically associated with cytokine storm.

Copper: Copper deficiency is associated with impaired immune function, which can increase susceptibility to infections and potentially contribute to dysregulated immune responses. Copper deficiency impairs both humoral and cell-mediated immunity, reduces the proliferation of T cells, decreases interleukin-2 (IL-2) secretion, and diminishes the function of neutrophils and macrophages.

Boron: Boron deficiency may contribute to the problem of low responders to vitamin D supplementation. Research suggests that boron can up-regulate serum 25-hydroxyvitamin D levels, potentially by inhibiting the enzyme 24-hydroxylase, which is responsible for the catabolism of vitamin D. This inhibition could enhance the effectiveness of vitamin D, meaning individuals with low boron levels might have a diminished response to vitamin D intake. Studies have shown that boron supplementation can increase serum 25(OH)D3 levels significantly, with one pilot study reporting an average rise of 20%. Research also shows that boron supplementation can inhibit the NF-κB pathway, a key regulator of pro-inflammatory cytokine genes, thereby reducing the expression of TNF-α, IL-1β, MIP-1α, and iNOS in macrophages. This suggests that adequate boron levels are necessary to maintain immune balance and prevent excessive cytokine production. Boron deficiency may impair immune regulatory function, potentially increasing susceptibility to cytokine storm.

Omega-3 fatty acids: Omega-3 fatty acids, including eicosapentaenoic acid (EPA) and docosahexaenoic acid (DHA), exert anti-inflammatory effects by inhibiting the formation of pro-inflammatory eicosanoids derived from arachidonic acid (an omega-6 fatty acid) and by forming anti-inflammatory lipid mediators such as resolvins and protectins. These mechanisms suppress the activity of nuclear transcription factors like NF-κB, which are key drivers of pro-inflammatory cytokine production. Maintaining a low omega-6/omega-3 ratio through adequate omega-3 intake is considered a crucial strategy for reducing the risk of inflammatory cytokine storms.

R-Alpha Lipoic Acid: Alpha-lipoic acid (ALA) may help lower the risk of cytokine storm by modulating inflammation and oxidative stress, both of which are key factors in the development of this condition. ALA has been shown to inhibit the activation of NF-κB, a transcription factor that regulates the expression of pro-inflammatory cytokines such as IL-1β and IL-6, which are central to cytokine storm dynamics.

Conditionally essential amino acids are those that are normally nonessential but become essential under specific physiological conditions such as illness, injury, or stress, when the body's demand exceeds its ability to synthesize or metabolize them.

Glutathione (GSH) is composed of the amino acids cysteine, glycine, and glutamate. Its synthesis is dependent on the availability of these three amino acids, with cysteine being the rate-limiting precursor. When the body experiences stress, illness, or injury, the demand for glutathione increases, and the endogenous production of its precursors, particularly cysteine and glycine, may be insufficient, making them conditionally essential.

Deficiencies of five conditionally essential amino acids are associated with low glutathione. These are arginine, cysteine, glutamine, glycine, and serine. Beyond their impacts on glutathione, these amino acids play critical roles in various bodily functions, including protein synthesis, immune function, detoxification, and antioxidant defense.

Glutathione (GSH) consists of cysteine, glycine, and glutamate. Glutamine is the first and rate-limiting step in glutathione biosynthesis and is crucial for providing the necessary substrate for glutathione production.

Because glycine and serine are closely linked metabolically through the enzyme serine hydroxymethyltransferase (SHMT), deficiencies in one often coincide with the other, and co-administration of glycine and serine may be more effective than glycine alone in deficient states.

Arginine is a precursor for glutamine. It is metabolized to produce nitric oxide (NO). Studies have shown that l-arginine supplementation can induce glutathione synthesis in tissues like brown adipose tissue by activating the expression of glutamate-cysteine ligase (GCL), the rate-limiting enzyme in glutathione production. This process is mediated through nitric oxide signaling pathways. Therefore, a deficiency in arginine can impair this pathway, potentially leading to reduced glutathione synthesis and lower overall glutathione levels, especially under conditions of stress or disease.

MicroRNAs can impact low GSH levels by regulating the expression of genes involved in GSH synthesis and metabolism. Dysregulation of specific miRNAs has been linked to decreased GSH levels, contributing to oxidative stress, which is implicated in various diseases including viral infections.

Gut microbiome dysbiosis can impact low glutathione levels. Dysbiosis, an imbalance in the gut microbiota, is associated with increased production of reactive oxygen species (ROS), which can deplete glutathione. Research indicates that dysbiosis contributes to oxidative stress, which in turn can impair glutathione synthesis and function.

Immunological Fitness creates the foundation for continuous cellular defense against biological attacks by restoring and maintaining: (1) a continuous state of chromatin inducibility which counteracts pathologic gene silencing; (2) biological buffers which serve as rheostats that continuously protect cells against damaging oxidative stress in the form of harmful levels of intracellular and extracellular reactive oxygen species (ROS) and reactive nitrogen species (RNS); (3) biological rheostats that regulate the production of cellular energy; (4) metabolic switching of critical epigenetic processes which control the formation of harmful epigenetic memory as well as the function of innate immune cells such as natural killer cells; (5) metabolic signaling networks which modulate a diverse array of cellular functions including the production and release of pro-inflammatory and anti-inflammatory cytokines which prevent immune dysregulation and cytokine storm; (6) a healthy gut microbiome; (7) tight junctions which lead to leaky gut syndrome, leaky vascular endothelium and impaired integrity of the blood-brain barrier; (8) the glycocalyx found on the luminal surfaces of vascular endothelium throughout the body including in the blood-brain barrier and pulmonary vasculature as well as on the gut epithelium. The glycocalyx regulates vascular permeability and acts as a barrier to prevent the spread of inflammation; (9) tumor suppressor genes such as p53 and BRCA genes which also carry out DNA repair; (10) autophagy and mitophagy which protect cellular and mitochondrial health and (11) regulatory control of endoplasmic reticulum stress and the unfolded protein response.

While Immunological Fitness is the foundation of continuous cellular defenses against biological attacks, the continuous homeostasis of vitamin D saturation and functionality is the foundation of Immunological Fitness.

The continuous homeostasis of vitamin D saturation and functionality is also the foundation of numerous master regulators of specific cellular functions because continuously optimized vitamin D saturation and functionality impacts whether each of these master regulators of specific cell functions are fully functional. The master regulators are:

Nuclear factor erythroid 2-related factor 2 (NRF2): NRF2 is a master regulator of tissue damage control and disease tolerance to infections. It is a transcription factor that regulates the cellular defense against toxic and oxidative insults through the expression of genes involved in oxidative stress response and drug detoxification. NRF2 activation renders cells resistant to chemical carcinogens and inflammatory challenges. Vitamin D plays a significant role in enhancing the function of the NRF2 signaling pathway, which is a critical regulator of cellular antioxidant defenses. The active form of vitamin D, 1,25-dihydroxyvitamin D, exerts an antioxidant effect by transcriptionally upregulating NRF2 through the VDR.

Peroxisome proliferator-activated receptors (PPARs): PPAR's are master regulators of energy metabolism and play a significant role in modulating the host response to Covid infection, primarily through their effects on inflammation, lipid metabolism, and immune regulation. Covid infection has been shown to downregulate PPAR expression in tissues such as lung epithelial cells, peripheral blood mononuclear cells (PBMCs), and bronchoalveolar lavage fluids, which correlates with increased proinflammatory cytokine production, including IL-6, IL-1β, and TNF-α, and the development of cytokine storm—a hallmark of severe Covid. Vitamin D plays a significant role in regulating PPARs which are crucial for metabolic homeostasis.

Activated protein kinase (AMPK): AMPK plays a significant role in the cellular response to Covid. It functions as a key regulator of cellular energy balance, influencing processes such as autophagy, lipid metabolism, and immune responses, all of which are relevant to viral infection. Covid infection has been shown to inhibit AMPK activity, contributing to metabolic dysregulation, including disruptions in lipid metabolism such as increased LDL and triglycerides and decreased HDL, resembling a hyperlipidemic state. The inhibition of AMPK by Covid supports viral replication, as the virus exploits host lipid pathways and hijacks autophagy to create viral factories necessary for its propagation. Conversely, pharmacological activation of AMPK has demonstrated antiviral effects against Covid. Vitamin D plays a significant role in the function of AMPK. Research indicates that vitamin D supplementation can enhance AMPK activation. In studies using C2C 12 skeletal muscle cells, treatment with 1,25-dihydroxyvitamin D (1,25(OH)2D) led to a significant increase in the activation of AMPK, along with increased SIRT1 activity, which is closely associated with AMPK function.

Class IA phosphatidylinositol 3-kinase/Protein kinase B/Mammalian target of rapamycin (PI3/AKT/mTOR): The PI3/AKT/mTOR signaling pathway is widely recognized as a master regulator of cellular processes. It functions as a central hub that integrates signals from nutrients, growth factors, energy status, and stress to control fundamental cellular activities such as growth, metabolism, proliferation, survival, and protein synthesis. It acts as a master switch for energetically demanding processes, promoting anabolism when nutrients are sufficient and allowing autophagy when nutrients are limited. Vitamin D plays a significant role in the function of the PI3K/AKT/mTOR signaling pathway, acting through multiple mechanisms. Vitamin D enhances insulin signaling in neurons by increasing the phosphorylation of Akt in a PI3K-dependent manner, which is crucial for downstream metabolic and cellular functions. This effect is mediated through the VDR and involves the regulation of key genes within the PI3K pathway.

Inositol Phosphate Signaling: The inositol phosphate signaling pathway plays a central and highly coordinated role in cellular function, influencing a vast array of processes from metabolism and energy homeostasis to calcium signaling and neuronal function, consistent with its role as a master regulator of cellular information flow. It is also deeply involved in energy processing decisions, regulating growth factor signaling, nutrient sensing, and the maintenance of cellular energy stores through its interactions with key systems like AKT, AMPK, and mTOR. Vitamin D plays a significant role in regulating the inositol phosphate signaling pathway, primarily through its rapid, non-genomic actions mediated by a membrane-associated vitamin D receptor. Upon binding of 1α,25(OH)₂D₃ to mVDR, signaling pathways such as protein kinase C (PKC) and adenylate cyclase (AC) are activated. This activation leads to the elevation of polyisoprenyl phosphate (PIPP) levels, which triggers the formation of inositol triphosphate (IP₃). The rise in IP3 levels is associated with a rapid increase in intracellular calcium ([Ca²⁺]ᵢ), which occurs within 2-5 minutes of receptor activation.

Zonulin: Zonulin is called the master regulator of tight junctions. Zonulin is a protein that regulates the permeability of the intestinal epithelial barrier, the blood-brain barrier (BBB) and blood vessels by modulating tight junctions. When zonulin levels are appropriately regulated, it helps maintain the integrity of these junctions. However, dysregulated zonulin production can lead to increased permeability in the gut, brain and blood vessels. In the gut this is known as “leaky gut” syndrome characterized by the leaking of large molecules such as undigested food proteins, toxins, and bacteria which enter the bloodstream, potentially triggering systemic inflammation that may contribute to the induction of cytokine storm. Vitamin D appears to play a significant role in regulating zonulin. Research indicates that vitamin D deficiency leads to increased zonulin expression and elevated serum zonulin levels, which are associated with impaired tight junctions. Conversely, vitamin D supplementation or the active metabolite calcitriol (VD3) has been shown to suppress zonulin release and inhibit the signaling pathway that induces its production, thereby helping to maintain gut barrier integrity and tight junctions throughout the body.

Interleukin-1 receptor associated kinase (IRAK)-M: IRAK-M acts as a negative regulator of Toll-like receptor (TLR) signaling and controls the magnitude of inflammatory responses in macrophages. In the context of Covid infection, the virus's spike protein interacts with the angiotensin-converting enzyme 2 receptor on macrophages, leading to the suppression of IRAK-M expression at both the mRNA and protein levels. This downregulation of IRAK-M renders macrophages hyper-responsive to TLR ligands, such as LPS and PAM3csk4, resulting in increased production of pro-inflammatory cytokines including IL-6, TNFα, and MIP1a. This mechanism contributes to excessive inflammatory response and cytokine storm, which is a major cause of mortality in Covid patients.

Mitogen-Activated Protein Kinase/Extracellular Signal-Regulated Kinase (MAPK/ERK) Signaling Pathway: The MAPK/ERK signaling pathway plays a central role in the induction of cytokine storm. Activation of the MAPK pathway, particularly the ERK1/2 branch, is a key mechanism in this process. In severe cases of diseases like Covid, infection leads to the activation of MAPK signaling through the disruption of ACE2, the viral receptor, which normally acts as a negative regulator of MAPK signaling. This disruption results in the activation of transcription factors such as NF-κB and AP-1, which drive the production of proinflammatory cytokines including IL-6, TNF-α, IL-17, and IFN-γ. The uncontrolled activation of macrophages, a hallmark of severe Covid, is linked to ERK1/2 activation, which also inhibits type-I interferon production, further disrupting immune coordination. In human adipocytes, vitamin D acts as a potent negative regulator of the MAPK signaling pathway, specifically inhibiting the activation of p38 MAPK and Erk1/2 kinases. This inhibition occurs in a dose-dependent manner and contributes to the reduction of proinflammatory chemokine release. The mechanism involves the downregulation of phosphorylated p38 MAPK and Erk1/2, which are key components of the MAPK pathway.

Hypoxia-Inducible Factor-1 Alpha (HIF-1α): HIF-1α is a master transcriptional regulator of the adaptive response to hypoxia. HIF-1α plays a critical role in the pathogenesis of cytokine storms in severe viral respiratory infections. HIF-1α is activated by viral proteins like ORF3a, which induces mitochondrial damage and ROS production, leading to increased HIF-1α expression. This upregulation of HIF-1α promotes Covid infection and exacerbates inflammatory responses, contributing to the cytokine storm. Vitamin D plays a significant role in the function of HIF-1α, although the mechanism appears to be context-dependent. Vitamin D effects are mediated via the VDR and potentially involves vitamin D response elements (VDRE) in the HIF-1α promoter.

Sterol regulatory element-binding protein (SREBP): SREBP is a master regulator of lipid homeostasis. Covid activates the SREBP transcription factors, SREBP1 and SREBP2, which play crucial roles in the virus's replication and pathogenesis. The virus reprograms host cell lipid metabolism by increasing the activation of these transcription factors, leading to elevated levels of enzymes like DGAT1 and PLIN2, and promoting cholesterol and triglyceride synthesis. This results in lipid droplet (LD) maturation and biogenesis, processes that support Covid replication. Vitamin D acts as an inhibitor of SREBP activation by inducing the proteolytic processing and ubiquitin-mediated degradation of SREBP cleavage-activating protein, an essential escort protein for SREBP. This process occurs independently of the VDR.

Tumor necrosis factor-alpha (TNF-α): TNF-α is widely recognized as a master regulator of the immune response and inflammation. It is considered a key initiator of immune-mediated inflammation in multiple organ systems, including the brain. TNF-α is one of the first signals released by macrophages and other white blood cells in response to injury or infection, rapidly triggering the inflammatory process. It plays a pivotal role in orchestrating the production of a pro-inflammatory cytokine cascade and is a central player in the development of many chronic inflammatory and autoimmune diseases. Its role as a master regulator is further underscored by its ability to induce the expression of other cytokines, chemokines, and inflammatory mediators. TNFα is a key pro-inflammatory cytokine that contributes to the cytokine storm observed in severe cases of Covid. It synergizes with interferon-gamma to drive inflammatory cell death, tissue damage, and mortality in Covid infections and cytokine shock syndromes. Vitamin D down-regulates TNF-α, particularly in immune cells like macrophages. Studies show that both 25(OH)D and its active form, 1.25(OH)₂D₃, significantly inhibit TNF-α expression in fully differentiated human monocyte-derived macrophages, both under normal conditions and during pro-inflammatory stimulation with LPS. This effect is linked to a reduction in NF-κB expression, a key transcription factor for TNF-α, and is supported by findings that vitamin D induces a shift from a pro-inflammatory “M1” macrophage phenotype to an anti-inflammatory “M2” phenotype.

Transforming growth factor beta (TGF-β): TGF-β is a master regulator of the interplay between the gut microbiota and host immune cells, influencing regulatory T cells, Th17 cells, innate lymphoid cells, and B cells. It is also characterized as a master regulator of fibrosis. TGF-β plays a significant and complex role in cytokine storm, particularly in the context of severe infections like Covid. Research suggests that an abnormally increased levels of TGF-β is a basic feature of the cytokine storm associated with Covid infection, leading to a disequilibrated cytokine network. This dysregulation is linked to key clinical manifestations of Covid, including fatigue, low-grade fever, dry cough, interstitial lung changes, and the loss of olfactory and gustatory senses. TGF-β contributes to these symptoms by promoting fibrosis, increasing mucus secretion, suppressing immune function and inhibiting lymphocyte proliferation. The clinical severity of Covid, ranging from asymptomatic to critical, correlates with the level of this cytokine storm, with TGF-β being the dominant factor in cases characterized by fatigue and mild fever. Vitamin D acts as a negative regulator of TGF-β signaling through a complex, reciprocal relationship. TGF-β can induce the expression of the VDR, and the active form of vitamin D, 1,25(OH)₂D₃, then exerts a negative feedback on TGF-β activity. This inhibition is VDR-dependent and includes the suppression of TGF-β-induced mitochondrial metabolic changes, such as increased mitochondrial membrane potential, ATP production, and ROS.

Janus kinase/signal transducer and activator of transcription (JAK/STAT) signaling: The JAK-STAT signaling pathway plays a crucial role in the immune response to Covid and is significantly involved in the pathophysiology of severe disease. This pathway transmits signals from cytokines and growth factors to the cell nucleus, regulating gene expression related to immune regulation, inflammation, and hematopoiesis. The dysregulated immune response which leads to cytokine storm is characterized by elevated levels of pro-inflammatory cytokines such as IL-2, IL-6, IL-8, TNF-α, IFN-γ, and others, many of which signal through the JAK-STAT pathway. Vitamin D enhances the interferon-α induced JAK-STAT signaling pathway, which promotes antiviral defense by increasing the expression of interferon-stimulated genes and reducing viral load.

Suppressors of cytokine signaling (SOCS): SOCS proteins function as master regulators of cytokine signaling and immune regulation by combining direct inhibitory interactions with cytokine receptors and signaling proteins with a generic mechanism of targeting associated proteins for proteasomal degradation via their SOCS box domain, which recruits E3 ubiquitin ligase complexes. This dual mechanism allows SOCS proteins to effectively terminate signals from a wide array of cytokines, growth factors, and interferons. Their role extends beyond simple signal termination; they are crucial for regulating immune cell development, differentiation, and function, including the polarization of CD4+ T cells into Th1, Th2, Th17, and T regulatory lineages, as well as the maturation of CD8+ T cells. Vitamin D upregulates SOCS-1 and SOCS-3 in neutrophils thereby limiting excessive inflammatory cytokine production.

T-box expressed in T cells (T-Bet) transcription factor: The T-bet transcription factor has long been considered the master regulator of the Th1 cell lineage and type 1 immune responses due to its essential role in initiating Th1 differentiation from naive T cells and driving the expression of key cytokines like interferon-gamma (IFN-γ). It functions by promoting the expression of proinflammatory cytokines and chemokines while simultaneously suppressing the development of other T helper cell subsets, such as Th2 and Th17, through mechanisms like sequestering GATA3 and silencing key genes via epigenetic modifications. Recent research indicates its function extends beyond being a simple master regulator of Th1 cells. T-bet is now recognized as a key player in the development and function of various immune cell types, including natural killer (NK) cells, cytotoxic CD8+ T cells, innate lymphoid cells (ILCs), B cells, and even dendritic cells (DCs). The influence of vitamin D on the T-bet transcription factor involves VDR signaling. VDR signaling plays a crucial role in the development and function of specific regulatory T cell subsets, such as invariant NKT (iNKT) cells and CD8αα/TCRαβ T cells.

GATA binding protein 3 (GATA3) transcription factor: The GATA3 transcription factor is a master regulator of Th2 cell differentiation and is necessary and sufficient for the expression of Th2 cytokines, including IL-4, IL-5, and IL-13. The GATA3 transcription factor plays a significant role in regulating immune responses and cytokine production, which are central to the development of a cytokine storm. Vitamin D's regulation of gene expression, including transcription factors like GATA3, occurs through the VDR.

Notch Signaling Pathway: The Notch signaling pathway is a master regulator of cell fate decisions and plays a complex and significant role in modulating immune responses, including the regulation of cytokine production, which is central to the development of a cytokine storm. Dendritic cells utilize Notch signaling to tailor their cytokine responses to inflammatory stimuli. The Notch signaling pathway plays a significant role in the pathophysiology of Covid, influencing viral entry, inflammation, and lung regeneration. Notch signaling also promotes Covid entry by upregulating furin, a host protease that cleaves the viral spike protein, and by suppressing ADAM17, which regulates ACE2 shedding from the cell membrane. This pathway is also involved in amplifying the inflammatory response through a positive feedback loop with IL-6, where Notch activation increases IL-6 production and IL-6 in turn enhances Notch ligand expression, contributing to the cytokine storm observed in severe cases. Vitamin D regulates the Notch signaling pathway, primarily through the VDR, which acts as a transcription factor. The VDR positively regulates the Notch pathway by directly binding to the promoter region of the Notch-1 gene, thereby promoting its transcription and subsequent activation of the Notch signaling cascade. This regulatory mechanism has been demonstrated in the context of intestinal barrier protection, where VD/VDR signaling maintains tight junction integrity in colitis by up regulating Notch-1 and its downstream targets, such as Hes1.

Forkhead box P3 (FOXP3) transcription factor: The FOXP3 transcription factor functions as a master regulator in the development and function of regulatory T cells (Tregs). The FOXP3 transcription factor also play s a critical role in preventing cytokine storm by suppressing the production of key inflammatory cytokines. FOXP3 is essential for the development and function of Tregs, which maintain immune tolerance and prevent excessive immune responses. It directly represses the expression of major pro-inflammatory cytokines such as IL-2, IL-4, IFN-γ, and TNF-α. This suppression occurs through physical interactions with key transcription factors like NFAT and NF-κB, which are required for the activation of cytokine genes. By inhibiting the transcriptional activity of NFAT and NF-κB, FOXP3 prevents the uncontrolled cytokine production characteristic of a cytokine storm. Vitamin D regulates the FOXP3 transcription factor by promoting FOXP3 expression in CD4+ T cells. It directly binds to vitamin D response elements (VDREs) located within a conserved non-coding sequence (CNS) region of the human FOXP3 gene. This direct binding of the VDR to these VDREs enhances FOXP3 promoter activity in response to vitamin D. Furthermore, Treg cells are induced by 1,25(OH)2VD3 (VD-iTreg cells) suppress the proliferation of target CD4+ T cells, and this suppressive function is dependent on FOXP3 expression and cell contact. These findings indicate that 1,25(OH)2VD3 regulates human immune responses by directly modulating FOXP3 expression through VDR binding to the FOXP3 gene, which is essential for the inhibitory function of VD-iTreg cells.

In the new age of epigenetic warfare, immunological fitness is the foundation for national biodefense. Immunological Fitness represents the only way to prevent mass death and disability in the face of a bioweapon attack. Furthermore, Immunological Fitness must be functional for Continuous Cellular Defenses to be functional.

Because epigenetic warfare is asymmetric, the most advanced militaries in the world are now vulnerable and undefended against mass destruction by epigenetic weapons because the militaries lack Immunological Fitness.

Because epigenetic weapons strike without warning, militaries and societies-at-large must remain on a “war footing” against molecular warfare at all times.

A war footing requires more than Immunological Fitness. It requires: (1) continuous broad-spectrum activation of defenses against infection by pre-infection priming against bioweapon attacks. For viruses, this state of continuous priming is referred to as antiviral priming; (2) continuous defenses against hijacking of cellular immune defenses; (3) continuous defenses against new cellular injury by epigenetic weapons; (4) mitigation of existing cellular injuries and dysfunction caused by harmful epigenetic reprogramming; (5) continuous cellular defenses against new or recurrent harmful epigenetic reprogramming; (6) enhanced maintenance of master regulators beyond the baseline up-regulation provided by Immunological Fitness.

Continuous Cellular Defenses are the result of first, establishing and maintaining immunological fitness and then building upon Immunological Fitness a state of continuous cellular defenses against molecular warfare by ingesting a sophisticated yet safe, inexpensive, pleasant tasting bioavailability optimized daily ingestible comprised of bioavailability optimized non-pharmaceutical nutritional compounds. Continuous Cellular Defenses leverage healthful synergies which happen when dietary ingredients are ingested at widely accepted dosages that are in the low to mid-range of the normal range. Like eating a healthy salad comprised of a variety of vegetables and fruits, the power of the synergies between ingredients drive the protective benefits to cellular defenses. Also, many of the ingredients in these formulations have the FDA GRAS designation which means that these ingredients are “generally regarded as safe”. Many of the ingredients are plant phytochemicals which have been used for hundreds of years by traditional societies across the globe. An example is Artemisinin, a natural bioactive sesquiterpene lactone derived from the herbal medicinal plant Artemisia annua, commonly known as sweet wormwood. In 2015, the non-pharmaceutical version of Artemisinin won the Nobel prize in medicine. As an anti-malarial and antiviral compound, artemisinin has saved countless lives.

Continuous broad-spectrum activation of cellular defenses against infection begins with continuous, broad-spectrum, pre-infection priming against viruses, bacteria, fungi and parasites. To demonstrate the method, this narrative will focus on protection against viruses though many of the protections against viral infections also protect against bacteria, fungi and parasites. All the non-pharmaceutical compounds listed here are utilized in the method.

Pre-infection antiviral priming comprises: (1) viral entry blockade which occurs either by inhibition and or blocking of host cell receptors or by inhibition and or blocking of endocytosis; (2) viral replication blockade; (3) spike protein attenuation; (4) inhibition of viral induced platelet dysfunction; (5) up-regulation of host defense peptides; (6) up-regulation of pro-resolving mediators (SPMs); (7) up-regulation of cell sensing; (8) pre-activation of tissue specific immunity via innate lymphoid cells; (9) inhibition of nonstructural protein 1 (NSP1).

There are five types of receptors: (1) the receptor binding domain of the angiotensin-converting enzyme 2 (ACE2) complex; (2) transmembrane serine protease 2 (TMPRSS2) protein; (3) furin protease; (4) cluster of differentiation 147 (CD147) receptor and (5) the receptor for advanced glycation end products (RAGE)/high mobility group box 1 (HMGB1) receptor axis.

The ACE2 complex in the context of Covid refers to the structural and functional interactions involving ACE2, which serves as the primary cellular receptor for SARS-CoV-2 viral entry. The interaction between the Covid virus and the ACE2 receptor is analogous to a lock-and-key mechanism, allowing the virus to enter host cells. The ACE2 complex is inhibited by the following non-pharmaceutical phytochemicals: quercetin, resveratrol, ginseng, naringenin, hesperetin, ashwagandha, baicalein, glycyrrhizin, artemisinin, and luteolin.

The ACE2 receptor is also down-regulated by zinc acting in concert with the following non-pharmaceutical phytochemicals: chlorogenic acid, EGCG, gallic acid, luteolin, quercetin, resveratrol and tannic acid.

The TMPRSS2 protein plays a critical role in Covid. It facilitates viral entry into human cells by cleaving and activating Covid's spike(S) protein, which allows for membrane fusion between the virus and the host cell. This process enables the virus to enter the cell directly at the plasma membrane, independent of endosomal pathways involving cathepsins. TMPRSS2 is inhibited by: lactoferrin, ashwagandha, quercetin, myricetin, naringin, mangiferin, baicalein and hesperetin.

The furin cleavage site (FCS) plays a critical role in viral entry and pathogenesis of Covid. It is a unique insertion at the junction of the S1 and S2 subunits of the spike protein, which is not present in other closely related sarbecoviruses. This site allows the host protease furin to cleave the spike protein during viral assembly, priming it for activation. Cleavage at this site facilitates a conformational change in the spike protein, promoting an “open” state that enhances binding to the ACE2 receptor on human cells, thereby increasing viral transmissibility. While the FCS significantly enhances transmissibility, recent evidence suggests it is not absolutely required for transmission, as FCS-disrupted mutants can still spread in direct contact settings, albeit with reduced efficiency compared to wild-type virus. The furin protease is inhibited by: fucoidan, ashwagandha, andrographolide, quercetin, resveratrol and luteolin.

The CD147 receptor is thought to play a significant role in Covid infections. It functions as a potential alternative receptor to ACE2, facilitating viral entry into host cells by interacting with the virus's spike protein. The CD147 receptor is inhibited by: myricetin and resveratrol.

The RAGE/HMGB1 receptor axis plays a significant role in amplifying inflammation and tissue damage during Covid. HMGB1, a damage-associated molecular pattern (DAMP) molecule, is released from dying or stressed cells and can bind to the RAGE receptor, which is highly expressed in lung tissue, particularly on type I pneumocytes, alveolar macrophages, and endothelial cells. This interaction activates pro-inflammatory signaling pathways such as NF-κB and MAPK, leading to the production of cytokines and reactive oxygen species (ROS), thereby contributing to the cytokine storm and endothelial dysfunction observed in severe cases. The RAGE/HMGB1 Receptor Axis is inhibited by: glycyrrhizin, EGCG, chlorogenic acid and quercetin.

There are three endocytosis-related modes of viral entry via: (1) clathrin; (2) cathepsin L and (3) tumor necrosis factor-α-converting enzyme (ADAM17).

Endocytosis plays a significant role in Covid infections. After the virus binds to the ACE2 receptor on the host cell surface via its S glycoprotein, it can enter the cell through endocytosis. This process involves the internalization of the entire virus into an endosome, where the acidic environment and proteases such as cathepsin L facilitate viral membrane fusion with the endosomal membrane, allowing the viral RNA to be released into the cytosol. These non-pharmaceutical phytochemicals inhibit endocytosis: oleuropein, silibinin and ginseng.

Clathrin plays a critical role in the viral entry of Covid by mediating endocytosis. The virus uses its spike glycoprotein to bind to the host cell surface receptor ACE2, after which it undergoes rapid internalization via clathrin-mediated endocytosis. This process involves the formation of clathrin-coated pits on the plasma membrane that engulf the virus, leading to its internalization into endosomal compartments. These non-pharmaceutical phytochemicals inhibit clathrin: cinnamon and EGCG.

Cathepsin L (CatL) plays a critical role in the endosomal entry pathway of Covid. After the virus binds to the ACE2 receptor on the host cell surface, it is internalized via endocytosis into endosomes. Within the acidic environment of the endosome, CatL cleaves the viral S glycoprotein at the S1-S2 boundary which induces conformational changes necessary for membrane fusion. This proteolytic activation by CatL allows fusion between the viral envelope and the endosomal membrane, facilitating the release of the viral genome into the host cell cytoplasm. These non-pharmaceutical phytochemicals inhibit CatL: ursolic acid, thymoquinone, artemisinin, myricetin and quercetin.

ADAM17 plays a significant role in facilitating entry of Covid into host cells via endocytosis. It enhances viral infectivity by acting in the lysosomal entry pathway, either at the plasma membrane just before endocytosis or after the virus has been internalized. ADAM17 contributes to the priming of the S protein, an essential step for viral entry, through its cleavage activity. This protease can cleave the S protein, promoting fusion likely after endocytosis. Inhibition of ADAM17 reduces both viral uptake and fusion. These non-pharmaceutical phytochemicals inhibit ADAM17: silibinin and curcumin.

There are four targets for viral replication inhibition and blockade: (1) 3-chymotrypsin-like proteases (3CLpro) also known the main proteases (Mpro); (2) papain-like protease (PLpro); (3) helicase and (4) RNA-dependent RNA polymerase (RdRp).

3CLPro/MPro inhibition disrupts the virus's ability to replicate and assemble new viral particles. The 3CLpro enzyme is responsible for cleaving the viral polyproteins into functional non-structural proteins essential for viral replication and transcription. By inhibiting 3CLpro, this processing is blocked, effectively halting the viral life cycle. These non-pharmaceutical phytochemicals inhibit 3CLPro: hesperidin, glycyrrhizin, luteolin, andrographolide, naringenin, curcumin, baicalein and myricetin.

Inhibition of PLpro disrupts viral replication and helps restore the host's immune response during Covid infections. PLpro is essential for processing the viral polyproteins, specifically cleaving the N-terminus of the replicase polyprotein to release non-structural proteins (Nsp1, Nsp2, and Nsp3) required for viral replication. By inhibiting PLpro, this cleavage is blocked, thereby limiting the virus's ability to replicate. These non-pharmaceutical phytochemicals inhibit PLpro: ginkgo biloba, curcumin, quercetin and baicalein.

Helicase inhibition disrupts the function of the Covid nonstructural protein 13(Nsp 13), a helicase essential for viral replication. This enzyme unwinds double-stranded RNA during replication, resolves RNA secondary structures like G-quadruplexes, and removes RNA-bound proteins that could impede replication. By inhibiting Nsp13, the viral replication-transcription complex (RTC) is impaired, thereby reducing the amplification of viral particles. These non-pharmaceutical phytochemicals inhibit helicase: ferulic acid, gallic acid, resveratrol, luteolin, myricetin, quercetin and baicalein.

RdRp inhibition blocks the replication of Covid by targeting a key enzyme the virus uses to copy its genetic material. The viral RdRp is essential for synthesizing new RNA strands from the viral RNA template, a critical step in the virus's life cycle. Inhibiting this enzyme prevents the virus from multiplying within host cells, thereby reducing viral load and potentially mitigating disease progression. These non-pharmaceutical phytochemicals inhibit RdRp: vitamin B12, silibinin, hesperidin, quercetin and berberine.

Spike protein attenuation occurs by the enzymatic degradation of spike. The enzymatic degradation of the Covid spike protein can be facilitated by nattokinase, an enzyme derived from fermented soybeans. Nattokinase has demonstrated the ability to degrade the spike protein in a time- and dose-dependent manner suggesting its potential role in breaking down the protein.

Covid induces significant platelet dysfunction, contributing to the immunothrombotic complications seen in Covid. Platelets in infected individuals exhibit a hyperactive phenotype characterized by increased expression of tissue factor (TF), enhanced release of extracellular vesicles, and elevated formation of platelet-leukocyte aggregates. This activation is driven by multiple mechanisms, including direct and indirect interactions with the virus. Covid can bind to platelets via its spike protein, potentially stimulating them to release inflammatory and procoagulant factors, thereby promoting leukocyte-platelet aggregation. These non-pharmaceutical phytochemicals and micronutrients inhibit Covid induced platelet dysfunction: ginseng, grape seed extract, quercetin, ginkgo biloba and zinc.

Host defense peptides (HDPs), also known as antimicrobial peptides (AMPs), play a multifaceted role in the immune response to Covid. They act as a first line of defense by directly inhibiting viral entry and replication; for example, human defensin 5(HD 5) and lactoferrin can block the ACE-2 receptor, preventing viral attachment. Beyond direct antiviral effects, HDPs modulate the immune response by regulating inflammation—dampening key pro-inflammatory molecules like TNF-α, IL-1β, and IL-6, which may help mitigate cytokine release syndrome (CRS) associated with cytokine storm. Host defense peptides are activated by vitamin D and by butyrate.

SPMs derived from omega-3 fatty acids like eicosapentaenoic acid (EPA) and docosahexaenoic acid (DHA), play a crucial role in modulating the inflammatory response in severe cases of Covid. Resolvins are SPM's which help resolve the hyperinflammation associated with cytokine storm by actively turning off the inflammatory process, thereby limiting multi-organ failure and death.

Tetherin (BST-2) increases cell sensing by acting as a viral sensor which activates cellular defenses against invading viruses. It also acts as a host restriction factor against Covid by tethering nascent viral particles to the surface of infected cells, thereby inhibiting their release and spread to neighboring cells. Tetherin is stimulated by type I interferon which the method rapidly induces.

Innate lymphoid cells (ILCs) play a role in the early immune response to Covid, with evidence suggesting that pre-activated tissue-specific immunity mediated by ILCs may influence disease outcomes in Covid. Circulating ILCs are significantly reduced in number in Covid patients, both in percentage and absolute counts, indicating their recruitment to infected tissues or differentiation at sites of infection. These micronutrients and non-pharmaceutical phytochemicals up-regulate ILC's: vitamin A, vitamin D, vitamin C, vitamin E, zinc, curcumin, ginseng and Lactobacillus paracasei postbiotic.

Non-structural protein 1 (Nsp1) is a major virulence factor in Covid and plays a central role in suppressing the host's innate immune response. It achieves this by blocking the entry of host mRNA into the ribosome, thereby inhibiting translation of cellular proteins and inducing degradation of host mRNA. This host shutoff mechanism particularly suppresses the production of type I interferon (IFN), a critical component of the antiviral immune response, allowing the virus to evade detection and propagate efficiently. Notably, while Nsp1 inhibits host protein synthesis, it does not block the translation of viral mRNA, giving the virus a replicative advantage. Mutations or loss of Nsp1 function result in a stronger IFN response and reduced viral propagation, both in vitro and in vivo, highlighting its essential role in pathogenesis. Due to its pivotal role in immune evasion, Nsp1 is considered a key target to promote cellular defenses. These non-pharmaceutical phytochemicals inhibit NSP1: glycyrrhizic acid, ashwagandha and boswellic acid.

Continuous activation of cellular defenses against hijacking includes: (1) renin-angiotensin-aldosterone regulation; (2) Type I interferons (IFN-I) pre-infection priming; (3) complement dysregulation inhibition; (4) energy hijacking mitigation; (5) mast cell stabilization; (6) neutrophil extracellular trap inhibition (NETs); (7) neutrophil elastase inhibition; (8) macrophage activation syndrome inhibition; (9) thrombin/thrombomodulin regulation; (10) glucose-regulated protein 78 (GRP 78)/heat shock protein 5(HSP 5) regulation; (11) iron dysregulation mitigation and (12) syncytia formation inhibition.

The renin-angiotensin-aldosterone system (RAAS) plays a critical role in the pathogenesis of Covid, primarily through the interaction of the Covid virus with the ACE2 receptor which serves as the principal cellular receptor for viral entry. Upon infection, Covid downregulates ACE2 expression on cell surfaces, disrupting the normal balance of the RAAS. ACE2 normally converts angiotensin II (Ang II), a peptide associated with vasoconstriction, inflammation, and fibrosis, into angiotensin-(1-7), which has vasodilatory, anti-inflammatory, and protective effects. The downregulation of ACE2 leads to reduced breakdown of Ang II and diminished production of protective angiotensins, resulting in an accumulation of Ang II and a shift toward a pro-inflammatory, pro-thrombotic, and a vasoconstrictive state. This imbalance may contribute to the development of acute respiratory distress syndrome (ARDS), myocardial injury, and multi-organ dysfunction observed in severe Covid cases. Overactivation of the RAAS due to ACE2 deficiency can exacerbate lung injury and cardiovascular complications, suggesting that RAAS dysregulation is a key driver of severe disease. Individuals with pre-existing conditions such as hypertension, diabetes, or cardiovascular disease often have underlying RAAS imbalances and reduced ACE2 activity, which may partly explain their increased risk of severe outcomes from Covid. These non-pharmaceutical phytochemicals mitigate the dysregulation of the RAAS: quercetin, luteolin, EGCG, chlorogenic acid, hesperidin, naringenin, baicalein, andrographolide, myricetin, glycyrrhizin, curcumin, ginkgo biloba and ursolic acid.

IFN-I play a critical role as the first line of defense against Covid infection by initiating many antiviral cellular defenses. IFN-I are produced primarily by plasmacytoid dendritic cells in the gut, triggering the JAK-STAT signaling pathway, which leads to the up-regulation of hundreds of interferon-stimulated genes (ISGs) that establish an antiviral state in neighboring cells. This includes inhibiting viral mRNA translation, degrading viral RNA, and up-regulating transmembrane proteins like IFITM3 that prevent viral fusion and release into the cytoplasm, thereby limiting viral spread. The timing of IFN-I response is crucial: an early, transient peak in IFN-I expression is associated with mild or asymptomatic disease, effective viral clearance, and rapid activation of Covid-specific CD8+ T cells. In contrast, delayed or impaired IFN-I signaling is linked to severe disease, higher viral loads, and a dysregulated immune response characterized by excessive inflammation and cytokine storm. This deficiency can result from genetic factors, autoantibodies against IFN-I, or viral evasion mechanisms that suppress IFN production. These non-pharmaceutical phytochemicals, micronutrients and postbiotics enable safe pre-infection priming of IFN-I: lacticaseibacillus paracasei postbiotic directly activates plasmacytoid dendritic cells, vitamin D (also induces plasmacytoid dendritic cells by creating synergies with postbiotic stimulation), glycyrrhizin, zinc, ferulic acid, lipoic acid, selenium, N-acetyl cysteine, glucosamine, beta glucan and elderberry.

Complement dysregulation plays a significant role in the development of the cytokine storm observed in severe Covid. Activation of the complement system generates the anaphylatoxins C3a and C5a, which are potent mediators of inflammation and capable of activating various immune cells including neutrophils, monocytes, macrophages, and T cells. These activated cells, particularly macrophages and neutrophils, produce high levels of proinflammatory cytokines such as TNF-α, IL-1β, and IL-6, contributing to the hyperinflammatory state known as the cytokine storm. Elevated plasma levels of C5a and IL-6 have been observed in patients with moderate to severe Covid, supporting this link. This overactivation is further amplified by a feedback loop involving NETs, which contain complement components that stabilize the AP C3 convertase, leading to more C3a and C5a production and further neutrophil recruitment and activation. Pre-infection priming with these non-pharmaceutical natural products mitigate the risk of complement dysregulation: vitamin D, baicalein, andrographolide, ginseng, fucoidan, boswellic acid and artemisinin.

Covid hijacks cellular energy by disrupting the host cell's metabolic processes, particularly targeting mitochondria and lipid metabolism to fuel its replication. The virus interferes with mitochondrial function, which are the cell's primary energy-producing structures, by inhibiting genes responsible for producing proteins involved in energy production. This disruption forces cells to rely on alternative metabolic pathways, which the virus then exploits to generate more copies of itself. Mitochondrial dysfunction persists in organs such as the heart, liver, and kidneys even after the acute infection, suggesting a role in long-term damage and conditions like long Covid. Pre-infection priming with these non-pharmaceutical phytochemicals helps to prevent hijacking of cellular energy by pathogens: nicotinamide riboside, pterostilbene, quercetin, berberine and curcumin.

Mast cell activation and degranulation caused by pathogens produce profound releases of cytokines and chemokines. These actions contribute to vascular leakage, increased permeability, and tissue damage observed in severe cases, thereby playing a significant role in the progression of cytokine storm. Pre-infection priming with these non-pharmaceutical phytochemicals helps to stabilize mast cells against activation and degranulation: vitamin D, luteolin, quercetin, EGCG, silibinin, cinnamon, honokiol and ellagic acid.

NETs play a significant role in the development and amplification of cytokine storm. NET's are characterized by the excessive and uncontrolled release of pro-inflammatory cytokines. NETs contribute to this hyperinflammatory state by directly promoting inflammation, thrombosis, and tissue injury. They act as a platform for the activation of other immune cells; for example, NETs can activate plasmacytoid dendritic cells via TLR9, stimulating the production of type I interferons, and can directly lower the activation threshold of T cells, thereby amplifying the immune response. Furthermore, NETs are induced by key cytokines involved in cytokine storm, such as IL-1β, IL-8, TNF-α, and IFN-γ, creating a vicious cycle where cytokines stimulate NET formation, and NETs, in turn, perpetuate inflammation and coagulation. This dysregulated interaction is particularly evident in severe conditions like Covid and sepsis, where elevated NET levels correlate with poor outcomes, including acute respiratory distress syndrome (ARDS) and multi-organ dysfunction. Pre-infection priming with these non-pharmaceutical phytochemicals mitigate the risk of NET's: vitamin D, N-acetyl cysteine, resveratrol, ginger, ashwagandha, amla, quercetin, andrographolide, EGCG, curcumin, tinospora cordifolia, piperine, cinnamon, glycyrrhizin, complement inhibition and IL-8 Inhibition induced by garlic, luteolin, quercetin, ashwagandha and curcumin.

Neutrophil elastase (NE) plays a significant and complex role in the development and regulation of cytokine storm, particularly in severe infections and inflammatory conditions. It contributes to the hyperinflammatory state by directly modulating cytokine expression and amplifying immune responses. In the context of severe infections like pseudomonas aeruginosa pneumonia, NE is essential for mounting an effective host defense, as its absence leads to reduced cytokine levels and increased mortality, indicating a protective role in initiating an appropriate inflammatory response. However, in pathological conditions such as severe Covid, the excessive release of NE from activated neutrophils contributes to the cytokine storm. NE promotes the formation of NETs, which are associated with acute lung injury and ARDS. The release of NE, along with ROS and activation of the NF-κB pathway, exacerbates the cytokine storm, leading to vascular thrombosis, hypoxia, and multiple organ failure. Pre-infection priming with these non-pharmaceutical phytochemicals can safely mitigate the risk of dysregulated NE: luteolin, baicalin, quercetin, myricetin, naringenin, EGCG, chlorogenic acid, ferulic acid, cinnamon and thymoquinone.

Macrophage activation syndrome (MAS) is a life-threatening hyperinflammatory condition associated with severe cases of Covid, contributing to the cytokine storm that can lead to ARDS, multi-organ failure, and death. Covid triggers MAS by infecting pneumocytes and macrophages, inducing pyroptosis and activating inflammatory pathways involving cytokines such as IL-6, TNFα, and IL-1, which create a feedback loop between macrophages and T cells, amplifying systemic inflammation. Pre-infection priming with these non-pharmaceutical nutritional products can safely mitigate the risk of MAS: vitamin D, ellagic acid, gallic acid, moringa, berberine, dihydromyricetin, ginseng, omega-3 fatty acids, curcumin, resveratrol and hydroxytyrosol.

Thrombomodulin (TM) is a glycoprotein expressed on the surface of endothelial cells that plays a critical role in maintaining anticoagulant and anti-inflammatory states. The inflammatory response in severe Covid triggers the release of angiopoietin-2 (Ang2), which binds competitively to TM and inhibits its anticoagulant function, thereby promoting a prothrombotic state. This interaction provides a mechanistic link between inflammation and coagulopathy in Covid. Vitamin D plays a crucial role in regulating thrombomodulin expression, thereby contributing to the prevention of blood clot formation and maintaining anticoagulant homeostasis.

GRP78 is a heat shock protein (HSP) that plays a significant role in the cellular response to Covid. It is a stress-inducible molecular chaperone primarily located in the endoplasmic reticulum (ER). Molecular chaperones are involved in protein folding, prevention of protein aggregation, and cellular stress responses, and their expression increases under adverse conditions such as viral infection. The Covid virus cannot produce its own chaperones and therefore hijacks the host's heat shock proteins to assist in the proper folding of its viral proteins which are essential for viral replication. This reliance on host HSPs makes them critical for the virus's ability to grow and proliferate within human cells. Pre-infection priming with these non-pharmaceutical phytochemicals may help block viral entry by inhibiting HSP's: EGCG, hesperidin, curcumin, hydroxytyrosol, chlorogenic acid, salidroside, honokiol, mangiferin, luteolin, capsaicin, berberine, naringin, diosmin and thymoquinone.

Iron dysregulation plays a significant role in both acute and long-term outcomes of Covid, affecting immune function, oxygen transport, and inflammation. During infection, the body's immune response increases levels of hepcidin, the main regulator of iron homeostasis, which reduces plasma iron availability by blocking its release from macrophages—a defense mechanism to limit iron access to pathogens. Iron chelators work by scavenging excess iron and reducing ferritin levels, thereby decreasing the availability of iron for viral replication. This mechanism is particularly relevant in Covid as iron is essential for viral RNA synthesis and replication processes. Additionally, iron chelation has been shown to suppress endothelial inflammation, a key pathophysiological mechanism behind systemic organ involvement in Covid, by inhibiting IL-6 synthesis through the downregulation of NF-κB. These non-pharmaceutical compounds are naturally occurring iron chelators: lactoferrin, grape seed extract, curcumin, EGCG, quercetin, cyanidin and ferulic acid.

Syncytia play a significant role in the severity and progression of Covid by facilitating viral spread and immune evasion. They form when Covid-infected cells express the S protein on their surface, which binds to ACE2 receptors on neighboring cells, triggering membrane fusion and creating multinucleated giant cells. This cell-to-cell fusion allows the virus to disseminate without being exposed to the extracellular environment, thereby avoiding neutralizing antibodies and interferon responses, which contributes to immune evasion. Syncytia are associated with severe disease and lung damage. Natural compounds inhibit syncytia formation by inhibition of anoctamin-1 (TMEM16) proteins. These natural compounds are: luteolin, quercetin, allicin and silibinin. Syncytia are also inhibited by blocking TMPRSS2 via lactoferrin.

Continuous activation of cellular defenses against cellular injury includes: (1) rebalancing; (2) restoring and maintaining continuous tumor surveillance and cellular repair and (3) pre-infection priming to protect specific organs.

Rebalancing is an outcome of pre-infection priming. Rebalancing prevents cellular self-destruction by influencing how T-cells respond to pathogen-induced molecular warfare. This occurs by shifting the cellular response of Th1/Th17/Treg cells towards an anti-inflammatory versus pro-inflammatory phenotype. Rebalancing is achieved naturally through pre-infection priming with amino acids and non-pharmaceutical phytochemicals that mitigate hyperinflammatory tissue destruction by restoring the adequacy of cellular nutrients. The natural compounds which support rebalancing are: glutamine, beta glucan, butyrate, leucine, serine, methionine, vitamin A, vitamin B5, baicalein, curcumin, mangiferin, andrographolide, hesperidin, quercetin, EGCG and resveratrol.

Restoring and maintaining continuous tumor surveillance and cellular repair are comprised of: (1) reactivating tumor suppressor genes which are inactivated by pathogens and (2) restoring and maintaining DNA repair functions which are inhibited by pathogens.

Covid infections may lead to the down regulation of key tumor suppressor genes, specifically p53 and pRB, through the action of viral proteins. The Covid non-structural protein 15 (Nsp15) is highly conserved and likely interacts with and promotes the degradation of pRB, similar to mechanisms observed in other viruses. Additionally, the Covid protein Nsp3 may increase the degradation of p53 by stabilizing the E3 ubiquitin ligase RCHY1, which targets p53 for proteasomal degradation. Both p53 and pRB are critical tumor suppressors, and their inhibition is a known mechanism in viral oncogenesis. Pre-infection priming with these non-pharmaceutical phytochemicals may help block Covid inhibition of tumor suppressor genes: vitamin D, thymoquinone, EGCG, resveratrol, carnosic acid, curcumin and honokiol.

Covid can inhibit DNA repair functions through multiple mechanisms. The virus appears to interfere with key proteins involved in DNA repair, such as 53BP1, which is essential for repairing DNA double-strand breaks; viral proteins impair its function, leading to the accumulation of DNA damage. Nsp1 also interferes with DNA polymerase α, further disrupting DNA synthesis and repair processes. Covid infections also lead to a reduction in deoxynucleoside triphosphates (dNTPs), the building blocks of DNA, by redirecting cellular resources toward viral RNA synthesis using ribonucleoside triphosphates (rNTPs), thereby limiting the availability of materials needed for DNA repair and replication. This disruption, combined with virus-induced oxidative stress and inflammation, contributes to genomic instability. Pre-infection priming with these non-pharmaceutical phytochemicals may help restore normal cellular DNA repair functions: vitamin D, curcumin, quercetin, resveratrol, ellagic acid, honokiol, ferulic acid, thymoquinone, capsaicin, berberine and EGCG.

Pre-infection priming to protect specific organs and tissues is comprised of protecting: (1) brain; (2) lungs; (3) liver; (4) heart; (5) kidneys and (6) endothelial-mesenchymal transitions.

The persistence of the Covid spike protein in the skull-meninges-brain axis long after viral clearance has been associated with dysregulated inflammatory pathways and neurodegeneration-related changes. Proteomic analysis revealed that spike protein-positive brain regions show upregulation of GFAP, a biomarker for blood-brain barrier damage, and dysregulation of proteins linked to neurological diseases. This indicates that the presence of spike protein can alter the brain's proteome, potentially overwhelming normal protein clearance systems such as the ubiquitin-proteasome system or autophagy.

Brain protection achieved through pre-infection priming is comprised of protections involving: (1) ubiquitin proteasome system; (2) unfolded protein response; (3) chaperones; (4) amyloid deposition; (5) autophagy and (6) hippocampal, neurogenesis upregulation.

Pre-infection priming with these non-pharmaceutical phytochemicals may help restore the ubiquitin proteasome system in the brain: quercetin, resveratrol and oleuropein.

Pre-infection priming with these non-pharmaceutical phytochemicals may help restore the unfolded protein response system in the brain: vitamin D, curcumin, resveratrol. EGCG, quercetin, oleuropein, baicalein, berberine, ginseng, piperine, honokiol, ursolic acid, carnosic acid, sulforaphane and artemisinin.

Molecular chaperones in the brain are proteins that play a critical role in maintaining protein homeostasis (proteostasis), which is essential for the proper functioning of the proteome and cellular health. They are often referred to as the “guardians of proteins,” analogous to how p53 is the guardian of the genome. These chaperones assist in the proper folding, assembly, and stabilization of other proteins, preventing misfolding and aggregation, and facilitating the degradation of terminally misfolded proteins. They are particularly vital in neurons, which are postmitotic and have long extensions, making them highly sensitive to the accumulation of misfolded proteins, especially with aging. There is evidence suggesting that Covid may impact molecular chaperones in the brain, particularly the endoplasmic reticulum (ER) chaperone sigma-1 receptor. The sigma-1 receptor plays a role in modulating ER stress-related proteins, including X-box-binding protein 1 (XBP-1), which has been implicated in the reactivation of viruses such as Epstein-Barr virus (EBV) during long Covid. Given that Covid infection can trigger significant cellular stress and neuroinflammation, the function of chaperones like the sigma-1 receptor may be altered as part of the brain's response to infection and stress.

Pre-infection priming with these non-pharmaceutical phytochemicals may help restore chaperones in the brain: curcumin, glycyrrhizin and ashwagandha.

Evidence suggests that Covid infections can lead to amyloid deposition in the brain. Studies have shown that the virus can enter the brain through the olfactory neurons or a disrupted blood-brain barrier (BBB), leading to the accumulation of amyloid-beta (Aβ) deposits, which are associated with Alzheimer's disease (AD) pathology. The virus stimulates the production of amyloids both extracellularly, through spike protein interactions with Aβ, and intracellularly, via structural and accessory viral proteins forming Covid amyloids. Infection with Covid has been linked to increased expression of Alzheimer's-related mediators, Aβ deposition, and neuroinflammation in human induced pluripotent stem cell (iPSC)-derived neurons and in brain organoids. Postmortem analyses of young Covid patients with autism revealed Aβ deposition and neuroinflammation in cognitive brain regions. Furthermore, transgenic mouse models of AD infected mice with Covid showed accelerated amyloid deposition and neuroinflammation compared to uninfected controls, suggesting the virus can induce or exacerbate amyloid pathology, especially in predisposed individuals.

Vitamin D appears to inhibit amyloid deposition in the brain through multiple mechanisms. Studies in transgenic mouse models of AD show that a vitamin D-enriched diet significantly reduces the number and size of amyloid plaques compared to vitamin D-deficient or control diets. This reduction is associated with decreased levels of Aβ peptides, particularly Aβ40 and Aβ42, in the brain. Vitamin D exerts its effects by modulating the processing of the amyloid precursor protein (APP). It decreases the activity of β-secretase (BACE1), a key enzyme responsible for initiating Aβ production, by reducing BACE1 gene expression and protein levels. It also reduces γ-secretase activity, which is involved in the final cleavage step that releases Aβ peptides. Furthermore, vitamin D enhances the non-amyloidogenic pathway by increasing α-secretase activity in some studies, which prevents Aβ formation.

In addition to vitamin D, pre-infection priming with these non-pharmaceutical phytochemicals may act synergistically with vitamin D to prevent amyloid deposition in the brain: punicalagin, ellagic acid, curcumin, ferulic acid, EGCG, myricetin, oleuropein and ursolic acid.

Covid can impair autophagy in the brain. The Covid protein ORF3a has been shown to block autophagic flux in the brain, leading to the accumulation of proteins associated with neurodegeneration such as α-synuclein and glycosphingolipids. This disruption occurs because ORF3a inhibits autophagosome-lysosome (A-L) fusion by interacting with the host protein VPS39 and the HOPS complex, preventing the final degradation step in the autophagy-lysosome pathway (ALP). While some viral proteins like ORF8 and nucleocapsid protein may initially activate autophagy by inhibiting mTORC1, the overall effect of Covid is a dysregulation of autophagy due to impaired maturation of autophagosomes. This autophagy dysfunction in neurons may contribute to neurological manifestations of Covid, including “brain fog” seen in long-Covid. Neurons are particularly vulnerable to such disruptions because they rely heavily on autophagy for clearing damaged components and are unable to self-renew.

Vitamin D, specifically its active metabolite calcitriol, can restore autophagy in the brain, particularly following injury. Studies in rat models of traumatic brain injury (TBI) demonstrate that calcitriol treatment activates the VDR and restores autophagy flux in the cortex region of the brain, which is impaired after injury. This restoration is evidenced by a decrease in the LC3II/LC3I ratio and reduced p62 protein levels, indicating improved autophagosome clearance. The protective effects of calcitriol are associated with this restoration of autophagy flux and a subsequent reduction in apoptosis. Furthermore, vitamin D signaling is known to regulate autophagy at multiple levels, including induction, nucleation, and degradation, by modulating factors like mTOR, Beclin 1, and lysosomal activity. Research also suggests that vitamin D deficiency is linked to diseases involving defective autophagy, and supplementation can help restore autophagic balance.

Pre-infection priming with these non-pharmaceutical phytochemicals may also help to normalize autophagy in the brain: quercetin, resveratrol, oleuropein, spermidine, sulforaphane and berberine.

Covid may impair hippocampal neurogenesis. The hippocampus, a brain region critical for memory consolidation, learning, and neurogenesis—particularly in the dentate gyrus—is vulnerable to the effects of Covid infection. Studies indicate that the virus may target this region, contributing to memory loss through mechanisms such as oxidative stress, cytokine-driven inflammation, and vascular damage. Reduced hippocampal neurogenesis has been proposed as a potential cause of memory loss in long Covid patients. Furthermore, Covid infection has been associated with structural brain changes, including reduced volume in limbic areas like the parahippocampal gyrus, and may attenuate endogenous neural stem cell activity, which is essential for neurogenesis. Impairment in neurogenesis could underlie cognitive deficits and olfactory dysfunction observed in Covid patients, and may represent a neuropathological link to neurodegenerative processes.

Vitamin D appears to play a protective role against hippocampal damage and supports neurogenesis, which may be relevant in the context of Covid related neurological complications. VDR are expressed in the hippocampus, and the active form of vitamin D, 1,25(OH)2D3, has been shown to regulate neurotrophic factors such as nerve growth factor (NGF) and brain-derived neurotrophic factor (BDNF), which are essential for neuronal survival, differentiation, and synaptic plasticity. These mechanisms support hippocampal neurogenesis and function. In the context of Covid, neuroinflammation and oxidative stress can lead to hippocampal dysfunction and cognitive deficits, partly due to cytokine storm and inflammatory mediators like IL-6 and TNF-α crossing the blood-brain barrier and affecting the hippocampus. Vitamin D has anti-inflammatory and immunomodulatory properties that can suppress these pro-inflammatory cytokines, potentially reducing neuroinflammation and protecting hippocampal neurons. Additionally, vitamin D enhances antioxidant defenses, such as glutathione and superoxide dismutase, which help mitigate oxidative stress-induced neuronal damage in the hippocampus. Furthermore, vitamin D deficiency has been linked to cognitive decline and increased risk of neurodegenerative conditions, while supplementation has been associated with improved memory and synaptic function in aging models. Given that Covid may disrupt the renin-angiotensin system and reduce ACE2 activity—processes modulated by vitamin D—its neuroprotective role could extend to maintaining hippocampal integrity post-infection.

Pre-infection priming with these non-pharmaceutical compounds helps normalize hippocampal neurogenesis: lithium, curcumin, thymoquinone, ginseng, silymarin, baicalein, ferulic acid, hesperidin, berberine, ginkgo biloba, punicalagin and ellagic acid.

Lung injuries by pathogens are mitigated by two types of immune protections: (1) pre-deployment of tissue specific immunity in the lungs against molecular attacks by pathogens and (2) prevention of lung fibrosis following infections.

Tissue-specific immunity in the lungs involves localized immune responses that can both protect against and contribute to injury during Covid infection. Resident immune cells, such as alveolar macrophages and tissue-resident T cells, play critical roles in early viral detection and containment. For instance, interferon-stimulated genes like ACE2 are upregulated in airway epithelial cells upon viral detection, which may initially serve a protective function by modulating lung injury responses, although this can also inadvertently facilitate viral entry. Tissue-specific immunity protects the lungs by coordinating targeted antiviral defenses through resident and recruited immune cells, but when dysregulated, it can lead to immunopathology. The spatial and temporal control of these responses—such as the balance between interferon-mediated protection and its potential exploitation by the virus—is crucial in determining disease outcome.

Vitamin D contributes to tissue-specific immune protection against lung injury from Covid by enhancing the barrier function of the lung lining and modulating the immune response. Vitamin D strengthens the lung epithelial barrier, reducing the ability of Covid and other viruses to penetrate the airways and potentially decreasing fluid leakage that leads to pneumonia. It also plays a role in regulating the renin-angiotensin system by increasing ACE2 levels, which may protect against acute lung injury, as decreased ACE2 is associated with worse outcomes in respiratory infections like Covid. Additionally, vitamin D exerts immunomodulatory effects by promoting an antiviral state in lung tissue. It upregulates antimicrobial peptides and enhances type I interferon responses, which are critical for early viral defense. In mouse models, a vitamin D-rich diet reduced lung viral titers and pro-inflammatory cytokines like TNF, IL-6, and IL-1β, while boosting protective immune responses. The active form of vitamin D, calcitriol, acts locally in lung epithelial cells that express the VDR, enabling autocrine and paracrine signaling that supports anti-inflammatory and antimicrobial functions within the respiratory tract.

Pre-infection priming with these non-pharmaceutical compounds help to enhance tissue specific immunity in the lungs: astragalus, fucoxanthin, ergothioneine and baicalein.

Covid can induce lung fibrosis, particularly in severe cases. Covid infection triggers profibrotic macrophage responses and leads to pronounced fibroproliferative ARDS, which is associated with clinical, radiographic, histopathological, and ultrastructural hallmarks of pulmonary fibrosis. The virus also causes direct alveolar damage and induces a hyperinflammatory state, leading to chronic inflammation, epithelial damage, fibroblast activation, and excessive deposition of collagen and other extracellular matrix proteins, resulting in alveolar scarring and fibrosis. This process is driven in part by the overexpression of pro-fibrotic cytokines and growth factors such as transforming growth factor β1 (TGF-β1), interleukin-6 (IL-6), and tumor necrosis factor α (TNF-α). Covid may also directly promote fibrosis through mechanisms such as enhancing TGF-β signaling via its nucleocapsid protein.

Vitamin D may mitigate the risk of lung fibrosis, potentially through mechanisms that counteract the effects of pathogen-induced lung injury. Vitamin D deficiency is strongly associated with various lung diseases, including those exacerbated by pathogens, and low levels are linked to impaired lung function and increased disease severity. Vitamin D exerts protective effects by modulating key pathways involved in fibrosis development. It can inhibit the TGF-β signaling pathway, which is a central driver of fibrosis and is activated during lung injury from various causes, including infections. Vitamin D suppresses the epithelial-mesenchymal transition (EMT), a process critical in fibrosis, particularly when induced by TGF-β1. Furthermore, vitamin D has been shown to inhibit the activation of RAAS in the lung, a pathway implicated in fibrosis that can be triggered by injury, including that from pathogens. Studies in animal models demonstrate that vitamin D supplementation can prevent or reduce bleomycin-induced pulmonary fibrosis, a model often used to study fibrotic lung disease. This protective effect is associated with reduced collagen deposition, decreased expression of fibrotic markers like α-smooth muscle actin and fibronectin, and improved lung function. While these findings suggest a broad antifibrotic role for vitamin D, the evidence specifically linking it to mitigating fibrosis caused by pathogens is indirect, relying on its ability to regulate the inflammatory and fibrotic responses that follow pathogen-induced lung damage.

Pre-infection priming with these non-pharmaceutical phytochemicals have also been shown to mitigate the risks of lung fibrosis following molecular attack by pathogens: vitamin C, salidroside, thymoquinone and curcumin.

Covid can induce liver injury, although the exact mechanisms are not fully understood and may involve multiple pathways. Liver dysfunction has been observed in 21.5% to 45.71% of patients with Covid infection, particularly those with severe disease. Elevated liver enzymes such as alanine aminotransferase (ALT) and aspartate aminotransferase (AST) were found in about 20% to 43.4% of patients, with higher rates in severe cases. The potential mechanisms of liver injury include: systemic inflammation and cytokine storm, hypoxia and ischemia, endothelial dysfunction, thrombosis and direct viral effects.

Pre-infection priming with these non-pharmaceutical phytochemicals have been shown to mitigate the risks of liver fibrosis following molecular attack by pathogens: R-α-lipoic acid, salidroside, baicalein, silibinin, hesperidin, naringenin and quercetin.

Cardiac injuries induced by Covid include myocarditis, pericarditis, ferroptosis, acute heart failure and cardiac fibrosis including fibrosis of cardiac electrical conduction fibers contributing to cardiac arrhythmias. Mechanisms contributing to these injuries involve direct viral effects via ACE-2 receptor binding, cytokine storm, endothelial dysfunction, microvascular thrombosis, systemic inflammation, and direct injury by the Covid spike protein.

Pre-infection priming with these non-pharmaceutical phytochemicals mitigate the risks of cardiac ferroptosis following molecular attack by pathogens: salidroside, baicalin, quercetin, naringenin, hesperidin, curcumin, carnosic acid, resveratrol and silibinin.

Pre-infection priming with these non-pharmaceutical phytochemicals mitigate the risks of cardiac fibrosis following molecular attack by pathogens: quercetin, luteolin, hesperetin, baicalein, cyanidin, resveratrol, artemisinin, andrographolide, glycyrrhizin and tinospora cordifolia.

Pre-infection priming with these non-pharmaceutical compounds help mitigate the risks of myocarditis following molecular attack by pathogens: astragalus, Coenzyme Q10, vitamin C, ginger and thymoquinone.

Covid can induce renal fibrosis. Evidence from patient autopsy samples shows that Covid directly infects kidney cells and is associated with increased tubulo-interstitial fibrosis. Studies using human kidney organoids derived from pluripotent stem cells confirm that Covid infection leads to injury and dedifferentiation of renal cells, activation of profibrotic signaling pathways, and increased collagen 1 protein expression, indicating fibrotic changes. These effects occur independently of systemic immune responses, suggesting a direct role of the virus in driving fibrosis.

Pre-infection priming with these non-pharmaceutical compounds mitigate the risks of renal fibrosis following molecular attack by pathogens: dihydromyricetin, quercetin, curcumin, resveratrol, EGCG, berberine, salidroside, cordyceps, ginkgo biloba, ashwagandha and andrographolide.

Covid infection promotes endothelial-to-mesenchymal transition (EndMT), a process where endothelial cells lose their characteristic markers and acquire a fibroblast-like phenotype, contributing to vascular dysfunction and fibrosis. This transition is driven by the virus's interaction with endothelial cells, leading to the downregulation of ACE2 and subsequent upregulation of TGF-β1, a key cytokine that activates the TGF-β signaling pathway and induces EndMT. Oxidative stress and inflammation further amplify this process, with TGF-β enhancing redox imbalance and activating transcription factors like Smad and NF-κB, which promote fibrotic gene expression. Notably, different Covid proteins contribute to EndMT with varying potency: the Nucleocapsid (N) protein is more effective than the S protein, acting through a TLR4-ROS-TGF-β2-MRTF-A/B pathway, while the S protein primarily acts via ACE2 downregulation and TGF-β1-MRTF-B signaling. These changes result in endothelial barrier disruption, increased cell motility, and impaired capillary formation, which may underlie long-term complications such as pulmonary and cardiac fibrosis in post-Covid conditions. EndMT is considered a significant biological mechanism that increases cancer risk by shaping a tumor-supportive microenvironment and enabling metastatic spread.

Pre-infection priming with these non-pharmaceutical compounds mitigate the risks of EndMT following molecular attack by pathogens: moringa, resveratrol, honokiol and andrographolide.

Mitigation of existing cellular injuries and dysfunction caused by harmful epigenetic reprogramming is comprised of: (1) overcoming cellular exhaustion and cellular dysfunction of the various immune cell types which comprise CMI; (2) tight junction dysfunction; (3) glycocalyx restoration and maintenance; (4) mitochondrial dysfunction; (5) endothelial injuries and dysfunction and (6) dysbiosis.

Immune cell exhaustion and immune cell dysfunction can be induced by many types of viral infections. Chronic viral infections, such as those caused by human immunodeficiency virus (HIV), Covid, hepatitis B virus (HBV), and hepatitis C virus (HCV), lead to a state of T cell exhaustion characterized by a progressive loss of effector functions, including reduced cytokine production and cytotoxicity. This dysfunctional state arises due to persistent antigen exposure and is marked by the upregulation of co-inhibitory receptors such as PD-1, LAG-3, TIM-3, and CTLA-4.

Exhausted CD8+ T cells exhibit impaired proliferative capacity, metabolic dysregulation, and poor memory recall, which hinder effective viral clearance. While initially described in the context of chronic lymphocytic choriomeningitis virus (LCMV) infection in mice, similar exhaustion profiles have been observed in human chronic viral infections. Persistent overstimulation of T cells during these infections drives the exhaustion program, which represents a distinct state from other forms of dysfunction such as anergy or senescence.

Moreover, immune cell exhaustion is not limited to CD8+ T cells; CD4+ T cells also display signs of dysfunction during chronic viral infections, although their exhaustion profile differs from that of CD8+ T cells. The induction of exhaustion by chronic viruses represents a mechanism by which the immune system limits immunopathology, albeit at the cost of failing to eliminate the pathogen.

Pathogens can induce immune cell exhaustion and immune cell dysfunction in NK cells, dendritic cells, macrophages, neutrophils & monocytes. Dysfunctional chromatin inducibility leading to gene silencing, dysfunctional biological rheostats leading to chronic oxidative stress, chronic inflammatory states, dysregulated cellular injury protections, harmful metabolic reprogramming which impair metabolic switching and metabolic signaling networks, conditional deficiencies in microRNAs, chronic endothelial dysfunction, chronic mitochondrial dysfunction and chronic dysbiosis set the stage for immune cell exhaustion and dysfunction in all cells comprising CMI. Functional Immunological Fitness simultaneously mitigates all of these pathologies which underlie immune cell exhaustion and immune cell dysfunction.

Furthermore, the presence of dormant reservoirs of live Covid virus and persistent Covid spike protein also contribute to immune cell exhaustion and dysfunction.

Checkpoint inhibitors play a crucial role in overcoming immune cell exhaustion and dysfunction by blocking inhibitory signals that render various types of immune cells dysfunctional. For example, immune checkpoint inhibitors (ICIs) show significant potential in overcoming immune dysfunction in macrophages, dendritic cells (DCs), and monocytes, primarily by reversing their immunosuppressive phenotypes and restoring anti-tumor and anti-pathogen functions. Targeting checkpoint molecules like PD-1, PD-L1, CD47, and Tim-3 on these myeloid cells can shift their polarization from pro-tumor M2 or immunosuppressive states to inflammatory, anti-tumor M1 phenotypes, thereby enhancing phagocytosis, antigen presentation, and cytokine production. Checkpoint inhibitors also help overcome cellular exhaustion in NK cells.

Pre-infection priming with these non-pharmaceutical compounds helps to safely induce sustained checkpoint inhibition: berberine, curcumin, EGCG, gallic acid, ginseng, resveratrol, silibinin, hesperidin, luteolin, cyanidin, quercetin, baicalein, chlorogenic acid, lycopene and sulforaphane.

Beyond functional Immunological Fitness and sustained checkpoint inhibition achieved through non-pharmaceutical phytochemicals, pre-infection priming of NK cells, T cells, dendritic cells and macrophages can help overcome immune cell exhaustion and dysfunction.

Pre-infection priming with these non-pharmaceutical molecules prime NK cells: quercetin, vitamin A, vitamin C, vitamin B12, ginseng, curcumin, garlic, resveratrol, ashwagandha, andrographolide, astragalus, echinacea, myricetin, cyanidin, luteolin, R-α-lipoic acid, urolithin, sulforaphane, lactoferrin, ginkgo biloba, magnesium, selenium, inositol, zinc, IL15 (primed by vitamin D) and beta glucan.

Pre-infection priming with these non-pharmaceutical compounds prime macrophages: vitamin D, vitamin A, baicalein, squalene, omega-3 fatty acids, R-α-lipoic acid, butyrate, chlorogenic acid, cyanidin, gallic acid, ellagic acid, tannins, resveratrol, ursolic acid, sulforaphane, inositol, quercetin, amla, astaxanthin, fucoxanthin, beta glucan, lutein, lycopene and Coenzyme Q10.

Pre-infection priming with these non-pharmaceutical compounds prime T cells: hesperidin, naringenin, baicalein, EGCG and artemisinin.

Pre-infection priming with these non-pharmaceutical compounds help to overcome cellular dysfunction in dendritic cells: vitamin D, EGCG, curcumin, lycopene and quercetin.

Viral infections can impair tight junctions (TJ). Pathogenic viruses overcome the epithelial mucosal barrier by disrupting tight junctions, which opens paracellular space and facilitates viral penetration and spread. This disruption can occur through direct interaction of viral proteins with tight junction proteins or indirectly by activating proinflammatory cytokines and signaling pathways. For example, Covid binds to the protein associated with Lin seven 1 (PALS1) and zonula occludens-1 (ZO-1), disrupting tight and adherens junctions. ZO-1 is a central scaffolding protein in the cytoplasmic plaque of tight junctions and is fundamental to TJ structure and function. PALS1 is a membrane-associated guanylate kinase protein that plays a critical role in the formation and regulation of TJs in epithelial cells.

Pre-infection priming with these non-pharmaceutical compounds provide continuous cellular defenses against pathogen attacks on tight junctions: butyrate, propionate, acetate, forskolin, bifidobacteria probiotics, Lactobacillus rhamnosus postbiotic, lactobacillus paracasei postbiotic, glutamine, arginine, tryptophan, alpha keto glutarate, omega-3 fatty acids, vitamin A, vitamin C, vitamin K2, quercetin, berberine, naringenin, resveratrol, curcumin, EGCG, hesperidin, myricetin, xanthohumol, capsaicin, chlorogenic acid, glycyrrhizin, ginger, cinnamon, sulforaphane, taurine and human milk oligosaccharides.

The endothelial glycocalyx is a carbohydrate-rich layer composed of proteoglycans, glycosaminoglycans (GAGs), and plasma proteins that lines the luminal surface of endothelial cells in all blood vessels, from capillaries to large arteries and veins. It serves as a critical barrier and regulator, acting as a selective permeability filter that prevents large molecules like dextran and red blood cells from penetrating the endothelial surface while allowing the passage of smaller molecules such as plasma proteins. This layer plays a vital role in maintaining vascular integrity by modulating vascular tone through mechanotransduction of fluid shear stress, promoting nitric oxide production, and retaining anticoagulant factors like antithrombin III and complement regulators. Additionally, the glycocalyx protects endothelial cells from oxidative stress, cytokines, and circulating immune cells, and its integrity is essential for preventing leukocyte and platelet adhesion, thereby reducing inflammation and thrombosis.

The glycocalyx on human gut epithelial cells serves as a critical protective and functional layer. It acts as a physical barrier, preventing pathogenic bacteria and viruses from invading the intestinal tissue by forming a meshwork of carbohydrate moieties on glycoproteins and glycolipids, including transmembrane mucins like MUC1, MUC13, and MUC17. This barrier also provides lubrication, protects against ulcers and autodigestion, and functions as a size-selective diffusion barrier. The glycocalyx is essential for nutrient absorption and hosts commensal bacteria, providing binding sites that promote a healthy gut microbiota while limiting pathogen colonization. Furthermore, it plays a role in cellular signaling and is composed of proteoglycans, with heparan sulfate and hyaluronic acid being the predominant glycosaminoglycan components. Disruption of the glycocalyx is linked to gastrointestinal diseases such as inflammatory bowel disease and cancer.

Viruses can both degrade the host cell's glycocalyx and exploit it for infection and immune evasion. Some viruses, like H1N1, induce endothelial glycocalyx degradation through mechanisms involving matrix metalloproteinases (MMPs) and neuraminidase, contributing to vascular dysfunction and lung injury. Conversely, other viruses, particularly persistent ones like herpesviruses and hepatitis viruses, manipulate the host's glycan machinery to coat their virions and infected cells with sialic acid-rich glycans, forming a “viral glycocalyx” that helps them evade immune detection by engaging inhibitory Siglec receptors on immune cells. This strategy allows viruses to mimic host cell surfaces and avoid attack by NK cells and T cells.

Covid attacks the endothelial glycocalyx causing glycocalyx degradation through multiple mechanisms, including direct binding and the resulting inflammatory response. The virus can bind to heparan sulfate (HS), a component of the glycocalyx, which facilitates its attachment to ACE2 receptors on endothelial cells, leading to cellular entry and damage. This interaction suggests that a healthy, thick glycocalyx may act as a physical barrier against the virus, but once infection occurs, the glycocalyx is rapidly damaged.

Immunological Fitness helps to restore and maintain the glycocalyx. Moreover, the method of continuous cellular defenses provides added protections to the glycocalyx on a continuous basis.

Vitamin D restores and protects the glycocalyx (eGC), particularly by inhibiting key mechanisms of its degradation. Studies indicate that vitamin D can reduce levels of pro-inflammatory cytokines like TNF-α, which are known to degrade the glycocalyx, and inhibit heparanase expression, an enzyme linked to eGC breakdown in conditions like diabetes and sepsis.

Pre-infection priming with these non-pharmaceutical compounds provide continuous protection of the glycocalyx in blood vessels and the gut: omega-3 fatty acids, fucoidan and taurine. Human milk oligosaccharides play a particular role in protecting the glycocalyx of intestinal epithelial cells.

Viral infections consistently induce mitochondrial dysfunction as a key mechanism to promote viral replication, evade immune responses, and alter host cell metabolism. Viruses target mitochondria to disrupt cellular energy production, increase reactive oxygen species (ROS), impair antiviral signaling, and modulate cell death pathways.

Functional Immunological Fitness provides continuous protections against pathogen-induced mitochondrial injuries.

Pre-infection priming with these non-pharmaceutical molecules augment the protections provided by Immunological Fitness with robust, multi-faceted continuous cellular defenses against molecular attacks on mitochondria by pathogens: squalene, inositol, carnitine, R-α-lipoic acid, curcumin, Coenzyme Q10, EGCG, quercetin, baicalein, dihydromyricetin, resveratrol, punicalagin, spermidine, astaxanthin, astragalus, berberine, pyrroloquinoline quinone (PQQ), amla, urolithin, omega-3 fatty acids, lycopene, andrographolide, cyanidin, magnesium, taurine, proline, histidine, glycine, betaine, branched chain amino acids (leucine, isoleucine, and valine), glutamine, methionine, threonine, and lysine.

Viral infections can induce endothelial dysfunction through various mechanisms, including direct infection of endothelial cells, immune activation, cytokine release, and disruption of vascular integrity. Hemorrhagic fever viruses such as Ebola, Dengue, and Yellow Fever are known to infect endothelial cells and cause vascular dysfunction, leading to increased permeability, coagulopathy, and organ failure. For example, Dengue virus (DENV) infection can increase capillary permeability via a “cytokine tsunami” involving high levels of IL-2, IL-6, IL-8, TNF-α, and IFN-γ, and can also drive basement membrane degradation through MMPs.

Vitamin D deficiency is a risk factor for endothelial dysfunction (ED), which is characterized by reduced bioavailability of nitric oxide (NO), a key vasodilator, and is an early event in atherosclerosis development. Vitamin D exerts protective effects on the endothelium by regulating NO synthesis through the endothelial nitric oxide synthase (eNOS) pathway, counteracting the production of reactive oxygen species (ROS) by inhibiting NADPH oxidase, and enhancing antioxidant capacity. It also suppresses inflammation by inhibiting the NF-κB pathway and reducing the expression of proinflammatory mediators like TNF-α, IL-6, and adhesion molecules such as ICAM-1 and VCAM-1. Clinical studies have shown an inverse correlation between plasma 25(OH)D levels and endothelial function, with vitamin D deficiency linked to impaired endothelium-dependent vasodilation.

Functional Immunological Fitness mitigates many of the underlying cellular vulnerabilities which cause endothelial dysfunction.

Pre-infection priming with these non-pharmaceutical molecules augment the protections provided by Immunological Fitness with robust, multi-faceted continuous cellular defenses against molecular attacks on vascular endothelium by pathogens: glycine; proline, serine, cysteine, glutamine, arginine, histidine, lysine, methionine, phenylalanine, threonine, tryptophan, branched chain amino acids (leucine, isoleucine, and valine), tyrosine, citrulline, astragalus, vitamin B12, vitamin B6, quercetin, naringenin, baicalein, luteolin, grape seed extract. curcumin, resveratrol, ferulic acid, ashwagandha, omega-3 fatty acids and oleuropein.

Covid causes dysbiosis, which is an imbalance in the gut microbiota. Multiple studies have consistently demonstrated gut dysbiosis in patients with Covid, both during and after the illness. This dysbiosis is characterized by a reduction in beneficial bacteria such as Bifidobacterium, Faecalibacterium, Lactobacillus and Akkermansia and an increase in opportunistic pathogens like Enterococcus, Veillonella, and Clostridium species. Covid can directly contribute to dysbiosis through active replication in the gastrointestinal tract (GIT), even in the absence of gastrointestinal (GI) symptoms. The virus also infects intestinal cells via the ACE2 receptor, which is highly expressed in the gut lining, leading to cellular damage, inflammation, and disruption of gut barrier integrity. This damage enhances intestinal permeability, allowing bacterial translocation and triggering systemic inflammation. Additionally, the dysbiosis observed in Covid is linked to disease severity, with more pronounced microbial imbalances seen in severe cases. The altered gut microbiota affects immune responses by reducing the production of short-chain fatty acids (SCFAs) like butyrate, which are crucial for maintaining gut homeostasis, regulating immunity, and suppressing inflammation. Reduced SCFA levels impair tight junction formation, antimicrobial peptide secretion, and regulatory T cell function, further exacerbating immune dysregulation and inflammation. Dysbiosis may persist after recovery and is associated with post-acute sequelae of Covid (PASC or long Covid), contributing to chronic inflammation, gastrointestinal symptoms, fatigue, and cognitive impairment.

The method of Immunological Fitness and Continuous Cellular Defenses provides robust interventions on a daily basis to mitigate dysbiosis and restore a healthy gut microbiome utilizing the following non-pharmaceutical compounds: beta glucan, butyrate, human milk oligosaccharide, lactoferrin, fulvic acid, multiple prebiotics including fructooligosaccharide, partially hydrolyzed guar gum, durable spore type probiotics including Bacillus clausii and Bacillus coagulans and multiple heat killed postbiotics including akkermansia, Lactobacillus rhamnosus and Lactobacillus paracasei.Furthermore, three patented prebiotics are included which provide targeted restoration of injured beneficial bacteria. These are: galactooligosaccharides (Bimuno) which restores and maintains bifidobacterium, red potato (SolNul) which restores and maintains akkermansia and yellow kiwi (Livaux) which restores and maintains faecalibacterium prausnitzii.

Modern life comes with epigenetic hazards which people can not see or feel in the form of chronic oxidative stress and chronic inflammation which cause chronic cellular injuries such as chronic endothelial dysfunction and chronic impairments of mitochondria, DNA repair, tumor suppressor genes, autophagy and cell cycle abnormalities. Chronic oxidative stress and inflammation also induce conditionally essential nutrient deficiencies which further potentiate harmful epigenetic and metabolic reprogramming.

Because nearly 100% of modern people have dysfunctional CMI, they also have dysfunctional inducibility of chromatin, dysfunctional biological rheostats, dysfunctional metabolic signaling and switching, dysfunctional biological barriers in the form of dysfunctional tight junctions, dysfunctional glycocalyx and dysfunctional biological membranes all of which protect them against biological weapons.

These factors combine to make modern people vulnerable to harmful epigenetic and metabolic reprogramming which underlie the chronic disease epidemic, high vulnerability to epigenetic weapons and chronic gene silencing which leads to a vicious cycle of immune tolerance that may stay with them for the rest of their lives. This vicious cycle shortens lives by inducing cancers, heart attacks, strokes, neurodegenerative and autoimmune diseases. This vicious cycle directly contributes to autism, schizophrenia and depression and robs societies of workplace productivity and annual costs of hundreds of billions of dollars related excess healthcare costs, missed work and chronic physical impairments.

The method of epigenetic defense breaks the vicious cycle of harmful epigenetic and metabolic reprogramming faced by modern people by creating a continuous homeostasis, in the form of continuous cellular defenses against harmful epigenetic reprogramming. The continuous cellular defenses epigenetically rewires immune tolerance to trained innate immunity. This homeostasis restores chromatin inducibility, biological rheostats, biological barrier integrity and metabolic switching and signaling. It creates a continuous baseline of normalized oxidative stress and inflammation. Immune tolerance and trained innate immunity comprise two competing memory programs which are controlled by a metabolic switch in the form of nuclear factor kappa-light-chain-enhancer of activated B cells (NF-κB) under granulocyte-macrophage colony-stimulating factor (GM-CSF) control. NF-κB under GM-CSF control acts as an essential switch regulating the tolerization/training programs in hematopoietic stem and progenitor cells (HSPCs). Continuously saturated and functional vitamin D along with daily priming of non-pharmaceutical polyphenols such as EGCG, chlorogenic acid, curcumin, resveratrol, quercetin and ferulic acid continuously regulates NF-κB. In addition, the continuous optimization of all master regulators, but in particular the JAK/STAT Signaling and TNF alpha master regulators, achieved by functional Immunological Fitness and Continuous Cellular Defenses, maintain regulatory control over GM-CSF. In summary, the method continuously primes the process of epigenetic rewiring which transforms harmful epigenetic reprogramming to trained innate immunity. It does so by daily dosing of beta glucan and butyrate while continuously regulating GM-CSF and NF-κB. This homeostasis maintains the metabolic switch which protects and maintains trained innate immunity while inhibiting immune tolerance caused by harmful epigenetic reprogramming. Thus, population-scale biodefense against epigenetic weapons, now a requisite in the new age of synthetic biology if cities are to survive, creates the homeostasis for reverting harmful epigenetic memory to trained innate immunity, day in and day out to restore populations to robust CMI against epigenetic bioweapons and chronic diseases. Population-scale biodefense produces an inflection point of sustained human thriving, safety, and new economic power as never seen before.

Enhanced maintenance of master regulators beyond the baseline up-regulation provided by Immunological Fitness is comprised of continuous daily priming utilizing non-pharmaceutical compounds to optimize the function of each master regulator listed below:
NRF2 activators: andrographolide, sulforaphane, curcumin, resveratrol, carnosic acid, quercetin
Heme oxygenase-1 (HO-1) inducers: curcumin, resveratrol, chlorogenic acid, ginkgo biloba, carnosic acid, cyanidin, quercetin, EGCG, garlic, sulforaphane, thymoquinone
PPAR: echinacea purpurea, glycyrrhizin, honokiol, panax ginseng, cyanidin-3-glucoside,
AMPK: resveratrol, quercetin, EGCG, curcumin, berberine, ginseng, baicalein, dihydromyricetin
PI3/AKT/mTOR Signaling Pathway: curcumin, sulforaphane, quercetin, resveratrol, honokiol
Zonulin: butyrate, forskolin, taurine, glutamine, alpha keto glutarate, arginine, EGCG. berberine, resveratrol
IRAK: luteolin, curcumin, baicalein
MAPK/ERK Signaling: quercetin, silibinin, luteolin, EGCG, cyanidin, honokiol, berberine, artemisinin
HIF-1 alpha: garlic, thymoquinone, silibinin, berberine, fucoidan
SREBP Transcription Factors: sulforaphane, xanthohumol, berberine, tocotrienol
TNF alpha: curcumin, echinacea, EGCG, berberine, dihydromyricetin, naringenin, ginseng, carnitine, baicalein
JAK/STAT Signaling: resveratrol, curcumin, silibinin, ashwagandha, vitamin C, vitamin E, EGCG
Notch Signaling Pathway: curcumin, EGCG, resveratrol, quercetin, silibinin
FOXP3 proteins: vitamin A, resveratrol
SOCS Agonists: curcumin, luteolin, gallic acid, ginseng, ashwagandha, sulforaphane, EGCG, eugenol, ginger, echinacea, garlic, thymoquinone, quercetin

There is a small window of time at the very beginning of an illness when prevention of potentially lethal immune dysregulation and cytokine storm caused by epigenetic weapons is possible. This window begins as soon as a person becomes aware that they may be about to get sick with an infection. The symptoms are often mild. The initial symptoms may be a slight fever, a slight cough, a mild sore throat or a runny nose. This small window of time when symptoms are mild is the time to take action by consuming the first daily dose of the immediate treatment kit.

These early signs of possible infection suggest that a person has dysfunctional CMI. By taking the first dose of the immediate treatment kit as soon as possible and continuing to ingest a daily dose over the course of an additional nine days, it is possible to transform dysfunctional CMI to a state of marginally functional CMI to a degree that will activate enough first line innate immune defenses against infection to keep the antigen threshold below the key level which prevents the adaptive immune system from becoming activated. Immune dysregulation and cytokine storm only happen when an infection exceeds this key antigen threshold which caused the adaptive immune system to become activated. Functional CMI is usually capable of controlling and clearing infections before the level of infectious antigen rises above this key antigen threshold.

The immediate treatment kit “jump starts” numerous components of the innate immune system simultaneously. These components are: (1) rapid elevation of the serum vitamin D level to a level which approximates zero-order pharmacokinetics of vitamin D; (2) rapid normalization of vitamin B9 (methyl folate), vitamin B12, vitamin B6 and vitamin B2 which together help to normalize the levels of intracellular glutathione; (3) rapid replenishment of all critical micronutrients; (4) rapid replenishment of all conditionally essential amino acids and other non-amino acid nutrients which are classified as conditionally essential; (5) microRNAs; (6) rapid recovery of a healthful gut microbiome.

The contents of the immediate treatment kit include all ingredients included in the Continuous Cellular Defenses bioavailability optimized daily ingestible plus special innovations which are only included in the immediate treatment kit which rapidly activate CMI. These are: (1) specialized daily dosing of vitamin D which safely elevates the serum vitamin D level towards the level required to achieve zero-order pharmacokinetics without blood testing to measure serum vitamin D and calcium. The vitamin D dosing is taken only for ten days and is designed to be safe, even in people who may have elevated serum calcium but don't know it; (2) pulsed dosing of the probiotics bifidobacterium lactis and Lactobacillus rhamnosus totaling a dose of approximately 6 trillion organisms ingested over a ten day course . . . these live probiotics are separately packaged from other ingredients in the immediate treatment kit to preserve the viability of the high dose probiotics; (3) pulsed dosing of a heat killed lactobacillus paracasei postbiotic in a daily high dose equivalent to 50,000 tfu (total fluorescent units); (4) a delayed time-release capsule to provide a specialized mixture of antioxidant phytochemicals that will be released hours after the capsule is initially ingested and delivered into the body on a time-release basis; (5) pulse dosing of human milk oligosaccharide, lactoferrin and fulvic acid, each of which rapidly support gut barrier integrity and tight junctions.

Special characteristics are achieved by the immediate treatment kit as follows: (1) the pulsed daily dosing of two probiotics provide rapid replenishment of key beneficial bacteria which are known to be attacked by the Covid spike protein—the replenishment is provided in high concentrations of bacteria that are known to be safe but which are rarely used because of the relatively high cost of these ingredients; (2) rapid priming of plasmacytoid dendritic cells located in the intestinal tract with heat killed postbiotics—approximately 90% of type 1 interferon is produced by plasmacytoid dendritic cells in the gut-by circumventing the reliance on type 1 interferon priming of plasmacytoid dendritic cells by living cells in the gut—the method bypasses dependency on living gut bacteria which are often killed by the Covid spike protein. With heat killed postbiotics, the reliance of priming of type 1 interferon by living bacteria is bypassed—the method rapidly primes type 1 interferon production at the beginning stage of an infection when type 1 interferon production can make the difference between an infection remaining mild versus becoming severe; (3) the phytochemicals contained in the timed-release capsule have immunomodulatory and anti-inflammatory properties. They also mitigate oxidative stress. Included in the capsule is a specialized form of timed-release ginger granules. The timed-release capsule provides protection against the development of cytokine storm that is sustained for many hours beyond time the time of the initial ingestion of the immediate treatment kit each day; (4) higher doses of nattokinase and SAMe (S-Adenosyl-L-methionine)—SAMe is the bodies principal methyl donor and plays key roles in enzymatic reactions and in helping to rapidly normalize proper functioning of the TCA cycle. Higher dosing of nattokinase helps to safely degrade free floating Covid spike proteins as well as to mitigate the risk of thrombus formation; (5) pulse dosing of all conditionally essential amino acids (ie, glutamine), all critical micronutrients and phytochemicals which provide an array of microRNAs rapidly restore metabolic switching (which help to activate dysfunctional NK cells) and metabolic signaling which mitigates the risk of immune dysregulation and cytokine storm; (6) targeted restoration is an innovation that combines three patented prebiotics, each of which has proven efficacy in restoring key beneficial bacteria which are frequently depleted as a result of Covid. These are: galactooligosaccharides (Bimuno) which restores and maintains bifidobacterium, red potato (SolNul) which restores and maintains akkermansia and yellow kiwi (Livaux) which restores and maintains faecalibacterium prausnitzii.

Epigenetic weapons are the biological equivalent of a Trojan Horse. You can't feel or see the injuries which they cause until it's often too late to stop death or permanent, irreversible injuries. Epigenetic warfare is stealth warfare at population scale. Societies have no instincts about how to stay safe. Healthcare and government officials have no training or experience to guide those they lead on what they must do to stay safe. Tremendous confusion follows. Much of the official guidance is performative versus science based.

The only way to activate immunological fitness is by daily compliance by most people with a bioavailability optimized daily ingestible and a daily dose of vitamin D individualized to each person based upon initial and periodic blood testing. The consequences of non-compliance are tragic. Without compliance by most people on a daily basis, populations will remain vulnerable to mass death and disability. The infrastructure of cities and nations could collapse because there will not be enough people left to operate the complex infrastructures upon which cities rely: food distribution, energy, public safety, health care and more. Infrastructure collapse will be accompanied by economic collapse. Furthermore, without compliance there will be no possibility of achieving herd immunity. Without herd immunity, there will be no way to stop future pandemics. Also, the public indoor spaces such as buildings, buses and trains which cities require to function will become potential death traps and people will be afraid to enter. Furthermore, in democratic societies, people cannot be forced to comply. They can only be incentivized to comply.

How can they be motivated to protect themselves and others from danger which they can't see or measure? This method of national biodefense will not work without clear methods to help people visualize safety and danger. The insurance discounts which are integral to biodefense are based upon actuarial data as well as real time measurements of indoor respiratory viral pandemic risk. These data enable populations to see and measure biodefense safety and danger. Because compliance with biodefense is based upon activating Immunological Fitness and Continuous Cellular Defenses, the health of compliant people will dramatically improve in a measurable fashion which form the basis of insurance actuarial data. Thus, compliance with biodefense countermeasures qualifies both individuals and groups for special insurance discounts.

For individuals, the financial incentives go far beyond insurance savings. The cost of living will fall significantly, and people will feel noticeable improvements in their well-being. For organizations and businesses, workplace productivity will rise dramatically while absenteeism and workplace accidents will fall dramatically. Morale and employee retention will rise, and people will live longer.

Without compliance incentives which also help people to see danger and safety and to visualize hope, cities will never be safe, national and local economies will never be stable and the risk of mass death from stealth bioweapons will remain.

The method is built upon three unique non-pharmaceutical formulations. The first formulation is a bioavailability optimized daily ingestible powder which restores and maintains Immunological Fitness. The second formulation is a bioavailability optimized liquid daily ingestible and a bioavailability optimized powder daily ingestible. The liquid and powder components are blended at the time of use. The second formulation restores and maintains both Immunological Fitness and Continuous Cellular Defenses. The third formulation is an immediate treatment kit which is a modified version of the second formulation. It is designed to “jump start” CMI in people who may be in the early phase of an infection. It's purpose is to mitigate the risk of immune dysregulation and cytokine storm by rapidly activating at least partially functional CMI in order to control and clear an incipient infection before it exceeds the key antigen threshold which triggers the activation of adaptive immunity. The compositions, formulations, components, and pathways described herein may be physically packaged and distributed together as kits, including population-scale kits, emergency kits, preparedness kits, and maintenance kits. Such kits may comprise two or more ingestible compositions, optional devices or sensors, and printed, digital, or electronic instructions or guidance, and may be configured for individual, group, institutional, or population-scale deployment without departing from the scope of the disclosure. The kits and components described herein may further be deployed, provided, sold, or licensed as part of integrated systems, including systems comprising distribution mechanisms, data association components, compliance assessment components, and actuarial or insurance qualification components. The compositions and components described herein may be physically packaged together as kits, including with printed or electronic instructions, for distribution to individuals or populations. The system may be provided, sold, or licensed as a system.

The strategy behind the three formulations is designed to provide protection against mass death and disability caused by bioweapons to as many people as possible as quickly as possible. Because nearly 100% of the population presently has dysfunctional CMI, the scale of the present vulnerability to death by cytokine storm is extremely large. Meeting this need requires a formulation that contains ingredients which will be readily available in very large quantities. The first formulation meets this requirement.

It contains the following essential amino acids and conditionally essential nutrients: taurine, myoinositol, chiroinositol, N-acetyl glucosamine, N-acetyl cysteine, arginine alpha keto glutarate, L-glutamine, L carnitine fumarate, betaine anhydrous, citrulline DL malate, branched chain amino acids, lysine, phenylalanine, methionine, histidine, tryptophan, threonine, proline, creatine, serine, glycine and L-tyrosine.

It also contains the following essential micronutrients: magnesium, zinc, selenium, iron, trace minerals (including boron, copper and manganese), vitamin A, vitamin C, vitamin K2, omega-3 fatty acids, N-acetyl cysteine and R-α-lipoic acid. It contains the bioactive form of methyl folate which bypasses the methyl folate reductase enzyme which is frequently impaired by polymorphisms. It also contains a bioavailability optimized forms of vitamin B1, B2, B5 and B6. It contains high levels of the three forms of vitamin B12 (hydroxocobalamin, methylcobalamin and adenosylcobalamin) which is an innovation that enables people with polymorphisms affecting B12 metabolism to often overcome limitations caused by these B12 related polymorphisms.

It contains two forms of nicotinamide riboside (NR): NR alpha ketoglutarate and NR malate, both of which serve as NAD+boosters to make hijacking of cellular energy production more difficult for pathogens.

It contains a variety of prebiotics, spore forming probiotics and postbiotics.

It contains a mixture of polyphenols which provide a variety of microRNAs and act as antioxidants and anti-inflammatory agents. These include grape seed extract, resveratrol, berberine, curcumin, andrographolide, piperine, and quercetin. PQQ and Coenzyme Q10 are also included.

The second formulation includes everything in the first formulation but also includes specialized ingredients which will likely be supply chain limited. These include human milk oligosaccharide, lactoferrin, beta glucan, butyrate, xanthohumol, ergothioneine, astaxanthin, salidroside, fucoxanthin and tocotrienol. The second formulation will initially be prioritized for use by militaries and first responders.

The third formulation includes everything in the second formulation plus includes doses of vitamin D at approximately 25,000 IU's daily, high dose probiotics, specialized prebiotics to achieve targeted restoration of beneficial gut bacteria that are often killed by Covid spike proteins and high dose lactobacillus paracasei postbiotic to rapidly prime the production of type I IFN by plasmacytoid dendritic cells. The third formulation also includes a time release capsule with multiple polyphenols which act to mitigate the risk of cytokine storm over a longer period of time. This includes a timed release form of ginger.

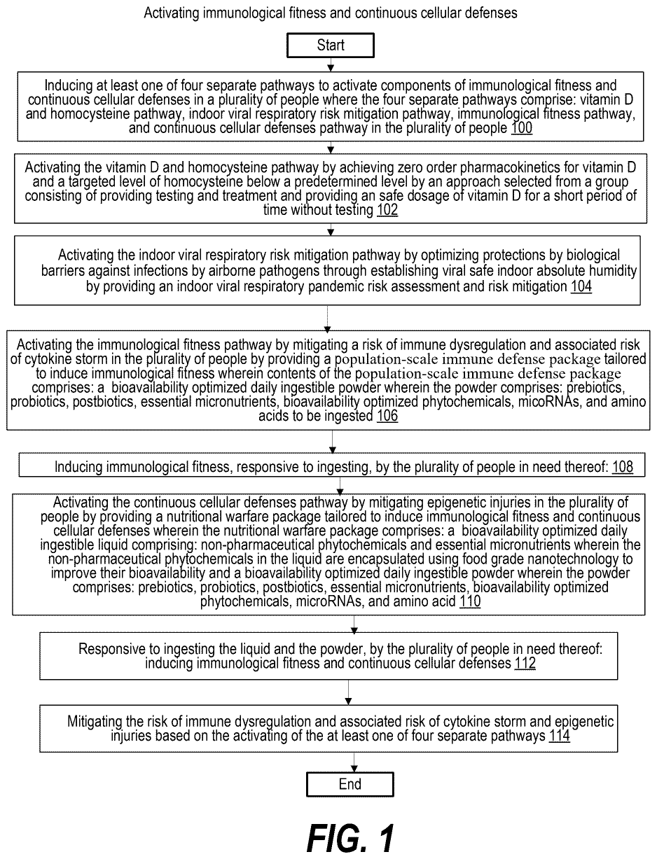

Turning now to FIG. 1, a flow chart for a process of activating immunological fitness and continuous cellular defenses is depicted in accordance with an illustrative embodiment. The process activates immunological fitness and continuous cellular defenses by inducing at least one of four separate pathways to activate components of immunological fitness and continuous cellular defenses in a plurality of people (step 100). The four separate pathways include a vitamin D and homocysteine pathway, an indoor viral respiratory risk mitigation pathway, an immunological fitness pathway, and a continuous cellular defenses pathway. The process activates the vitamin D and homocysteine pathway by achieving zero-order pharmacokinetics for vitamin D and a targeted level of homocysteine below a predetermined level by an approach selected from a group consisting of providing testing and treatment and providing a safe dosage of vitamin D for a short period of time without testing (step 102). Zero-order pharmacokinetics describes a process where a constant amount of a drug is eliminated per unit of time, independent of the drug's concentration in the body. This occurs when the body's elimination mechanisms, such as metabolic enzymes, become saturated and operate at their maximum capacity, meaning the rate of elimination does not increase even if the drug concentration rises. The process activates the indoor viral respiratory risk mitigation pathway by optimizing protections by biological barriers against infections by airborne pathogens through establishing viral safe indoor absolute humidity by implementing an indoor viral respiratory pandemic risk assessment and risk mitigation (step 104). The process activates the immunological fitness pathway by mitigating a risk of immune dysregulation and associated risk of cytokine storm in the plurality of people by providing a population-scale immune defense package tailored to induce immunological fitness wherein contents of the population-scale immune defense package comprises: a bioavailability optimized daily ingestible powder wherein the bioavailability optimized daily ingestible powder comprises: prebiotics, probiotics, postbiotics, essential micronutrients, bioavailability optimized phytochemicals, microRNAs, and amino acids to be ingested (step 106). The process induces immunological fitness in the plurality of people in need thereof responsive to ingesting contents of the population-scale immune defense package (step 108). The process activates the continuous cellular defenses pathway by mitigating epigenetic injuries in the plurality of people by providing a nutritional warfare package tailored to simultaneously induce both immunological fitness and continuous cellular defenses wherein the nutritional warfare package comprises: a bioavailability optimized daily ingestible liquid comprising: non-pharmaceutical phytochemicals and essential micronutrients wherein the non-pharmaceutical phytochemicals in the liquid are encapsulated using food grade nanotechnology to improve their bioavailability and a bioavailability optimized daily ingestible powder wherein the bioavailability optimized daily ingestible powder comprises: prebiotics, probiotics, postbiotics, essential micronutrients, bioavailability optimized phytochemicals, microRNAs, and amino acids (step 110). The process induces immunological fitness and continuous cellular defenses responsive to ingesting the bioavailability optimized daily ingestible liquid and the bioavailability optimized daily ingestible powder, by the plurality of people in need thereof (step 112). The process mitigates the risk of immune dysregulation and associated risk of cytokine storm and epigenetic injuries based on the activating of the at least one of four separate pathways (step 114). The process thereafter ends.

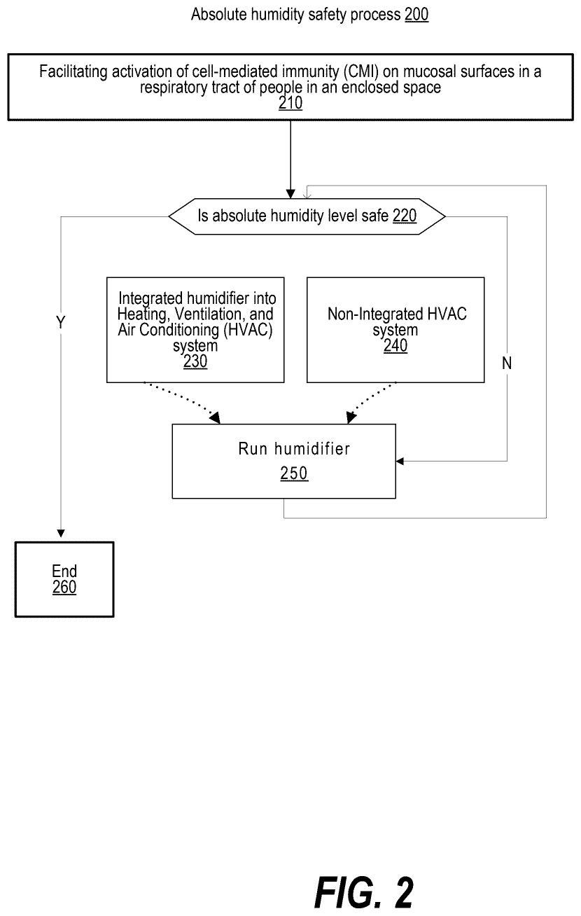

FIG. 2 shows the steps taken by an absolute humidity safety process 200. At step 210, the process facilitates activation of cell-mediated immunity (CMI) on mucosal surfaces in a respiratory tract of people in an enclosed space. The process determines as to whether the absolute humidity level is viral safe (decision 220). If the absolute humidity level is safe, then decision 220 branches to the ‘Y’ branch which proceeds to step 260 which ends the process for FIG. 2. On the other hand, if absolute humidity level is not safe, then decision 220 branches to the ‘N’ branch which proceeds to run humidifier at step 250 which loops back to decision 220. This looping continues until absolute humidity level is safe, at which point decision 220 branches to the ‘Y’ branch exiting the loop. The humidifier may be a fixed-installation humidifier or a portable humidifier. The type of humidifier may be, for example, but not limited to: A drum style (bypass) that uses a pipe to bring water directly to a reservoir (a pan) attached to the HVAC system. A disc wheel style (bypass) which is similar in design to the drum style humidifiers replacing foam drumming with a number of plastic discs with small grooves on both sides. A bypass flow-through style (bypass—also known as “biscuit style” or many other, similar variant names) uses a pipe to bring water directly to an electrically controlled valve at the top of the humidifier. Spray mist types use a pipe, usually a small plastic one, to bring water directly to an electrically controlled valve where an atomizer forces the water through a tiny orifice causing it to break up into tiny particles in the humidifier. Additional types may include non-bypass flow-through (fan augmented), steam, impeller or centrifugal atomizer, under duct designs, and the like. The humidifier may be either integrated into heating, ventilation, and air conditioning (HVAC) system 230 or not integrated in the HVAC system 240. In some embodiments, an absolute humidity humidifier may be built into the HVAC system and control of the humidifier may be built into the thermostat controlling the HVAC system.

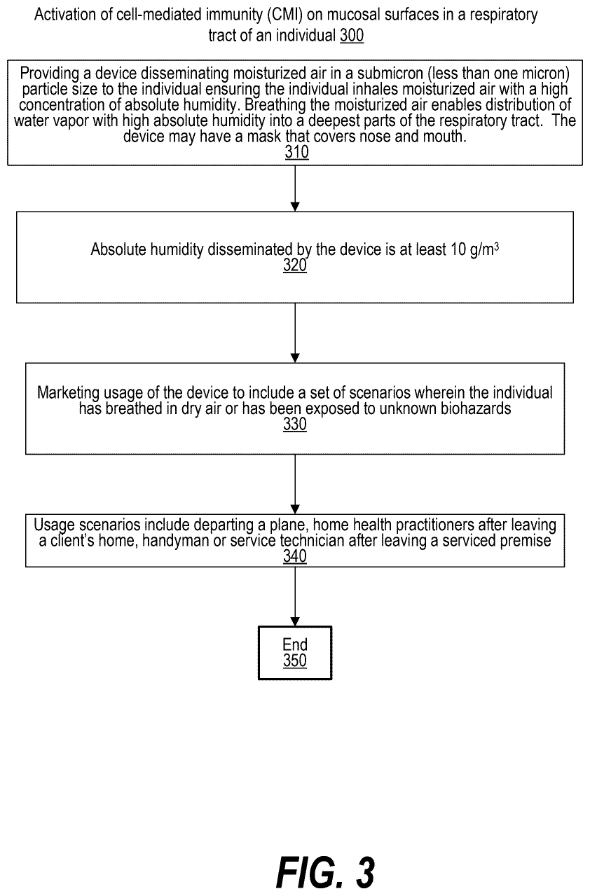

FIG. 3 shows the steps taken by a process that activates cell-mediated immunity (CMI) on mucosal surfaces in a respiratory tract of an individual 300. At step 310, the process provides a device disseminating moisturized air in a submicron (less than one micron) particle size to the individual ensuring the individual inhales moisturized air with a high concentration of absolute humidity level. Breathing the moisturized air enables distribution of water vapor with high absolute humidity into the deepest parts of the respiratory tract. The device may have a mask that covers nose and mouth. At step 320, the absolute humidity disseminated by the device is at least 10 g/m³. At step 330, the process may market usage of the device to include a set of scenarios wherein the individual has breathed in dry air or has been exposed to unknown biohazards. A portable device may be used to activate the CMI on the mucosal surfaces in the respiratory tract of the individual one or more times based on the situation. In some embodiments, the individual may be running an artificial intelligence (AI) mucosal services application which monitors the individual's environment and alerts the individual to use the portable device. At step 340, the usage scenarios for the process may include departing a plane, home health practitioners after leaving a client's home, repair person or service technician after leaving a serviced premise. FIG. 3 processing thereafter ends at 350.

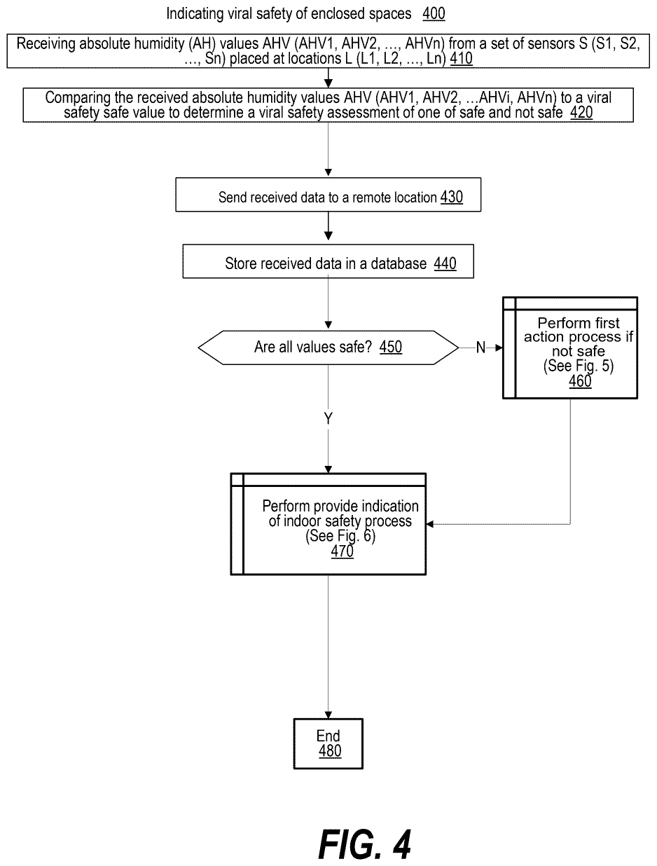

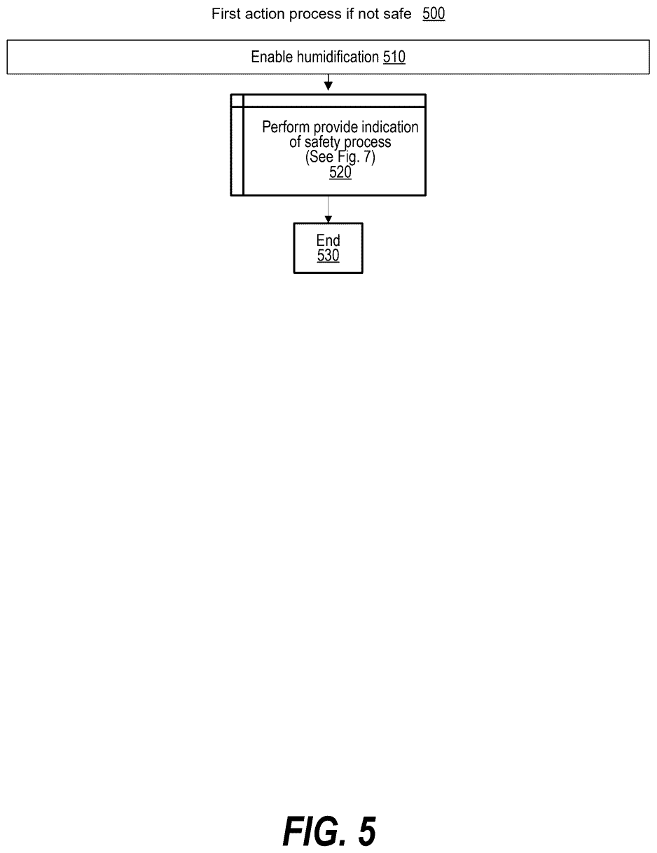

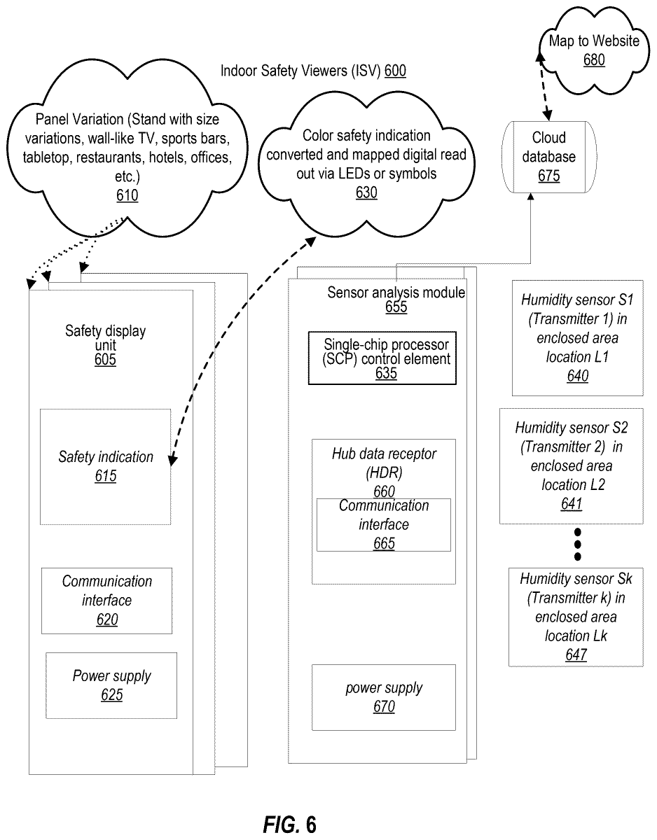

FIG. 4 shows the steps taken by a process that indicates viral safety of enclosed spaces 400. At step 410, the process receives absolute humidity (AH) values AHV (AHV1, AHV2, . . . , AHVn) from a set of sensors S (S1, S2, . . . , Sn) placed at locations L (L1, L2, . . . , Ln). At step 420, the process compares the received absolute humidity values AHV (AHV1, AHV2, . . . AHVi, AHVn) to a viral safety safe value to determine a viral safety assessment of one of safe and not safe. At step 430, the process sends received data to a remote location. At step 440, the process stores received data in a database. The process determines as to whether all values are safe (decision 450). If all values are safe, then decision 450 branches to the ‘Y’ branch. On the other hand, if not all values safe, then decision 450 branches to the ‘N’ branch. At predefined process 460, the process performs the first action process if not safe routine (see FIG. 5 and corresponding text for processing details). At predefined process 470, the process performs the provide indication of indoor safety process routine (see FIG. 6 and corresponding text for processing details). FIG. 4 processing thereafter ends at 480.

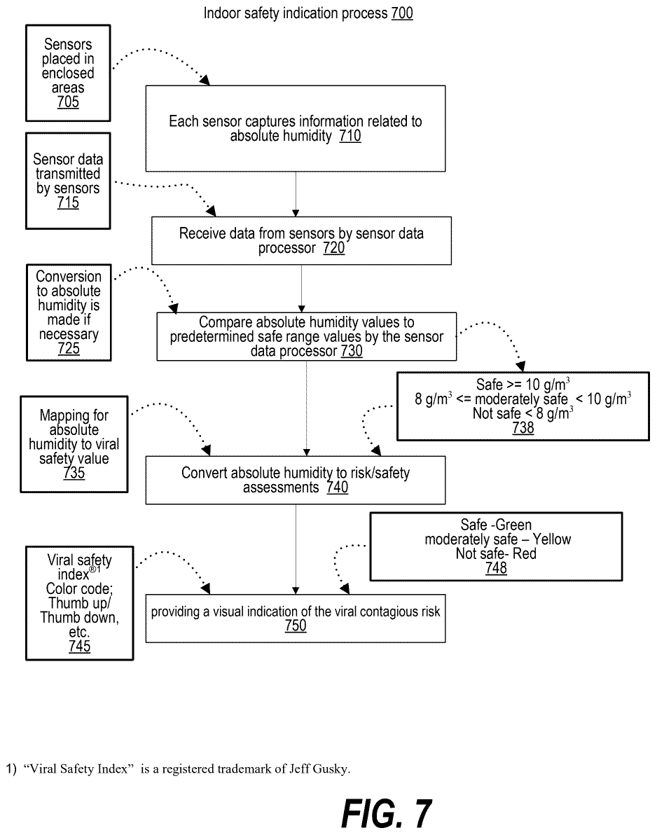

FIG. 5 processing shows the steps taken when the absolute humidity is not safe 500. At step 510, the process enables humidification. At predefined process 520, the process performs the provide indication of safety process routine (see FIG. 7 and corresponding text for processing details). FIG. 5 processing thereafter ends at 530.

FIG. 6 depicts a schematic view of an indoor safety viewer 600. In an embodiment, the indoor safety viewer comprises a means for a safety indication 615 derived from a plurality of humidity sensors S (S1, S2, . . . , Sk) placed at locations L (L1, L2, . . . , Lk) [640, 641, . . . , 647]. In an embodiment each humidity sensor Si in the plurality of sensors is placed in a corresponding enclosed location Li and each sensor Si is also transmitting humidity data. In an embodiment, the transmitted humidity data is received by hub data receptor 660 via communication interface 665 which is embedded in sensor analysis module 665 having its own power supply 670.

Although many sensor analysis modules may be used to support processing of many sensors, each sensor is configured to only be processed by one sensor analysis module. For illustration purposes, only safety display unit 605 is discussed. The sensor analysis module 655 may use single-chip processor (SCP) circuit control element 635 to analyze the received humidity data and to perform actions based on policies. The actions may include sending the data to cloud database 675 which supports map to website processing 680. The actions may include utilizing communication interface 665 to send information to a plurality of safety display units. Although the information being sent may be different for different safety display units and each unit may have distinctive characteristics, for illustration purposes, only safety display unit 605 is discussed. Safety display unit 605 receives information via communication interface 620, is powered via power supply 625, and provides safety indication 615. Safety display unit 605 may take the form of a panel with many implementation variations, for example, but not limited to a stand with size variations, a wall-like TV such as found in a home or sports bar, a tabletop such as found in restaurants, hotels, offices, and the like 610. The safety display unit 605 may have an adjustable size allowing for expansion or contraction. The display unit 605 may support a viral safety alert feature and provide audio warnings. The integrated system may provide a safety alert when weather conditions suggest low absolute humidity will be dangerous indoors in the near future. The safety indication 615 may be, for example, but not limited to a color safety indication based on a converted and mapped digital read out display via light-emitting diodes (LEDs). At step 630, the color safety indication may be converted and mapped as a digital read out via LEDs or as symbols, such as, a thumbs up or thumbs down indicator. Although it is possible that the plurality of humidity sensors captures and transmits information from which absolute humidity is derived, in an exemplary embodiment, each sensor in the plurality of sensors directly outputs absolute humidity. The values received by the hub data receptor 660 may be cached and compressed by the sensor analysis module 655 taking advantage of consecutive duplicate values reported by a single sensor Si representing changes of values at times or changes in times. Similar compression techniques may be used when there is insignificant variation in absolute humidity values between the single sensor Si and another single sensor Sj. The compressed data may be stored locally and/or sent to cloud database 675.

In an embodiment, the indoor safety apparatus 600 may have safety display unit 605, which may include safety indication 615, a communication interface 620, and a power supply 625. The safety display unit 605 may have various panel variations 610, such as, for example, but not limited to a stand with size variations, a wall-like TV such as in a sports bar, a tabletop configured to display information, such as, in a restaurant. The display may include LEDs lights. Humidity sensors transmitters 1, 2, . . . , k 640, 641, ..., 647 located in enclosed areas 1, 2, . . . , k capture humidity information and transmit the information to hub data receptor 660 in a sensor analysis module 655 that receives the transmitted information via a communication interface 665 utilizing a power supply 670. The power supply may be, for example, a battery, a USB connection, an alternative current (AC) power source, or even a solar powered power source or power source backup. The received humidity sensor data may be an absolute humidity value originating from the humidity sensors or may be converted to an absolute humidity value utilizing an algorithm loaded into the processor circuit control element 635. The sensor analysis module 655 converts the absolute humidity value to a form suitable for transmitting to the safety display unit 605. In addition, the sensor analysis module may also send the collected data to a cloud database 675. In some embodiments, data from the cloud database 675 may be sent or mapped for access at a website 680 corresponding to where the humidity sensor data is collected. The data may be collected, for example, from multiple different stores in a large building with separate offices or separate businesses. In an example embodiment, an entire mall may have a set of indoor safety viewer ISV (ISV₁, ISV₂, . . . , ISVₖ, ISVn), where each ISVₖ K=1,n collects humidity sensor data from a limited number of sensors that are close and may include multiple stores. A database may include an identification of placement for each sensor and a mapping of the placement to a store. In an embodiment, the real-time data including sensor measurements may be sent to the cloud database 675 and consolidated in a compressed form. The compressed form may, for example, identify a measurement, an initial start time of the measurement, and how long that measurement stayed the same. When the measurement changes, a change indication of when the change took place, and the change delta from the previous measurement. The cloud database 675 may support an application programming interface (API) allowing support from a web site or a service. The service may provide proactive notifications and/or alarms when indoor safety levels are not in the safe range. Support may be provided similar to a security system or integrated into a security system.

FIG. 7 shows the steps taken by an indoor safety indication process 700. At step 705, a plurality of sensors is placed at a plurality of locations, each location representing an enclosed area. At step 710, each sensor in the plurality of sensors captures information related to absolute humidity. At step 715, the sensor data is transmitted by sensors. At step 720, a sensor data processor receives sensor data. At step 725, conversions of the received data to absolute humidity are made if necessary. At step 730, the process compares absolute humidity values to predetermined safe range values. At step 738, the absolute humidity is characterized as safe when greater than or equal to 10 g/m³, which is the mapping for absolute humidity to viral safety value 735. The absolute humidity is characterized as moderately safe when the absolute humidity is less than 10 g/m³ and greater than or equal to 8 g/m³. The absolute humidity is characterized as unsafe when the absolute humidity is less than 8 g/m³. At step 740, the absolute humidity to risk/safety assessments are made based on the mappings in step 738. At step 745, the Viral Safety Index®² is depicted in some form, for example, via a color code, a Thumb up/Thumb down, etc. At step 750, a visual indication of the viral contagious risk is provided. At step 748, green is used to indicate safe, yellow is used to indicate moderately safe, and red is used to indicate not safe. ² VIRAL SAFETY INDEX is a registered trademark of Jeff Gusky.

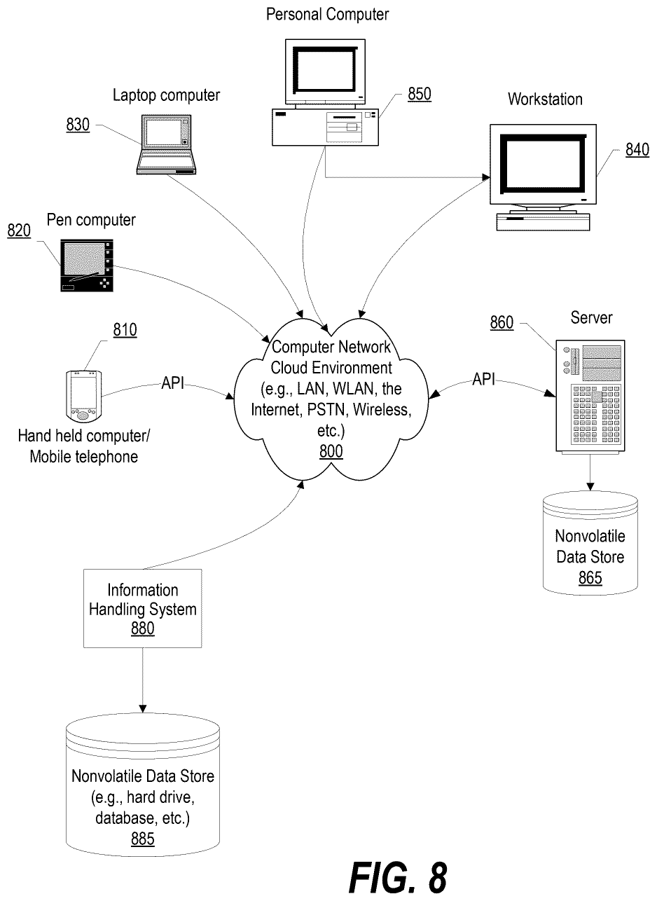

As shown, cloud computing environment 800 comprises one or more cloud computing nodes with which local computing devices used by cloud consumers, such as, for example, personal digital assistant (PDA) or cellular telephone 810, desktop computer 850, laptop computer 830, and/or other mobile device such as an automobile computer system may communicate by sending and receiving data as needed. Nodes in the computer network 800 may communicate with one another. They may be grouped (not shown) physically or virtually, in one or more networks, such as Private, Community, Public, or Hybrid clouds as described hereinabove, or a combination thereof. This allows cloud computing environment 800 to offer infrastructure, platforms and/or software as services for which a cloud consumer does not need to maintain resources on a local computing device. It is understood that the types of computing devices shown in FIG. 8 are intended to be illustrative only and that computing nodes in cloud computing environment 800 can communicate with any type of computerized device over any type of network and/or network addressable connection (e.g., using a web browser). Types of computer networks that can be used to interconnect the various information handling systems include Local Area Networks (LANs), Wireless Local Area Networks (WLANs), the Internet, the Public Switched telephone Network (PSTN), and others. Examples of handheld computer 810 include personal digital assistants (PDAs), personal entertainment devices, such as MP3 players, portable televisions, and compact disc players. Other examples of information handling systems include pen, or tablet, computer 820, laptop, or notebook, computer 830, workstation 840, personal computer system 850, and server 860 archival storage systems 865, etc. Other types of information handling systems that are not individually shown in FIG. 8 are represented by information handling system 880 shown with nonvolatile data store 885. As shown, the various information handling systems can be networked together using computer network 800. In an embodiment, mobile phone 810 may have an integrated absolute humidity sensor and transmit captured absolute humidity information as well as location information to server 860 utilizing an API supported by server 860. Other computing systems may also include similar absolute humidity processing support.

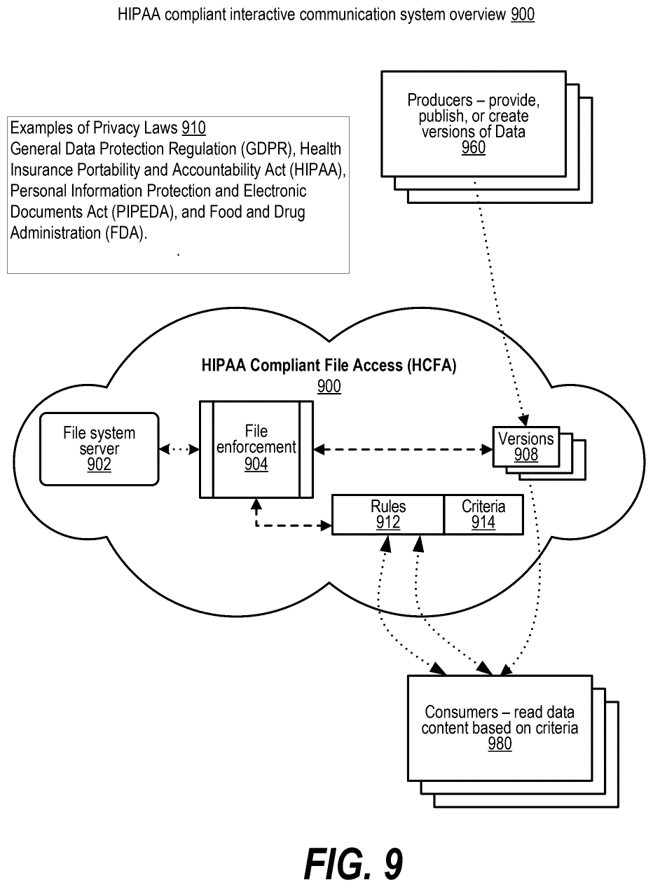

FIG. 9 depicts a schematic view of a HIPAA compliant interactive communication system overview 900. There are many existing legal file frameworks that are currently defined and approved or planned to be to be approved. Examples of privacy laws 910 include but are not limited to General Data Protection Regulation (GDPR), Health Insurance Portability and Accountability Act (HIPAA), Personal Information Protection and Electronic Documents Act (PIPEDA), and Food and Drug Administration (FDA).

Any of these file systems may be used to implement support for the capabilities described herein. When an updated version of data is added to the system, producers provide, publish, or create versions 908 of data 960. In an embodiment, a producer sends a request to add new data to a file system server 902. When the server 902 receives the request, the server 902 is responsible for ensuring data is encrypted as appropriate by utilizing a key manager 916. Proper usage of keys is important for separating access to data. There are many approaches for creating keys to be used to encrypt and decrypt data. In some embodiments, the strength of the keys and the complexity of preventing access to the keys may be chosen based on the sensitivity of the data. In some embodiments, the contents of the file are scanned for personal identity (PII) to determine the sensitivity of the data in the file. In some embodiments, the maximum classification of sensitivity found in the file may be used for the entire file, for example, a social security number in the file may be assessed as very sensitive. Different embodiments may use different rules, for example, there may be different levels of encryption based on a sensitivity of portions of the file, such as, by a mapping of field types to a level of sensitivity. As an example, the type of field may be known by a template of a document used to create the file. Then using information about the sensitivity of the data, the file system server 902 may ensure process file metadata enforcement 904 is configured to properly process data by setting up rules 912 and criteria 914 which allow consumers 980 to read data content based on and the rules 912 and the criteria 914. Although the privacy support is needed for user communications, absolute humidity information captured over time may also be processed based on consent.

FIG. 10 depicts a schematic diagram of a system having support for a sensor directory 1000. The sensor metadata 1010 is included as files on the file system. Each user may have user owned data with user files and defaults. Although various formats may be used, in one embodiment, each type of data is represented by a structure with a type of structure identified as a first field in the structure which be used as a case or switch statement for processing the data in the structure. Separate structure types may be used for representing various data, such as, for example, but not limited to sensor identification, access rules, location of sensor, owner of sensor, and the like. The system may support tracking information by owner of sensors. In another embodiment, sensor data may be written as records in a database management system (DBMS) which supports indexing, look up, and complex queries. Each sensor is assigned to only one hub data receptor (HDR) 660. In FIG. 10, HDR 1 1030 has HDR sensors 1 1035 having identifiers 1040 and access information 1045, HDR 2 1050 has HDR sensors 2 1055 having identifiers 1060 and access information 1065, . . . , HDR n 1070 has HDR sensors n 1075 having identifiers 1080 and access information 1085. Data read or written to each file are brought into cache lines (CL) and processed according to metadata and operations applied 1090. File access 1095 brings in file records (user data) into local cache line(s) to process the data. Although FIG. 10 depicts multiple HDRs with separated support, a system could be tailored to a single HDR tailored for a single user or a single owner with only one set of defaults for the single owner.

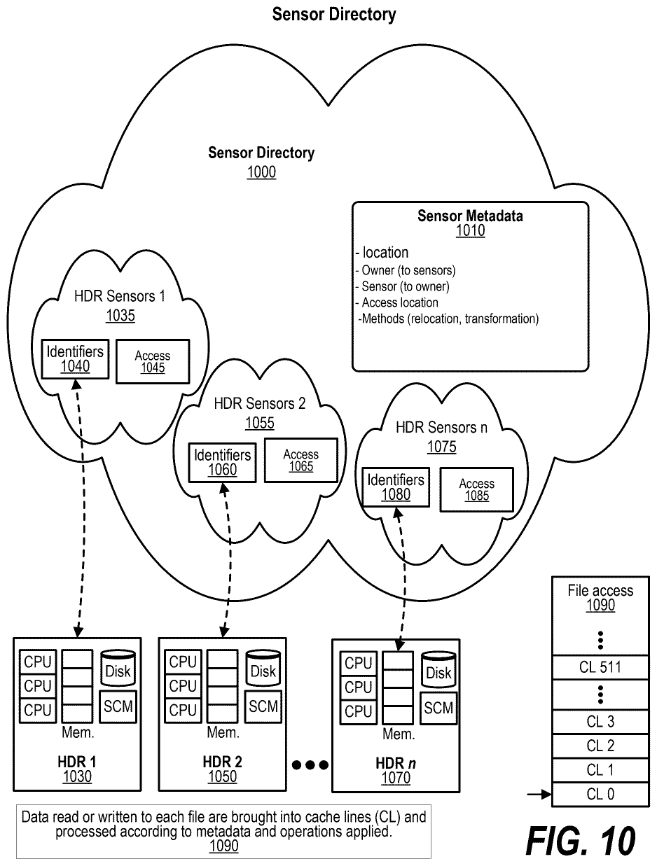

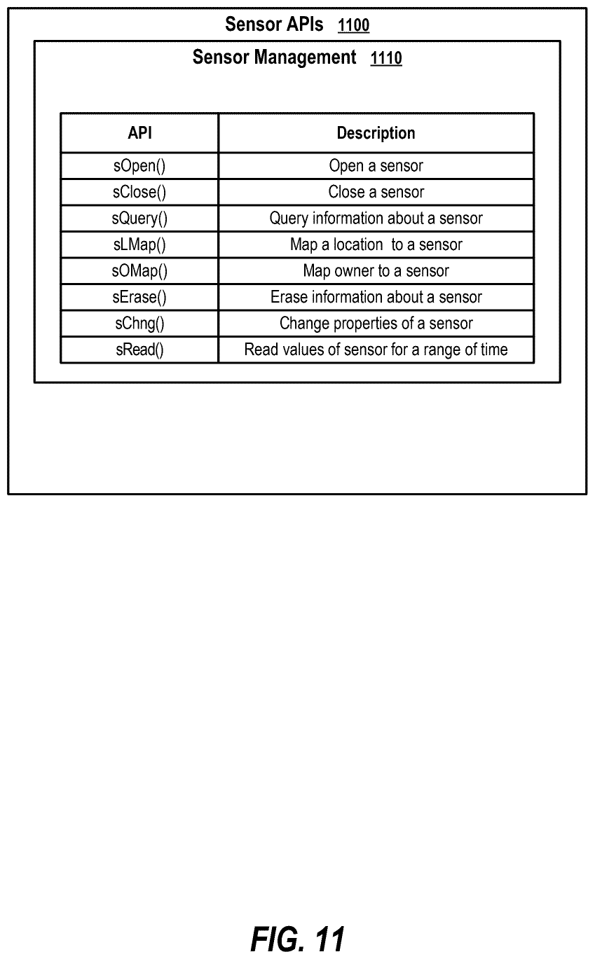

A list of example sensor APIs 1100 is included in FIG. 11. Examples of sensor management APIs 1110 include: sOpen() Open a sensor; sClose() Close a sensor; sQuery() Query information about a sensor; sLMap() Map a location to a sensor; sOMap() Map an owner to a sensor; sErase() Erase information about a sensor; sChng() Change properties of a sensor; sRead() Read values of sensor. Many other sensor APIs may be supported such as assigning a sensor to an HDR.

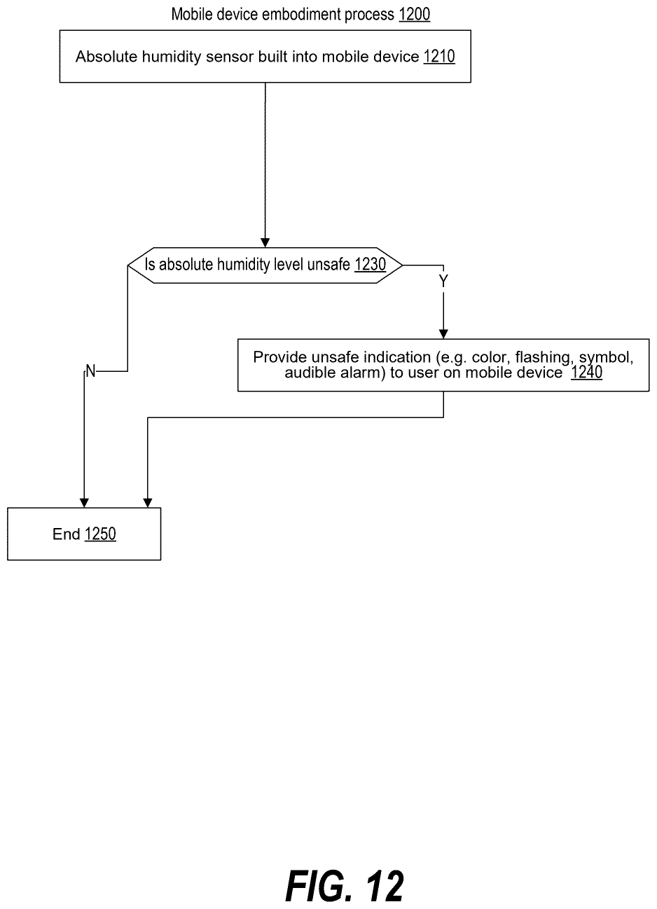

FIG. 12 shows an embodiment of an unsafe indication on mobile device process 1200. In an embodiment, the humidity sensor is built into mobile device 1210. The process determines as to whether the absolute humidity level is unsafe (decision 1230). If the absolute humidity level is unsafe, then decision 1230 branches to the ‘Y’ branch. On the other hand, if absolute humidity level is safe, then decision 1230 branches to the ‘N’ branch. At step 1240, the process provides unsafe indication to user on mobile device. The unsafe indication may be for example, a color, a flashing symbol, an audible alarm, or the like. FIG. 12 processing thereafter ends at 1250.

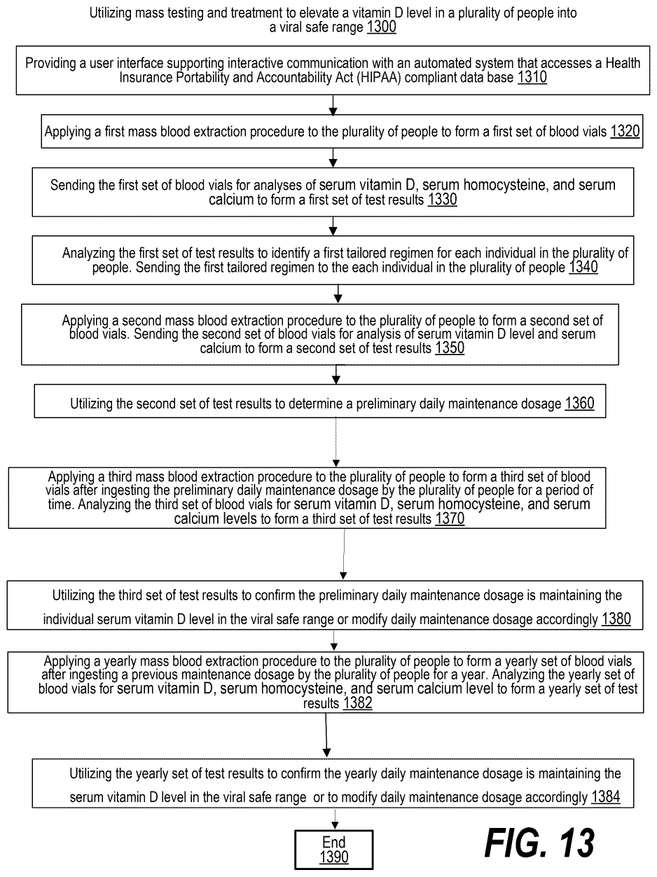

FIG. 13 shows the steps for a process that utilizes mass testing and treatment to elevate a vitamin D level in a group of people into a viral safe range 1300. At step 1310, the process provides a user interface supporting interactive communication with an automated system that accesses a Health Insurance Portability and Accountability Act (HIPAA) compliant database such as shown in FIG. 9. At step 1320, the process applies a first mass blood extraction procedure to the group of people to form a first set of blood vials. At step 1330, the process sends the first set of blood vials for analysis of calcium, magnesium, and serum vitamin D to form a first set of test results. At step 1340, the process analyzes the first set of test results to identify a first tailored regimen for each individual in the group of people. During the analysis of the first set of test results, the process identifies individuals with problematic blood levels of calcium or magnesium that may need medical intervention. Those identified individuals may receive personalized communications with a medical professional instead of automated communications by the user interface. Typically, those identified individuals will need to have the problematic blood levels addressed before being allowed to participate in the achieving safe level of vitamin D protocol. Typically, the first tailored regimen is determined based on an individual's current serum vitamin D level and body mass index (BMI); however, specialized algorithms may be used based on numerous factors, such as, other conditions such as any previous relevant illness such as sarcoidosis or parathyroid disease, chronic kidney disease, and the like. The requested information would include demography, age, height, weight, sex so body mass index (BMI) could be calculated. In an embodiment, the upper end of the normal range of serum vitamin D starts at least 55 ng/ml. In some embodiments the ramp up dosage is targeted to reach between 80 ng/ml and 100 ng/ml attempting to compensate for the current impairment of CMI.

The user interface may include logic to process these conditions automatically as part of the AI training. Also, feedback from tracked results may be used to improve accuracy of doses needed for ramp up and for maintenance. At step 1350, the process applies a second mass blood extraction procedure to the group of people to form a second set of blood vials. The second set of blood vials are sent for analysis of serum vitamin D level and calcium to form a second set of test results. At step 1360, the process utilizes the second set of test results to determine a preliminary daily maintenance dosage. At step 1370, the process applies a third mass blood extraction procedure to the group of people to form a third set of blood vials after ingesting the preliminary daily maintenance dosage by the group of people for a period of time. The third set of blood vials are sent for analysis of serum vitamin D level to form a third set of test results. At step 1380, the process utilizes the third set of test results to confirm the preliminary daily maintenance dosage is maintaining the serum vitamin D level in the viral safe range or to modify daily maintenance dosage. In an embodiment, the treatment pack is to be consumed daily for 20 days and the preliminary daily maintenance dosage is consumed daily for at least 2 months following completion of the 20 day pack. At step 1382, the process applies a yearly mass blood extraction procedure to the group of people to form a yearly set of blood vials after ingesting a previous maintenance dosage by the group of people for a year. Analyzing the yearly set of blood vials for serum vitamin D level to form a yearly set of test results. At step 1384, the process utilizes the yearly set of test results to confirm the currently daily maintenance dosage is maintaining an individual's serum vitamin D level in the viral safe range or to modify daily maintenance dosage as appropriate. FIG. 13 processing thereafter ends at 1390.

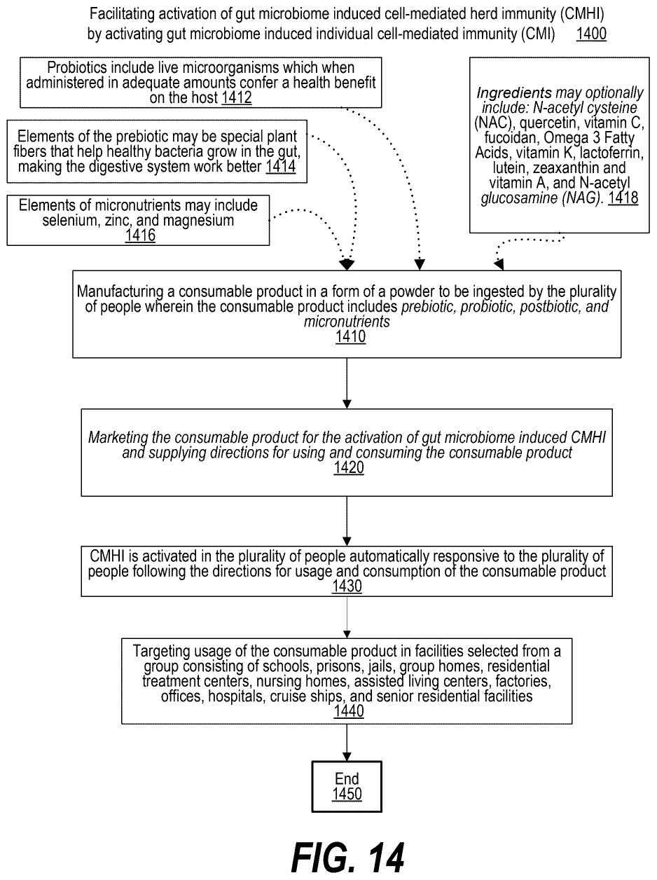

FIG. 14 shows the steps taken by a process that facilitates activation of gut microbiome induced CMHI by activating gut microbiome induced individual cell-mediated immunity (CMI) 1400. The gut microbiome powder is designed to correct for widespread dysbiosis (unhealthy gut microbiome) as well as widespread insufficiency of essential micronutrients which are critical to healthy immune function. Additional constituents, such as, but not limited to N-acetyl cysteine (NAC), quercetin, vitamin C, fucoidan, and N-acetyl glucosamine (NAG) 1418 may be part of the bioavailability optimized daily ingestible powder.

At step 1410, a consumable product is manufactured in a form of a powder to be ingested by a group of people wherein the consumable product includes prebiotic, probiotic, and micronutrients. Probiotics 1412 are live microorganisms that are intended to have health benefits when consumed or applied to the body. Prebiotics 1414 are special plant fibers that help healthy bacteria grow in the gut, making the digestive system work better. The micronutrients 1416 may include selenium, zinc, and magnesium. The ingredients 1418 may optionally include: N-acetyl cysteine (NAC), quercetin, vitamin C, fucoidan, and N-acetyl glucosamine (NAG). Although people often think of bacteria and other microorganisms as harmful “germs,” many are actually helpful. Some bacteria in the gut play a critical role in the body's immune system. Many of the microorganisms in probiotic products are the same as or similar to microorganisms that naturally live in the human intestinal tract.

At step 1420, the process markets the consumable product as facilitating the activation of gut microbiome induced CMHI and supplies directions for using and consuming the consumable product. At step 1430, CMHI is activated in the plurality of people automatically responsive to the plurality of people following the directions for usage and consumption of the consumable product. At step 1440, the process targets the usage of the consumable product by facilities selected from a group consisting of schools, prisons, jails, group homes, residential treatment centers, nursing homes, assisted living centers, factories, offices, hospitals, cruise ships, and senior residential facilities. FIG. 14 processing thereafter ends at 1450.

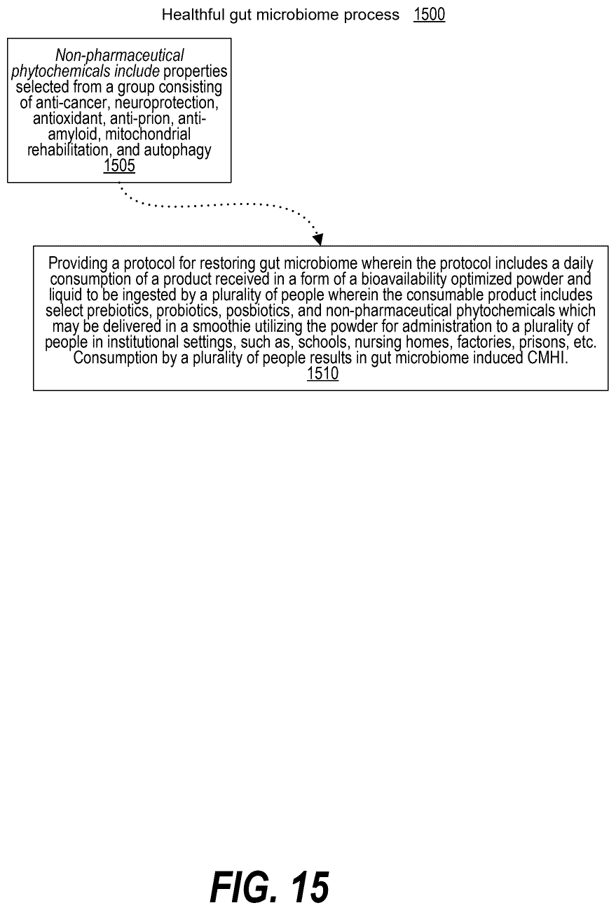

FIG. 15 shows the steps for restoring and maintaining a healthful gut microbiome 1500. At step 1510, the process provides a protocol for restoring gut microbiome. The protocol includes a daily consumption of a consumable product received in a form of a powder to be ingested by a group of people. The consumable product includes select prebiotics, probiotics, and non-pharmaceutical phytochemicals which may be delivered in a smoothie utilizing the powder for administration to a group of people in institutional settings, such as, schools, nursing homes, factories, prisons, etc. Consumption by the group of people results in gut microbiome induced CMHI. The non-pharmaceutical phytochemicals include properties selected from a group consisting of anti-cancer, neuroprotection, antioxidant, anti-prion, anti-amyloid, mitochondrial rehabilitation, and autophagy 1505. The consumable product may include N-acetyl cysteine (NAC), N-acetyl glucosamine (NAG), prebiotic, probiotic, and micronutrients. The consumable product may be included in a smoothie. Consumption of the smoothie by a group of people results in gut microbiome induced CMHI.

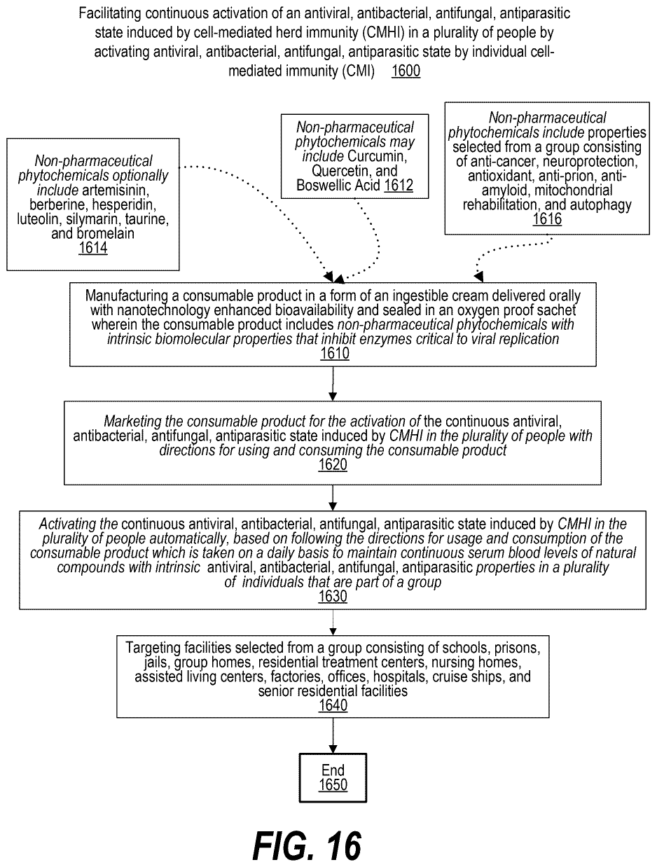

FIG. 16 shows the steps taken by a process that facilitates activation of antiviral priming induced CMHI by activating antiviral induced individual cell-mediated immunity (CMI) for long term usage 1600. At step 1610, the process provides a consumable daily pack to a plurality of people. The consumable daily pack includes contents that inhibits enzymes that promote viral replication to achieve anti-viral induced CMHI. The Antiviral Priming Daily Supplement is intended to be taken daily and facilitates Antiviral Priming CMHI. Like the Viral Fire Extinguisher®³, this product is designed to overcome the problems of bioavailability by using a nanotechnology delivery system to facilitate bioabsorption of otherwise poorly absorbed phytochemicals taken orally. This product emulates what has been seen in African countries with the lowest rates of Covid in the world. A significant percentage of the population in these countries have background levels of anti-parasite and antimalarial medications in their ³ Viral Fire Extinguisher Is a Registered Trademark of Jeff Gusky. bloodstreams at all times. These medications also have enzyme blocking characteristics that inhibit viral replication. The Antiviral Priming Daily Supplement helps mitigate the ever-present risk of new bioweapons, the increasing incidence of ADHD in young populations, cancer, neurodegenerative diseases, heart disease, strokes, autoimmune diseases caused by widespread spike protein endothelial disease and widespread impairment of cell-mediated immunity. At step 1620, the process manufactures a consumable product in a form of an ingestible cream delivered orally with nanotechnology enhanced bioavailability and sealed in an oxygen proof sachet wherein the consumable product includes non-pharmaceutical phytochemicals with intrinsic biomolecular properties that inhibit enzymes critical to viral replication. The non-pharmaceuticals and phytochemicals may include curcumin, quercetin, and boswellic acid 1612. The non-pharmaceuticals and phytochemicals optionally include artemisinin, berberine, hesperidin, luteolin, silymarin, taurine, and bromelain 1614. The non-pharmaceuticals and phytochemicals include properties selected from a group consisting of anti-cancer, neuroprotection, antioxidant, anti-prion, anti-amyloid, mitochondrial rehabilitation, anti-spike protein, omega-3 fatty acids, and autophagy 1616. At step 1630, the process markets the consumable product for the activation of the antiviral priming induced CMHI in the group of people with directions for using and consuming the consumable product. In an embodiment, the marketing targets facilities for example, but limited to schools, prisons, jails, group homes, residential treatment centers, nursing homes, assisted living centers, factories, offices, hospitals, cruise ships, senior residential facilities, and the like. At step 1640, the process activates the continuous antiviral state induced by CMI in the plurality of people automatically, based on following the directions for usage and consumption of the consumable product. FIG. 16 processing thereafter ends at 1660.

In an embodiment, the daily consumable product may include one or more of the following ingredients: squalene, homoharringtonine, vitamin K2 MK-7, fucoxanthin, astaxanthin, ahiflower oil, Coenzyme Q10, glutathione, R-alpha lipoic acid, N-acetyl cysteine, L-ergothioneine, ursolic acid, pterostilbene, resveratrol, naringenin, epigallocatechin gallate, dihydromyricetin, silibinin, lactoferrin, NAD+, PQQ, boswellic acid, glycyrrhizic acid, curcumin, lutein/zeaxanthin, quercetin, apple extract, grapeseed extract, berberine, silybin, olive extract, ginseng, andrographis, astragalus, fucoidan, beta glucan, butyrate, nattokinase, urolithin, malic acid, carnitine fumarate, thymoquinone, artemisinin, spermidine, moringa, taurine, holy basil, fulvic acid, L-glycine, galactooligosaccharide prebiotic, gold kiwi powder prebiotic, resistant potato starch prebiotic, Bacillus coagulans probiotic, Alkalihalobacillus, clausii probiotic, Lactobacillus rhamnosus postbiotic, n acetyl glucosamine, vitamin A, vitamin B12, vitamin B complex, vitamin C, 5 methyl folate, palmitoylethanolamide, bromelain, lycopene, sulforaphane, sodium alginate, magnesium, zinc, selenium, copper, and iron. In some embodiments individual ingredients may be substituted, dropped, and new ones added.

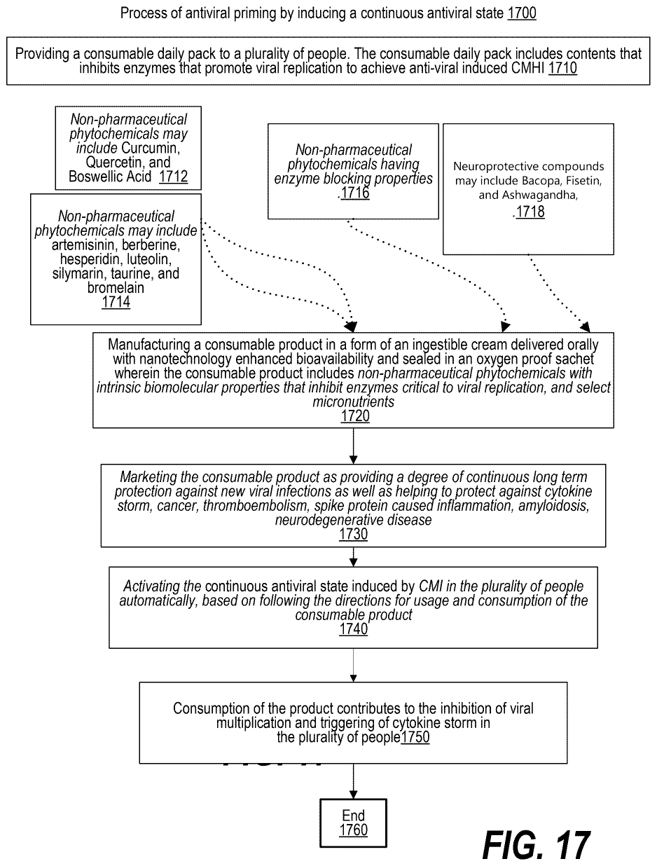

FIG. 17 depicts a process for antiviral priming by inducing a continuous antiviral state in a plurality of people 1700. At step 1710, the process provides a consumable daily pack to the plurality of people. The consumable daily pack includes contents that inhibit enzymes that promote viral replication to achieve antiviral priming induced CMHI.

At step 1720, a consumable product is manufactured in a form of an ingestible cream delivered orally with nanotechnology enhanced bioavailability and sealed in an oxygen proof sachet wherein the consumable product includes non-pharmaceutical phytochemicals with intrinsic biomolecular properties that inhibit enzymes critical to viral replication. The consumable product is designed for long term daily usage and includes neuroprotective compounds to help reduce or prevent cognitive impairment and neurodegenerative pathology. An objective for including contents in an embodiment for manufacturing the product for long term daily usage is to bundle in compounds that provide the most benefit with the least cost to be consumed by a plurality of people. The consumable product comprises non-pharmaceuticals and phytochemicals may include curcumin, quercetin, and boswellic acid 1712. The non-pharmaceuticals and phytochemicals have enzyme blocking properties 1714 with ingredients that may include artemisinin, berberine, hesperidin, luteolin, bacopa, fisetin, silymarin, taurine, and bromelain. The non-pharmaceuticals and phytochemicals may include properties selected from a group consisting of anti-cancer, neuroprotection, immunomodulatory, antioxidant, anti-prion, anti-amyloid, mitochondrial rehabilitation, and autophagy. The neuroprotective compounds 1718 may include Bacopa, Fisetin, Ashwagandha, Luteolin, Ginkgo biloba, Lutein, and Zeaxanthin. At step 1730, the consumable product is marketed as providing a degree of continuous long term protection against new viral infections as well as helping to protect against cytokine storm, cancer, thromboembolism, spike protein caused inflammation, amyloidosis, neurodegenerative disease. At step 1740, consumption of the product activates the continuous antiviral state induced by CMI in the plurality of people automatically, responsive to the plurality of people following the directions for usage and consumption of the consumable product. As a result of consumption of the product by the plurality of people, viral multiplication is inhibited and triggering of cytokine storm is reduced 1750. FIG. 17 processing thereafter ends at 1760.

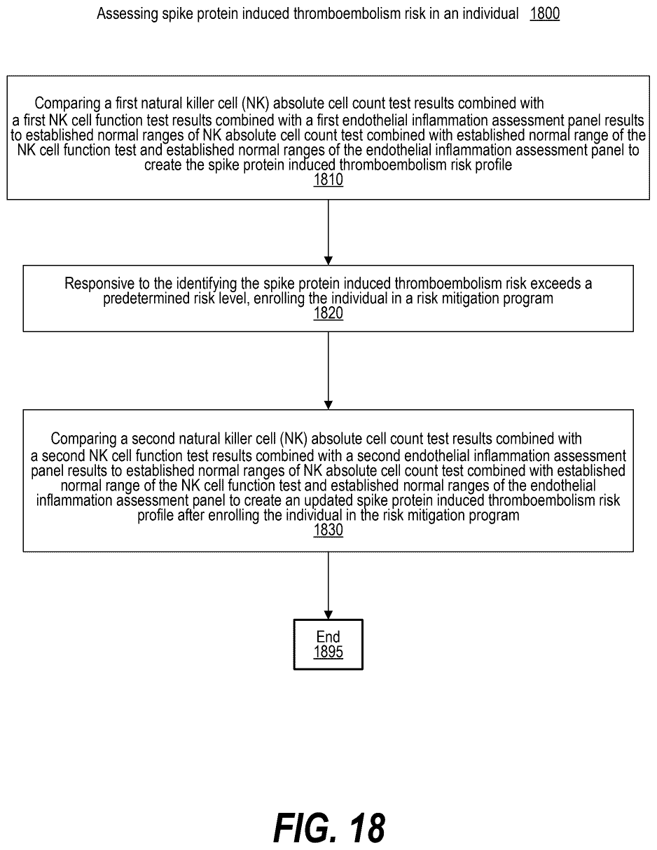

FIG. 18 shows the steps taken to assess spike protein induced thromboembolism risk in an individual 1800. At step 1810, the process compares a first natural killer cell (NK) absolute cell count test results combined with a first NK cell function test results combined with a first endothelial inflammation assessment panel results to established normal ranges of NK absolute cell count test combined with established normal range of the NK cell function test and established normal ranges of the endothelial inflammation assessment panel to create the spike protein induced thromboembolism risk profile. At step 1820, responsive to identifying the spike protein induced thromboembolism risk profile exceeds a predetermined risk level, enrolling the individual in a risk mitigation program. At step 1830, the process compares a second natural killer cell (NK) absolute cell count test results combined with a second NK cell function test results combined with a second endothelial inflammation assessment panel results to established normal ranges of NK absolute cell count test combined with established normal range of the NK cell function test and established normal ranges of the endothelial inflammation assessment panel to create an updated spike protein induced thromboembolism risk profile after enrolling the individual in the risk mitigation program. If the results of the test are in the established normal range, the risk mitigation program is considered successful for the treated individual. FIG. 18 processing thereafter ends at 1895.

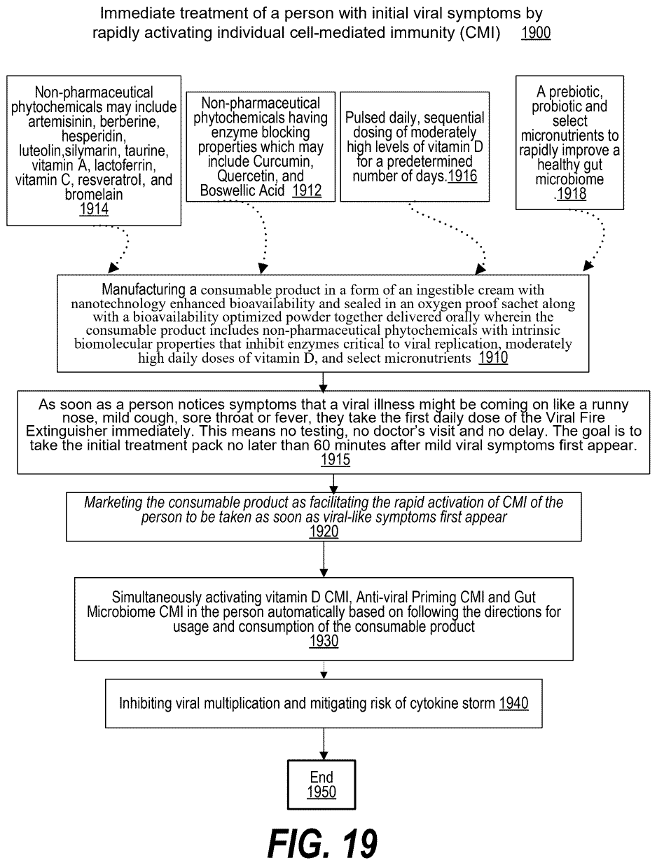

FIG. 19 shows the steps taken for immediate treatment of a person with initial viral symptoms by rapidly activating individual cell-mediated immunity (CMI) 1900. At step 1910, the process manufactures a consumable product in a form of an ingestible cream with nanotechnology enhanced bioavailability and sealed in an oxygen proof sachet along with a bioavailability optimized powder together delivered orally wherein the consumable product includes non-pharmaceutical phytochemicals with intrinsic biomolecular properties that inhibit enzymes critical to viral replication, moderately high daily doses of vitamin D, and select micronutrients. The non-pharmaceutical phytochemicals may include artemisinin, berberine, hesperidin, luteolin, silymarin, taurine, and bromelain 1914. The non-pharmaceutical phytochemicals may include curcumin, quercetin, and boswellic acid 1912. The non-pharmaceutical phytochemicals have enzyme blocking and immunomodulatory properties 1916 and may include pulsed daily, sequential dosing of moderately high levels of vitamin D for a predetermined number of days. The product may include a prebiotic, a probiotic and select micronutrients to rapidly improve a healthy gut microbiome 1918. At step 1920, the process markets the consumable product as facilitating the rapid activation of CMI of the person taking the consumable product as soon as viral infection symptoms occur. As soon as a person notices symptoms that a viral illness might be coming on like a runny nose, mild cough, sore throat or fever, they take the first daily dose of the Viral Fire Extinguisher immediately 1915. This means no testing, no doctor's visit and no delay. The goal is to take the initial treatment pack no later than 60 minutes after mild viral symptoms first appear. At step 1930, the process simultaneously activates vitamin D CMI, antiviral priming CMI and gut microbiome CMI in the person automatically based on following the directions for usage and consumption of the consumable product. As a result of activation of vitamin D CMI, antiviral priming CMI, and gut microbiome CMI, viral multiplication is inhibited and triggering of cytokine storm is reduced 1940. FIG. 19 processing thereafter ends at 1950.

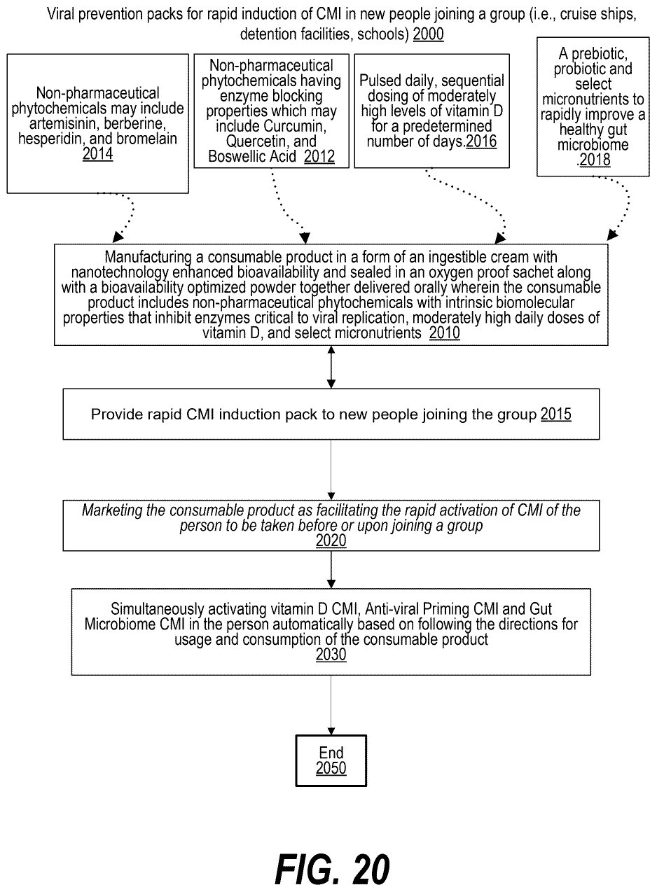

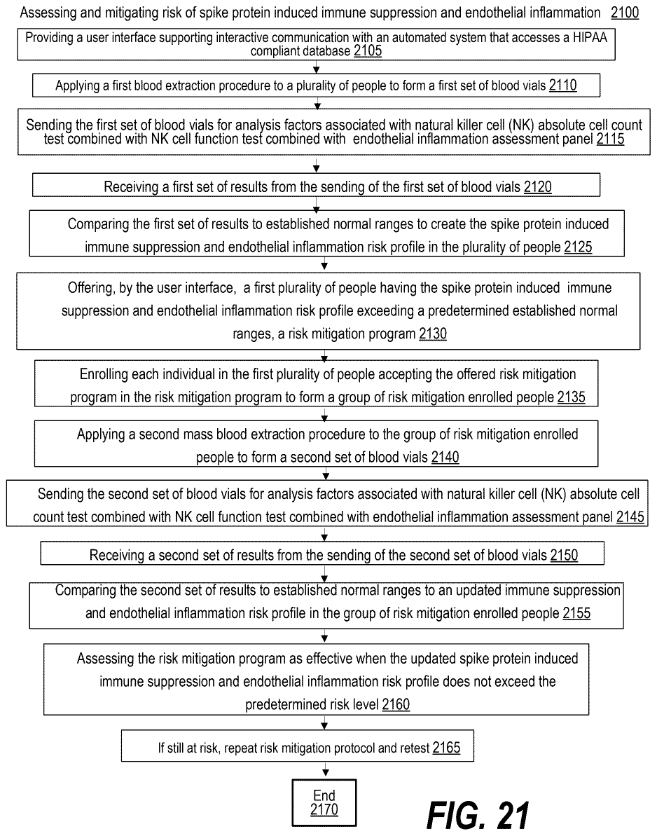

FIG. 20 shows the steps for using a viral prevention packs for rapid induction of CMI in new people joining a group (i.e., cruise ships, detention facilities, schools 2000. At step 2010, the process manufactures a consumable product in a form of an ingestible cream with nanotechnology enhanced bioavailability and sealed in an oxygen proof sachet along with a bioavailability optimized powder together delivered orally wherein the consumable product includes non-pharmaceutical phytochemicals with intrinsic biomolecular properties that inhibit enzymes critical to viral replication, moderately high daily doses of vitamin D, and select micronutrients. The consumable product is designed for short term daily usage intended to quickly activate the three elements of their personal CMI to contribute to CMHI for the health of the group. The prevention packs jumpstart the three forms of individual CMI that must be switched on before people enter a group indoors and which are key to CMHI. The product may include the non-pharmaceuticals and phytochemicals may include artemisinin, berberine, hesperidin, luteolin, silymarin, taurine, and bromelain 2014. The non-pharmaceuticals and phytochemicals may include curcumin, quercetin, and boswellic acid 2012. The non-pharmaceuticals and phytochemicals have enzyme blocking and immunomodulatory properties 2016 and may include pulsed daily, sequential dosing of moderately high levels of Vitamin D for a predetermined number of days. The product may include a prebiotic, a probiotic and select micronutrients to rapidly improve a healthy gut microbiome 2018. At step 2015, the process provides a rapid CMI induction pack to new people joining the group to achieve antiviral priming induced CMHI. At step 2020, the consumable product is marketed as facilitating the rapid activation of CMI to each person to be taken before or upon joining the group. At step 2030, the consumption of the product simultaneously activates vitamin D CMI, antiviral priming CMI and gut microbiome CMI in the person automatically based on following the directions for usage and consumption of the consumable product. FIG. 20 processing thereafter ends at 2050. FIG. 21 shows the steps taken by a process for assessing and mitigating risk of spike protein induced immune suppression and endothelial inflammation 2100. At step 2105, the process provides a user interface supporting interactive communication with an automated system that accesses a HIPAA compliant database. At step 2110, the process applies a first blood extraction procedure to a plurality of people to form a first set of blood vials. At step 2115, the process sends the first set of blood vials for analysis associated with natural killer cell (NK) absolute cell count test combined with NK cell function test combined with an endothelial inflammation assessment panel. At step 2120, the process receives a first set of results from the sending of the first set of blood vials. At step 2125, the process compares the first set of results to established normal ranges to create the spike protein induced immune suppression and endothelial inflammation risk profile in the plurality of people. The panels may include, for example, but not limited to Insulin Resistance Panel, Lipoprotein-associated phospholipase A2 Activity, Asymmetric dimethylarginine and Symmetric dimethylarginine, Myeloperoxidase, F2-Isoprostane/Creatinine Ratio, Oxidized low-density lipoprotein, Microalbumin, and Highly Sensitive C reactive protein.

At step 2130, the process offers, by the user interface, a first group of people having the spike protein induced immune suppression and endothelial inflammation risk profile exceeding predetermined established normal ranges, a risk mitigation program. At step 2135, the process enrolls each individual in the first group of people accepting the offered risk mitigation program in the risk mitigation program to form a group of risk mitigation enrolled people. At step 2140, the process applies a second mass blood extraction procedure to the group of risk mitigation enrolled people to form a second set of blood vials. At step 2145, the process sends the second set of blood vials for analysis associated with natural killer cell (NK) absolute cell count test combined with NK cell function test combined with an endothelial inflammation assessment panel. At step 2150, the process receives a second set of results from the sending of the second set of blood vials. At step 2155, the process compares the second set of results to established normal ranges to form an updated immune suppression and endothelial inflammation risk profile in the group of risk mitigation enrolled people. At step 2160, the process assesses the risk mitigation program as effective when the updated spike protein induced immune suppression and endothelial inflammation risk profile does not exceed the predetermined risk level. At step 2165, when the process assesses the risk mitigation program as not effective for an individual, that is, when the updated spike protein induced immune suppression and endothelial inflammation risk profile exceeds the predetermined risk level, the risk mitigation program may be adjusted or adapted for the person and after followed for some period of time retested. FIG. 21 processing thereafter ends at 2170.

FIG. 22 shows the steps taken by a company-specific viral safety certification program 2200. At step 2220, the process monitors, by an independent auditor, compliance of an implementation of four key interventions required to achieve CMHI by the company and wherein the four key interventions are enclosed space absolute humidity, serum concentration of vitamin D as measured by a blood test, healthful gut microbiome, and antiviral priming in individuals to determine a compliance assessment. Assessing, by the independent auditor, compliance levels associated with the four key interventions. Issuing the company-specific viral safety certification, by the independent auditor, based on the compliance assessment reaching a predetermined level of compliance. At predefined process 2230, the process performs the enclosed space absolute humidity routines (see FIG. 2-12 and corresponding text for processing details). At predefined process 2240, the process performs the serum concentration of vitamin D as measured by blood processes routines (see FIG. 13-14 and corresponding text for processing details). At predefined process 2250, the process performs the healthful gut microbiome processes routines (see FIG. 15-16 and corresponding text for processing details). At predefined process 2260, the process performs the antiviral priming processes routines (see FIG. 17-18 and corresponding text for processing details).

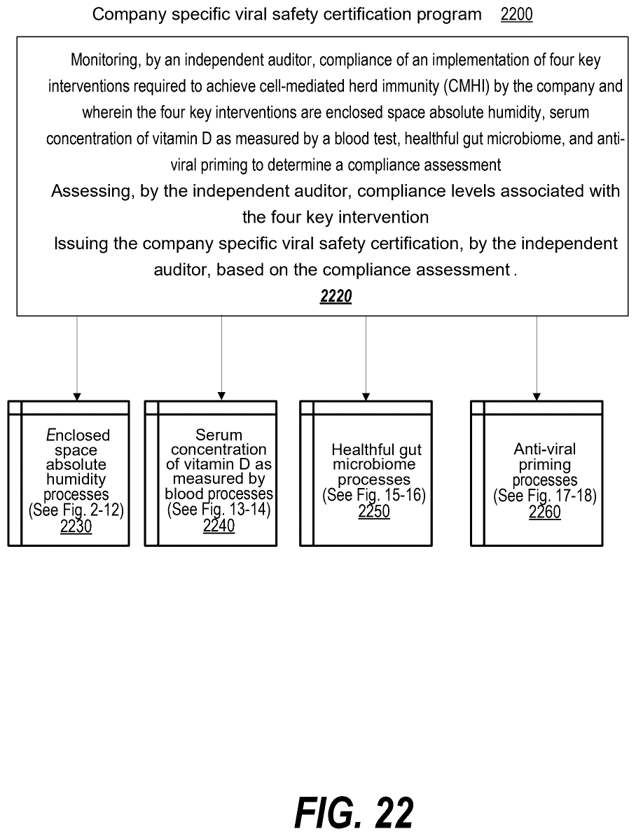

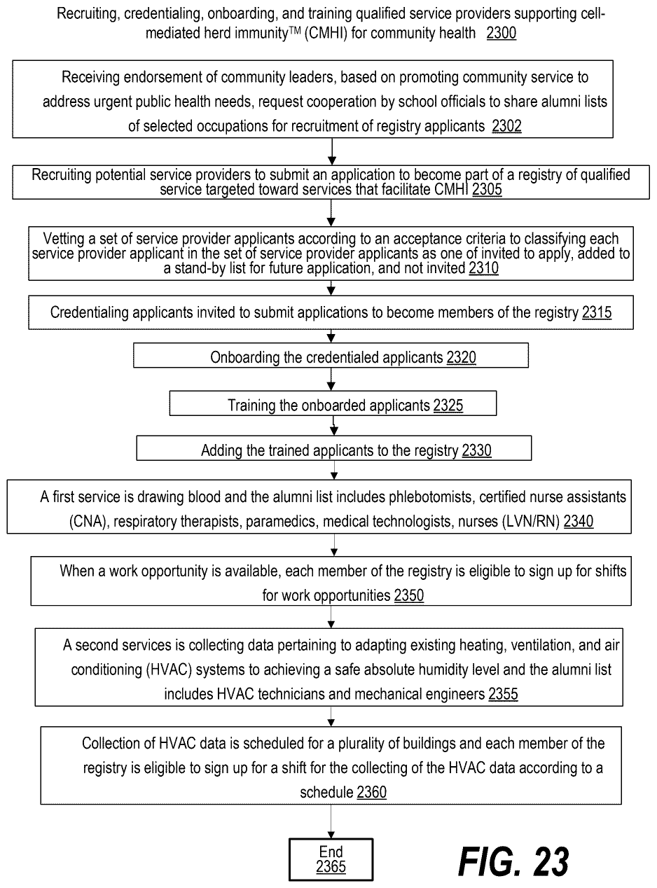

FIG. 23 shows the steps taken by a process that recruits, credentials, onboards, and trains qualified service providers supporting CMHI for community health 2300. At step 2302, the process receives endorsement of community leaders, based on promoting community service to address urgent public health needs, facilitating cooperation from college and technical school officials to share alumni lists of selected occupations for recruitment of registry applicants. At step 2305, the process recruits potential service providers to apply to become part of a registry of qualified service providers targeted toward services that facilitate CMHI. At step 2310, the process vets a set of service provider applicants according to an acceptance criteria to classifying each service provider applicant in the set of service provider applicants as one of invited to apply, added to a standby list for future application, and not invited. At step 2315, the process credentials applicants invited to submit applications to become members of the registry. At step 2320, the process onboards the credentialed applicants. At step 2325, the process trains the onboarded applicants. At step 2330, the process adds the trained applicants to the registry. A first service is drawing blood and the alumni list includes phlebotomists, certified nurse assistants (CNA), respiratory therapists, paramedics, medical technologists, nurses (LVN/RN) 2340. At step 2350, when a testing event occurs or a work opportunity is available, each member of the registry qualified to participate is eligible to sign-up for shifts for work opportunities. A second services is collecting data pertaining to adapting existing heating, ventilation, and air conditioning (HVAC) systems to achieving a safe absolute humidity level and the alumni list includes HVAC technicians and mechanical engineers 2355. At step 2360, collection of HVAC data is scheduled for a plurality of buildings and each member of the registry is eligible to sign-up for a shift for the collecting of the HVAC data according to a schedule. FIG. 23 processing thereafter ends at 2365.

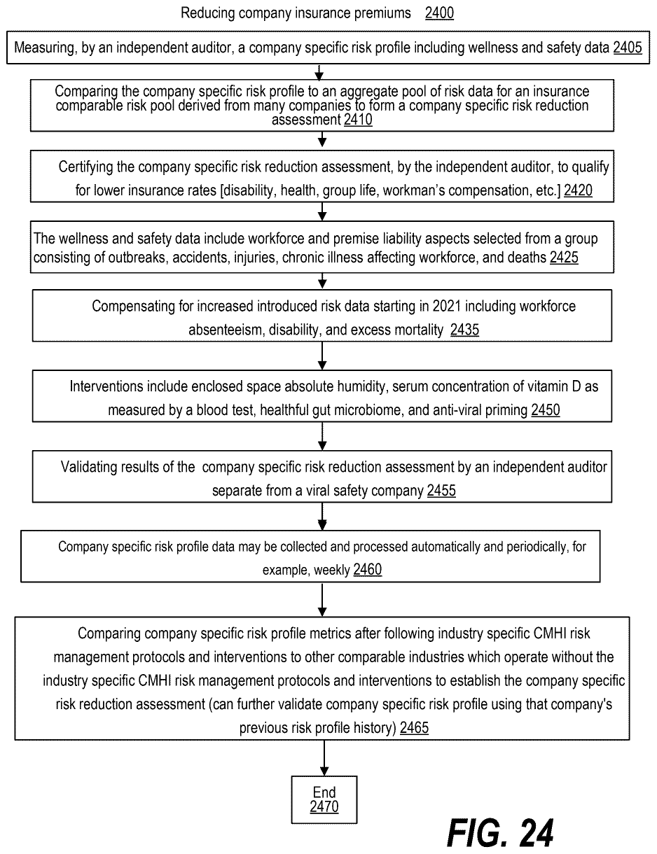

FIG. 24 shows the steps taken to reduce company insurance premiums 2400. At step 2405, an independent auditor establishes a company-specific risk profile including wellness and safety data. At step 2410, the process compares the company-specific risk profile to an aggregate pool of risk data derived from many companies to form a company-specific risk reduction assessment. At step 2420, the process certifies the company-specific risk reduction assessment, by the independent auditor, to qualify for lower insurance rates [disability, health, group life, worker's compensation, etc.]. At step 2425, the wellness and safety data include, but are not limited to, workforce and premise liability aspects selected from a group consisting of outbreaks, accidents, injuries, chronic illness affecting workforce [e.g., workforce productivity and absenteeism], and deaths. At step 2435, the process compensates for increased introduced risk data starting in 2021 including workforce absenteeism, disability, and excess mortality. At step 2440, the process improves the company-specific risk profile via industry specific CMHI risk management protocols and interventions. At step 2450, the process applies interventions which include enclosed space absolute humidity, serum concentration of vitamin D as measured by a blood test, healthful gut microbiome, and antiviral priming. At step 2455, the process validates results of the company-specific risk reduction assessment by an independent auditor separate from a viral safety company. At step 2460, company-specific risk profile data may be collected and processed automatically and periodically, for example, weekly. At step 2465, comparing company-specific risk profile metrics after following industry specific CMHI risk management protocols and interventions to other comparable industries which operate without the industry specific CMHI risk management protocols and interventions to establish the company-specific risk reduction assessment (can further validate company-specific risk profile using that company's previous risk profile history). FIG. 24 processing thereafter ends at 2470.

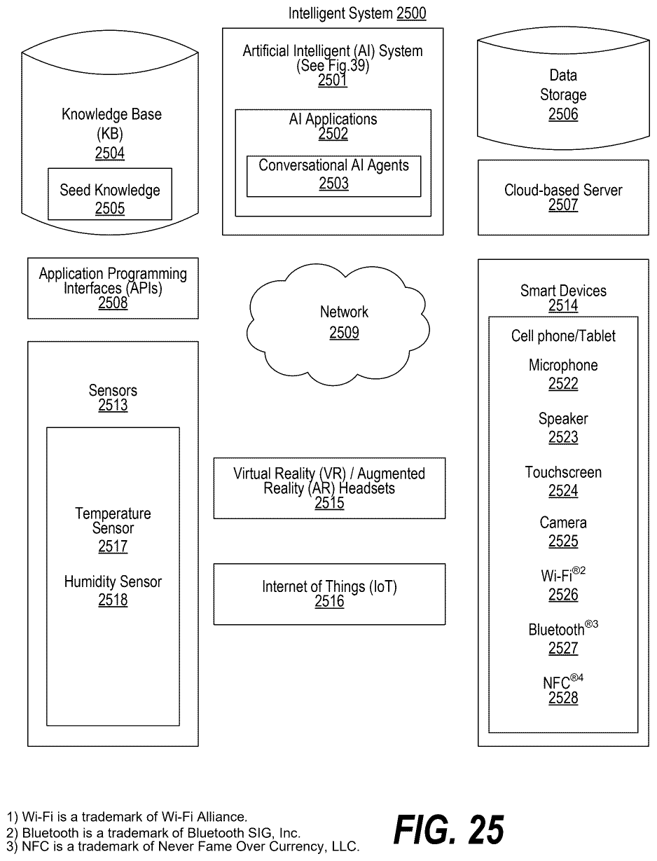

FIG. 25 depicts a high-level schematic representation of an intelligent system 2500 designed for facilitating contextually relevant conversational interactions.

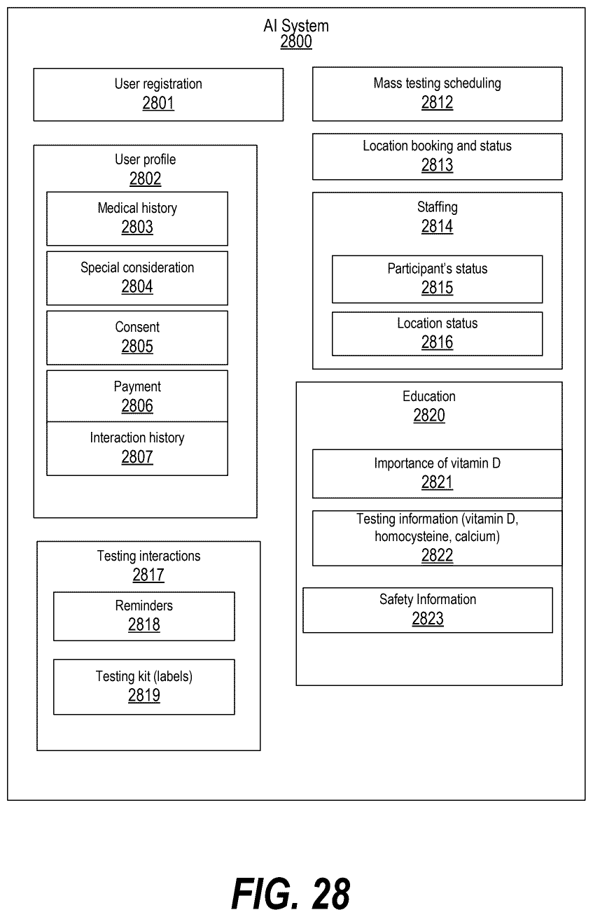

The intelligent system 2500 is a complex network of various technologies and components that work together to provide user-centered, contextually relevant, and personalized interactions. The intelligent system 2500 comprises an AI system 2501 (see FIG. 28 and corresponding text), a Knowledge Base (KB) 2504, and AI applications 2502, which can be conversational AI agents 2503.

The intelligent system 2500 also includes data storage 2506, cloud-based server 2507, application programming interfaces (APIs) 2508, and network 2509. The data storage 2506 stores and manages the vast amounts of data generated by the AI system 2501. The cloud-based server 2507 manages the processing and storage of data and provides computing power to enable the AI application to function. The network 2509 connects the various components and allows for communication between them.

The AI system 2501 can also integrate with various sensors 2513, smart devices 2514, virtual reality (VR)/augmented reality (AR) headsets 2515, and Internet of Things (IoT) 2516. To obtain contextual information about the environment, the system can use a range of sensors, including temperature sensor 2517, humidity sensor 2518. This information is then preprocessed and categorized before being displayed to users.

Moreover, smart devices 2514 come equipped with various features such as microphones 2522, speakers 2523, touchscreens 2524, cameras 2525, Wi-Fi®⁴ 2526, Bluetooth®⁵ 2527, and near field communications (NFC®⁶) 2528. These features enable the AI system to gather contextual information about the user's environment and interactions, allowing it to provide more personalized and relevant responses. For instance, the microphone can capture the user's voice input, while the camera can capture visual data such as facial expressions or object recognition. ⁴ Wi-Fi is a trademark of Wi-Fi Alliance.⁵ Bluetooth is a trademark of Bluetooth SIG, Inc.⁶ NFC is a trademark of Never Fame Over Currency, LLC.

The integration of various sensors 2513 and smart devices 2514 allows the AI system 2501 to gather and analyze data from multiple sources, thereby enabling it to detect contextual information about the environment. This, in turn, provides a more relevant and personalized user experience. The AI system 2501 can respond to user inputs and environmental changes through AI applications, creating a seamless and integrated user experience. By harnessing the power of virtual and augmented reality 2515, as well as the Internet of Things (IoT) 2516 devices, the AI system 2501 can further enhance its capability to detect and respond to user needs and preferences.

Referring to FIG. 25, the KB 2504 is a structured system, but not limited to a database, a set of databases, a repository, a set of repositories, and the like, which stores distinct types of data and diverse types of information that the AI system can access and use to provide contextually relevant and personalized responses.

The KB 2504 also manages information about the users. The information can be manually entered or automatically extracted from text, images, or other sources. The KB 2504 supports various applications such as object recognition, NLP, and decision making. The data and information stored in KB 2504 can be organized into categories, such as user properties, user attributes, user relations with the associated conversational AI agents 2503.

There are three main categories of data stored in the KB 2504. The first category is user attributes, which pertain to the physical characteristics demographic data that the AI system 2501 can utilize to interact with the users. The second category, environment data, pertains to the physical conditions in the environment, such as temperature, humidity, lighting, and other relevant factors, as well as the physical layout of the environment, including the location of doors, windows, and furniture. The third category is interaction data, which encompasses details about the user's preferences, previous interactions, and other relevant information.

User preferences may include information about their likes, dislikes, and other personal preferences that can be used to personalize the user's experience with the AI system. For example, if a user has expressed a preference for a particular type of music, the AI system can store this preference in the KB. The system can then use this information to recommend similar music or create a playlist based on the user's preferences. If the user previously interacted with the AI system and provided feedback on their experience, this information can also be stored in the KB. This information can be utilized to improve future interactions.

The AI system collects and stores data about the user's interactions with the system in the KB. By leveraging the data stored in the KB, the AI system can adapt to the user's preferences and behavior, creating a more seamless and intuitive conversational interaction. This personalized approach helps to build trust and engagement between the user and the AI system, leading to a positive user experience.

To add human-AI interaction data to the KB, the AI system initially processes the data to extract significant information such as user intents, behaviors, and preferences. This processing often involves using NLP techniques to parse the conversation and identify the essential elements. After the data has been processed, it is added to the KB in a structured format that is easily accessible and analyzed by the AI system. This format can include relevant contextual information such as the time and location of the interaction, the user's input, and the AI system's response.

Furthermore, the AI system may employ ML algorithms to analyze the interaction data and identify patterns and trends in the user's behavior and preferences. This analysis can then be used to refine the AI system's understanding of the user and improve its ability to produce personalized, contextually relevant responses. For instance, if the user frequently requests recommendations for local restaurants, the AI system can use this data to personalize future recommendations based on the user's past preferences and feedback. Using this approach, the AI system can provide contextually relevant and personalized recommendations, improving the overall user experience.

One important aspect of AI systems is their ability to integrate different data categories to gain a comprehensive understanding of the user's context and needs. This allows for user-centered, contextually relevant, and personalized conversational interaction. For example, the AI system can use the user's preferences and environment data to recommend activities or products suited to current conditions.

The KB can be updated manually or automatically depending on the specific implementation of the AI system. Manual updating of the KB typically involves human intervention, such as clients, a system administrator, or a data analyst adding or modifying information in the KB. This may be done through a user interface specifically designed for managing the KB, or through an API that allows direct programmatic access to the KB.

In the context of AI systems and software development, a client typically refers to an individual or organization that uses or purchases a product or service. The client may have specific requirements or expectations for the product or service, and they may provide feedback or input to the developers or providers to improve the product or service. In the case of an AI system, the client may be a business or individual who uses the system to perform tasks or gain insights. Typically, clients would not have direct access to update the KB themselves as it is a critical component of the AI system and requires specialized knowledge and expertise.

However, clients can indirectly update the KB by providing feedback and interacting with the AI system. For example, if a client's customized AI application is stored in the KB, the AI system can use the client's past interactions with the system to learn and update the KB accordingly. If the client provides feedback on their experience or preferences, this information can be used to adjust the AI system's understanding and improve its ability to provide relevant and personalized recommendations.

Additionally, in some cases, clients may have the ability to indirectly update the KB through a user interface or dashboard provided by the AI system. This interface may allow the client to adjust certain settings or preferences that can be stored in the KB and used to inform the AI system's responses and recommendations.

On the other hand, automatic updating of the KB occurs in real-time through the use of ML algorithms or other automated techniques. For example, an AI system may continuously monitor user interactions and automatically update the KB with added information learned from those interactions. The AI system may use natural language processing algorithms to extract relevant information from user inputs and use this information to update the KB in real-time.

One way that an AI system can identify added information from continuously monitoring user interactions is through ML algorithms. These algorithms can analyze substantial amounts of data from user interactions and identify patterns and trends that can be used to update the KB.

For example, the AI system is used to ensure all employees are participating in a company-specific CMHI risk management protocol. As users interact with the AI system and ask questions, the AI system can learn from these interactions and update its understanding of the types of questions being asked and the most appropriate responses to provide. The AI system can then use this updated information to improve future interactions and provide better support.

The AI system identifies relevant information through various techniques, such as natural language processing, data mining, and ML algorithms. Once the system has identified the relevant information, it processes the data to extract key insights and patterns that can be used to update the KB.

The process of updating the KB involves several steps. First, the AI system analyzes the new data to determine its relevance and significance to the existing knowledge base. The system then updates the KB with the added information, either by adding new entities or relationships or modifying existing ones.

To ensure the accuracy and reliability of the updated knowledge, the AI system may use techniques such as data validation and error correction. The system may also employ techniques such as differential privacy to protect sensitive information while still allowing meaningful insights to be drawn. Once the KB has been updated, the AI system can use the new knowledge to improve its performance in various tasks such as decision-making, natural language understanding, and predictive analytics. The updated KB allows the system to adapt to new situations and better understand user needs and preferences, leading to more accurate and personalized interactions.

To update the KB in real-time, the AI system can use automated processes that analyze and integrate added information as it is received. For instance, the AI system can be programmed to automatically update the KB when it detects new patterns or trends in user interactions.

Moreover, the AI system can utilize automated techniques to analyze data from diverse sources, such as social media, news feeds, and weather forecasts, to incorporate added information into the KB. For example, if the weather forecast predicts rain at a specific location, the AI system can access weather APIs to update the KB with this information. By integrating this updated data, the AI system can offer contextually relevant recommendations or actions to the user based on the current situation.

In one embodiment, a user interacts with a conversational AI agent through a shared ride driver's business card to request a ride from their current location to a testing destination. The conversational AI agent can use this interaction to update the KB, adjusting the user's preferences and attributes, such as their preferred car type, driver rating, and price range.

The iterative process can also help improve the accuracy of the relationship between the user and their past ride experiences, as well as the relationship between the user and other ride options in the area. For example, if the user has previously requested rides to a certain location during specific times of the day, the AI system can use this data to provide more accurate and relevant ride options in the future. The AI system can also analyze data from other sources, such as traffic patterns, weather conditions, and driver availability, to provide contextually relevant recommendations and adjust ride options accordingly.

In the described intelligent system 2500, the KB 2504 contains seed knowledge 2505 to establish and expand the KB. This initial set of information, data, or domain knowledge can include known facts, rules, relationships, and information about the environment that the AI system interacts with domain knowledge refers to the specialized understanding, insights, and expertise related to a specific area or field.

Additionally, domain knowledge can be contributed by a variety of sources, including subject matter experts, industry professionals, users, and other stakeholders. Users can also contribute domain knowledge through their interactions with the AI system. For example, a user could provide feedback on their preferences or experiences, which the AI system can then use to improve its responses and understand the user's needs.

An AI application can contribute domain knowledge to some extent, depending on the type and scope of the application. For example, an AI system that is designed to learn and improve over time, such as a ML system, can contribute to its own domain knowledge by analyzing data, detecting patterns and trends, and adjusting its responses accordingly.

Furthermore, an AI application that is designed to analyze and interpret substantial amounts of data, such as a predictive analytics system, can contribute domain knowledge by identifying correlations and making predictions based on its analysis.

In the development of the KB, domain knowledge serves as a solid foundation for the AI system to build upon. This foundation consists of relevant concepts, relationships, and rules specific to the domain. By incorporating domain knowledge as seed knowledge 2505, the AI system can start with a robust understanding of the subject matter, enabling it to generate accurate and contextually relevant responses. The AI system has mechanisms to gather, organize, and integrate this knowledge into the KB.

As the AI system interacts with users and acquires additional information, it can refine and expand its KB. During the training process, the seed knowledge is provided to a ML model, which enables it to learn and improve based on the initial data. By combining the seed knowledge 2505 with newly acquired data, the AI system becomes more proficient in its domain, improving its ability to provide meaningful and accurate responses, recommendations, or solutions.

Referring to FIG. 25, AI applications 2502 are specific implementations of the AI system designed to solve particular problems or perform specific tasks within a domain. These applications leverage the capabilities of the underlying AI system 2501 to provide tailored solutions for various industries and use cases. The relationship between an AI system and AI applications can be understood as a hierarchical structure where the AI system serves as the underlying foundation, and AI applications are built upon that foundation to provide specific functionalities and solutions.

In some embodiments, the AI application can be integrated with various smart devices and systems in a public service facility, such as a library or a government building. By accessing the data and functionality of other smart devices and systems, the AI application can provide an integrated user experience that is tailored to an individual's needs and preferences.

For instance, the AI application can integrate with sensors that detect the number of people present in the building and their locations, as well as with the building's HVAC system to regulate temperature, airflow, and absolute humidity. Using NLP and ML, the AI application can understand the preferences and behavior of visitors to the facility.

In some instances, AI applications manifest as conversational AI agents designed to interact with users through dynamic communication while simultaneously learning and adapting. By incorporating user feedback and interaction, these agents consistently improve their precision and effectiveness.

In an embodiment, an AI application 2502 running on a mobile device offers an intuitive interface that presents the gathered environment data in an accessible format for users. Environment data includes information about the environment surrounding the user, such as temperature, humidity, and lighting conditions, which can be used to provide more relevant and accurate responses. Besides showing the environment data, these devices also generate recommendations and suggestions derived from the collected information. For instance, if the AI application detects a low humidity in the user's surroundings and identifies that user is indoor, the AI application might advise the user to leave the premises by sending a push notification through the mobile device or in some environment the AI application may be able to turn on the humidify that is located near the user.

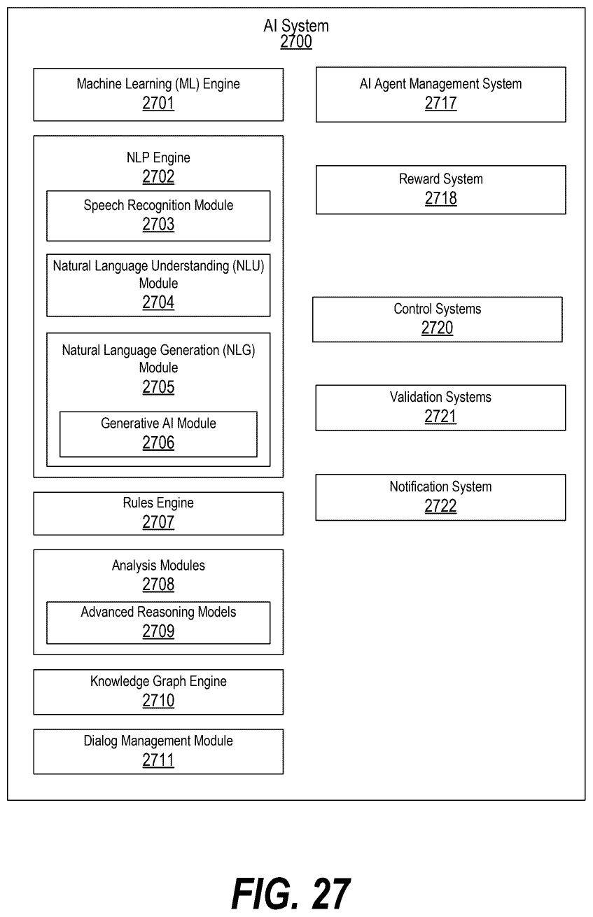

Referring to FIG. 25, the AI system 2501 (See FIG. 27) serves as the principal component of the intelligent system, employing a range of techniques like NLP, ML, and advanced reasoning to predict user intents and objectives.

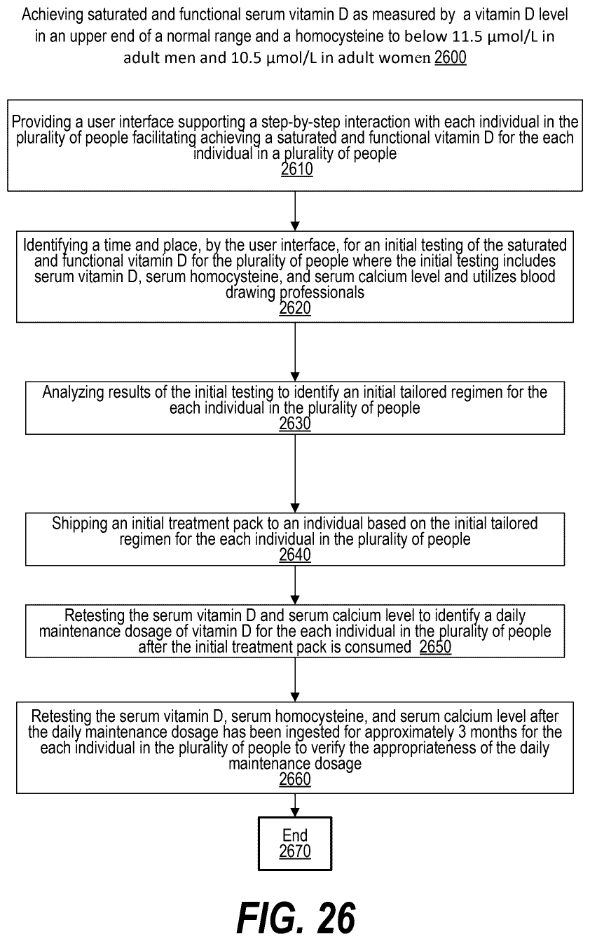

FIG. 26 depicts a process for achieving saturated and functional serum vitamin D as measured by a vitamin D level in an upper end of a normal range and a homocysteine to below 11.5 μmol/L in adult men and 10.5 μmol/L in adult women 2600. At step 2610, the process provides a user interface supporting a step-by-step interaction with each individual in a plurality of people facilitating achieving a saturated and functional vitamin D for the each individual in a plurality of people. At step 2620, the process identifies a time and place, by the user interface, for an initial testing of the saturated and functional vitamin D for the plurality of people where the initial testing includes serum vitamin D, serum homocysteine, and serum calcium level and utilizes blood drawing professionals. At step 2630, the process analyzes results of the initial testing to identify an initial tailored regimen for the each individual in the plurality of people

At step 2640, the process ships an initial treatment pack to each individual based on the initial tailored regimen for each individual in the plurality of people. At step 2650, the process re-tests the serum vitamin D and serum calcium level to identify a daily maintenance dosage of vitamin D for each individual in the plurality of people after the initial treatment pack is consumed. At step 2660, the process retests the serum vitamin D, serum homocysteine, and serum calcium level after the daily maintenance dosage has been ingested for approximately 3 months for each individual in the plurality of people to verify the appropriateness of the daily maintenance dosage. FIG. 26 processing thereafter ends at 2670.

FIG. 27 depicts a high-level block diagram illustrating components of an AI system 2700 for contextually relevant conversational interaction in the environment. The AI system includes a ML engine 2701, an NLP engine 2702, a speech recognition module 2703, a natural language understanding (NLU) module 2704, a natural language generation (NLG) module 2705, a generative AI module 2706, a rules engine 2707, analysis modules 2708, advanced reasoning models 2709, a knowledge graph engine 2710, a dialogue management module 2711, an AI agent management system 2717, a reward system 2718, control systems 2720, validation systems 2721, and a notification system 2722, to provide user-centered, contextually relevant, and personalized interaction in the environment.

The ML engine 2701, NLP engine 2702, and analysis modules 2708 are important components that enable and improve the AI system's abilities of “thinking and acting” logically to achieve the best outcome. These components collaborate to predict user intents, behaviors, and conversation topics effectively.

The ML engine 2701 included in the AI system is a core component responsible for developing, training, and deploying ML models to solve specific problems or perform tasks, such as classification, regression, or clustering. The ML engine 2701 plays a key role in the AI system by enabling it to learn from data, adapt to new inputs, and make data-driven decisions or predictions.

The ML engine 2701 typically comprises of the following key elements: (1) Algorithms: The ML engine uses a variety of ML algorithms, such as decision trees, support vector machines, neural networks, or clustering algorithms, to build models based on the data provided. The choice of algorithm depends on the problem being addressed, the nature of the data, and the desired level of accuracy and interpretability. (2) Data Preprocessing: The ML engine includes preprocessing techniques to clean, transform, and preprocess raw data, making it suitable for use with ML algorithms. This step may involve data cleaning, normalization, feature engineering, and feature selection. (3) Model Training: The ML engine uses a training dataset to train the selected algorithm, adjusting its parameters or weights to minimize the error between the model's predictions and the actual output data. The training process can involve techniques like gradient descent, backpropagation, or other optimization methods. (4) Model Validation and Evaluation: The ML engine assesses the performance of the trained model using a validation dataset, allowing model tuning and preventing overfitting. Evaluation metrics, such as accuracy, precision, recall, or F1 score, help quantify the model's performance. (5) Model Deployment: Once the model has been trained and validated, the ML engine deploys the model within the AI system, enabling it to make predictions or perform tasks based on new, unseen data. (6) Model Updating: The ML engine continually monitors the model's performance, updating it as needed to account for changes in the data or problem domain. This process can involve retraining the model with new data, adjusting its parameters, or replacing it with a new model if required.

Referring to FIG. 27, the NLP engine 2702 is designed to receive and process user input in natural language by performing various NLP tasks, which may include parsing, part-of-speech tagging, sentence breaking, stemming, word segmentation, terminology extraction, grammar induction, lexical semantics, machine translation, named entity recognition (NER), NLG, NLU, and relationship extraction, among others.

By employing techniques such as language modeling and text classification, the NLP engine generates contextually relevant responses based on user objectives, context, and current state. The NLP engine 2702 analyzes user input using NLP algorithms to understand the meaning and intent behind the user's message. The NLP engine may also apply advanced topic mining and modeling techniques to enhance the accuracy of NLU.

NLU is a subfield of NLP that focuses on enabling computers to comprehend and interpret human language as it is spoken or written. NLU goes beyond simply recognizing the words or phrases used in a text or speech; it seeks to understand the underlying meaning, context, and intent of the language, just as a human would.

NLU typically involves several tasks and processes, such as: (1) Tokenization: Breaking down text or speech into individual words, phrases, or other meaningful units called tokens. This step enables the AI system to analyze the language at a more granular level. (2) Part-of-speech tagging: Assigning grammatical categories to each token, such as nouns, verbs, adjectives, and so on. This helps the AI system understand the role and function of each word in a sentence. (3) Syntax analysis: Analyzing the grammatical structure of a sentence to determine the relationships between words and phrases. This helps the AI system understand how the various parts of the sentence fit together to convey meaning. (4) Semantic analysis: Identifying the meaning of individual words and phrases, as well as the overall meaning of the sentence, considering factors such as word sense disambiguation, idiomatic expressions, and context. (5) Discourse analysis: Understanding the relationships between sentences and the broader context of the text or conversation, such as determining the references to pronouns or recognizing the purpose of a discourse. (6) Sentiment analysis: Identifying the emotions, opinions, or attitudes expressed in the language, which can be useful for applications such as social media monitoring, customer feedback analysis, and market research. (7) Intent recognition: Determining the user's goal or intention in a given conversation or interaction, which is particularly important for chatbots and virtual assistants.

The NLU module 2704 in the AI system 2700 is designed to process and interpret human language, especially in the context of conversational agents, chatbots, and other natural language processing applications. The NLU module allows AI systems to comprehend the meaning and intent behind textual input, enabling effective communication between the system and the user.

In the AI system 2700, the NLU module 2704 starts by preprocessing the raw textual input. This process may involve tokenization, which breaks the text into words or tokens, lowercasing, removing special characters, and stemming or lemmatization, which reduces words to their root form. Following preprocessing, the module extracts various features from the text, such as word frequency, word embeddings, or other language-specific characteristics. These features help represent the input text in a structured format that can be understood by the AI system.

To identify the intent or purpose behind the user's input, the NLU module analyzes the extracted features. Intent recognition may employ ML techniques like classification algorithms or rule-based methods that map specific patterns in the input text to predefined intents. Along with recognizing intent, the NLU module extracts relevant entities or information from the input text. Entities can consist of dates, times, locations, names, or any other information pertinent to the interaction context. Entity extraction techniques can include named entity recognition, regular expressions, or custom algorithms tailored to the AI system's specific domain.

After identifying intents and extracting entities, the NLU module integrates this information into the context of the ongoing conversation or interaction. The context helps the AI system better understand the user's needs, preferences, or goals, allowing it to generate appropriate responses or actions. Lastly, the NLU module outputs the interpreted information, including the recognized intent, extracted entities, and context, to other components of the AI system. This information is utilized by modules like the Natural Language Generation module, context-aware modules, or decision-making components to generate contextually relevant responses or actions.

The Natural Language Generation (NLG) module 2705 is also a key component of the AI system 2700 designed for conversational interactions. The NLG module is responsible for creating coherent, human-like text responses based on the input and context provided by other components of the AI system, such as NLU and context-aware modules. The NLG module enables AI systems to generate responses that are easily understood by users, facilitating effective communication and improving the overall user experience.

Generative AI is a subfield of artificial intelligence that focuses on the creation of added content or data, such as text, images, or audio, based on input data and context. This is achieved through advanced ML techniques, such as deep learning and neural networks. Generative AI models, such as Generative Adversarial Networks (GANs) or Variational Autoencoders (VAEs), can generate realistic and high-quality outputs by learning complex patterns and structures from large datasets during the training process.

Integrating the Generative AI module 2706 within the NLG module can significantly enhance the capabilities of the AI system in generating contextually relevant, natural-sounding text responses during conversational interactions. The Generative AI can leverage its ability to learn complex patterns and structures from large language datasets to produce human-like responses that are not only coherent but also tailored to the specific context of the interaction.

By incorporating the Generative AI module 2706 into the NLG module 2705, the AI system can streamline the process of generating human-like responses. This is achieved by utilizing the Generative AI's capabilities to analyze and generate text based on the input and context provided by other system components, such as NLU and context-aware modules. The Generative AI can then produce contextually appropriate responses that align with the user's intent and the ongoing conversation, resulting in more effective communication.

Furthermore, the integration of Generative AI within the NLG module allows the AI system to leverage the Generative AI's advanced learning capabilities to continuously improve its performance over time. As the Generative AI is exposed to more data and diverse conversational contexts, it can refine its understanding of language patterns, enabling the generation of increasingly accurate and contextually relevant responses.

The incorporation of Generative AI within the NLG module of an AI system can significantly enhance the system's ability to generate contextually relevant, natural-sounding text responses during conversational interactions. This integration streamlines the response generation process, leverages the Generative AI's capabilities to improve the overall effectiveness of the NLG module. This results in a more engaging and satisfying user experience.

Referring to FIG. 27, the NLG module 2705 is responsible for creating natural and fluent text or speech from structured information or data. The NLG plays a key role in the field of NLP and AI applications, as it allows machines to produce output that is not only understandable by humans, but also contextually appropriate, grammatically accurate, and logically organized.

The NLG module 2705 typically involves several stages to generate text or speech output. The first stage is content determination, where the AI system identifies important information or data points based on the context and purpose of the generated text. The second stage is discourse planning, where the selected information is organized into a logical structure to create a coherent narrative. The third stage is sentence planning, where appropriate sentences are generated to convey the selected information effectively and naturally. The final stage is realization, where the planned sentences are converted into final text or speech output, adhering to the rules and conventions of the target language, and including appropriate formatting elements and intonation for speech output.

The stages described for the NLG module are typically performed in the order presented: content determination, discourse planning, sentence planning, and realization. However, the specific implementation of NLG can vary depending on the system and task at hand, and some steps may be combined or performed in a different order.

For example, some NLG systems may use a data-to-text approach, where the content determination and sentence planning stages are combined into a single step that involves mapping input data to natural language sentences. In other cases, the discourse planning stage may be more complex, involving the generation of multiple paragraphs or sections with different structures or styles.

In some embodiments, the AI system may use NLP tasks and methods to generate dynamic responses to user questions that do not have predefined answers in KB. The NLG module can be used to generate text or speech output based on the selected information and the context of the user's query. The use of NLP techniques allows the AI system to understand the user's intent and extract relevant information from their query, enabling the generation of accurate and contextually relevant responses.

For example, the AI system may employ an NLP technique called named entity recognition (NER) to identify key entities in the user's query, such as the names of people or places. The system may then use this information to generate a response that is personalized and contextually relevant to the user's query. Alternatively, the AI system may use an ML algorithm to generate responses based on patterns in the user's queries and past interactions with the system. The system may learn from the user's previous queries and responses to improve the accuracy and relevance of its responses over time.

In certain embodiments, the AI system may encounter user inquiries that have no predefined responses in the KB. For example, a user could pose a question to an AI-powered virtual assistant, such as “What is the optimal time to have my next test in Hawaii?” Although the AI system may have some general knowledge about Hawaii, it may lack a specific response for the user's query.

To address this, the AI system may utilize NLP techniques to decipher the user's intent and extract pertinent information from the query. The system could recognize that the user has a yearly vitamin D test that is due and evaluate terms such as “optimal time” and “Hawaii” and deduce that the user is seeking information about mass testing scheduled in Hawaii. The NLG module may then generate a tailored response based on this interpreted intent, such as “According to our data, the next mass scheduled test is on the <specific date>currently scheduled.” The NLG module can generate a dynamic response based on the analysis and recommendations from other components like the rules engine, knowledge graph, and analysis modules, in addition to NLP results.

Referring to FIG. 27, the speech recognition module 2703 is a key component of the NLP engine, as it allows the AI system to process spoken language input from users. The speech recognition module 2703 is responsible for converting spoken language into written text or interpreting specific voice commands. The speech recognition module 2703 is responsible for managing the entire speech recognition process, from input and preprocessing to output generation.

Speech recognition algorithms are responsible for understanding the acoustic features and linguistic patterns in the audio input to generate the desired output, such as text or commands, wherein speech recognition algorithms can be applied to convert the audio input into text format, which can then be processed by the NLP engine using various techniques such as sentiment analysis, entity recognition, and text classification. The output from the NLP engine can then be used to generate spoken language output using text-to-speech technology. So, speech recognition algorithms and NLP techniques are often used in combination to enable natural language interaction between humans and machines.

Additionally, the speech recognition algorithms typically involve ML techniques, statistical models, and other advanced processing methods that help the AI system accurately identify and transcribe spoken language.

In certain embodiments, when a user provides a voice command as input, the conversational AI agents utilize the speech recognition module and one or more speech recognition algorithms to convert the user's voice input into plain text. This text can then be parsed and processed by the NLP engine to generate structured data for analysis. The speech recognition algorithms play a key role in the speech recognition module by analyzing and recognizing human speech.

The rules engine 2707 in the AI system is a key component responsible for managing, processing, and applying a predefined set of rules or logic to the AI system. The rules engine 2707 is designed to evaluate complex conditions, make decisions, and execute actions based on these rules. The rules engine helps automate decision-making processes, ensuring consistent and accurate outcomes while reducing the need for manual intervention.

In the context of the AI system, the rules engine can work alongside ML and NLP components to enhance the AI system's overall intelligence and adaptability. The rules engine can be used to: (1) Define and enforce domain-specific constraints: By incorporating expert knowledge or industry-specific guidelines into the rules engine, the AI system can adhere to specific requirements or standards, thus ensuring the AI system's output is compliant and relevant. (2) Implement business logic: The rules engine can be used to apply business rules or policies consistently across the AI system's various tasks and processes, ensuring that the AI system's actions align with the organization's objectives and priorities. (3) Control the AI system's behavior: By setting and adjusting rules in the rules engine, developers or administrators can easily configure the AI system's behavior, tailoring it to the specific needs of the users or the application. (4) Improve interpretability and transparency: Rules-based systems can offer a higher degree of explain-ability compared to some black-box ML models, as the decision-making process is based on explicit rules that can be understood and audited. (5) Complement ML models: In some cases, combining rules-based logic with ML models can lead to a more robust and accurate AI system. The rules engine can handle scenarios where ML models may struggle, such as situations with limited training data or those requiring strict adherence to specific regulations.

FIG. 28 depicts an embodiment of an Artificial Intelligence (AI) system trained to support mass testing. The AI system 2800 supports user registration 2801 where individuals sign-up and responsive to signing up, the AI system 2800 creates a user profile 2802. The AI system 2800 interacts with the user to collect information related to the user. In an embodiment, the user may fill out a health questionnaire with information to be supplied by the user. The requested information would include demography, age, height, weight, sex so body mass index (BMI) could be calculated. Also, information such as any previous relevant illness such as sarcoidosis or parathyroid disease, chronic kidney disease, and the like. In some embodiments, information may be retrieved from other electronic data records, such as, patient portals, which could include primary care physicians, specialists, lab testing results, and the like. The information may be classified as medical history 2803 and could include various categories, such as, special considerations 2804. Information may include whether the user is a “hard stick,” i.e., difficult to draw blood, etc. The AI system requests the user's consent 2805 to the testing and provides information, such as, privacy information, what information is shared and at what granularity. The AI system requests payment from the user and records the payment 2806 when received. Besides testing for vitamin D, the AI system may provide options for the user have additional testing, for example, but not limited to, Virus Acquired Immune Deficiency (VADS). The AI system keeps track of communications to and from the patient as an interaction history 2807.

The AI system prompts the user who may be identified as a participant or a patient to select a date for testing. Proposed dates may be processed by a mass testing scheduling 2812 component of the AI system. Many factors could influence the choice for location booking 2813. Getting workers, that is, staffing 2814 who could draw blood to participate is also a part of the mass testing scheduling 2812 infrastructure. The AI system provides testing interactions 2817 with each individual participant and keeps track of each participant's status 2815 as well as keeping track of the location status 2816. The testing interactions 2817 includes reminders 2818 which include such information as to whether the user should fast, what materials need to be brought, when to arrive, etc. In an embodiment, the user is mailed labels to be supplied to the person drawing the blood. The AI system would interact with the user with reminders 2818 of what needs to be brought, the place to go for testing, and any other information helpful for facilitating an expedited processing of the testing. For example, the user may be reminded to bring the envelope with the quick response (QR) codes to the scheduled mass testing 2812. The envelope may include testing kit sticky labels 2819 to be given to a blood drawing person. Included in the education 2820 provided by the AI system 2800 is information about the importance of vitamin D 2821, testing information identifying that vitamin D, homocysteine and calcium 2822 are part of the initial testing and safety information 2823 regarding the level of vitamin D as well as potential problems related to out of normal range values for magnesium or calcium. The user may be asked to decide on any other testing option, such as, for example, but not limited to epithelial disease, vaccine induced immune deficiency, and the like. If the user requests addition testing, then the user is instructed to include the desired sticky labels in the envelop for the testing. When the user arrives at the testing site, the user's envelop with the QR codes are scanned in and, if the user arrived in the allocated time slot, directed to a table to get blood drawn. Periodically, vials of blood may be carried to a fulfillment center for partial processing, for example, centrifuged, and express mailed for lab testing. Processing of each vial may be automatically tracked based on a stock keeping unit (SKU) on each label with communication with the user. If the results of the lab analysis for initial vitamin D is normal, the system identifies the ramp up dosage and a maintenance dosage, prepares the ramp up dosage, and mails the ramp up dosage to be taken over a first period of time. In one embodiment that first period of time is 20 days where the user is instructed to purchase and consume a maintenance dosage. In some embodiments, a maintenance dosage is also supplied for a second period of time. In an embodiment ramp up is 10 days followed by 10 days maintenance or plateau. Typically, a second blood draw is taken after about 4 weeks to verify blood serum levels are in an upper range. In an embodiment, after taking the maintenance dosage of vitamin D for 3-4 months, a second blood draw is taken to verify the maintenance dosages keeps the patent in the upper range for vitamin D. In an embodiment, maintenance testing is performed yearly and is scalable to large populations. Dosages are based on BMI and communication via AI application. Epigenetic factors affecting binding of proteins are tracked using a quantitative model that checks before and after consumption of the supplied packets. Patient information and results are sent to the AI application to facilitate increased accuracy over time. Expect under 1% of those undergoing vitamin D treatment would need a second treatment pack. If the user arrives after the allocated time slot, the user may be directed to a waiting line.

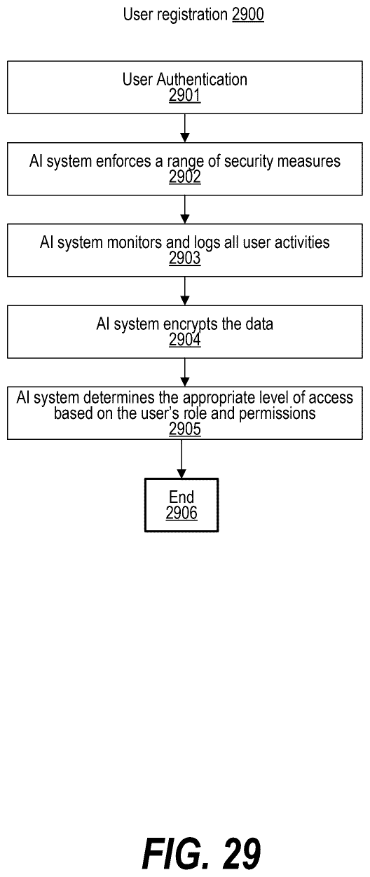

FIG. 29 shows taken by a user registration process 2900. At step 2901, the user is authenticated. This may involve supplying identification, submitting personal information, and even video authentication. At step 2902, the AI's system enforces a range of security measures which may include, for example two factor authentication. At step 2903, the AI system may monitor and log all user activities. The activity may be associated with a user profile created when the user successfully registers and included in a knowledge base (KB). At step 2904, the AI system encrypts any sensitive information or data pertaining to the user. At step 2905, the process AIs system determines the appropriate level of access based on the user's role and permissions. In cases where a user is a participant of mass testing, the user is allowed to communicate with the AI system, but not allowed to directly administer any changes other than via communication with the AI system. FIG. 29 processing thereafter ends at 2906.

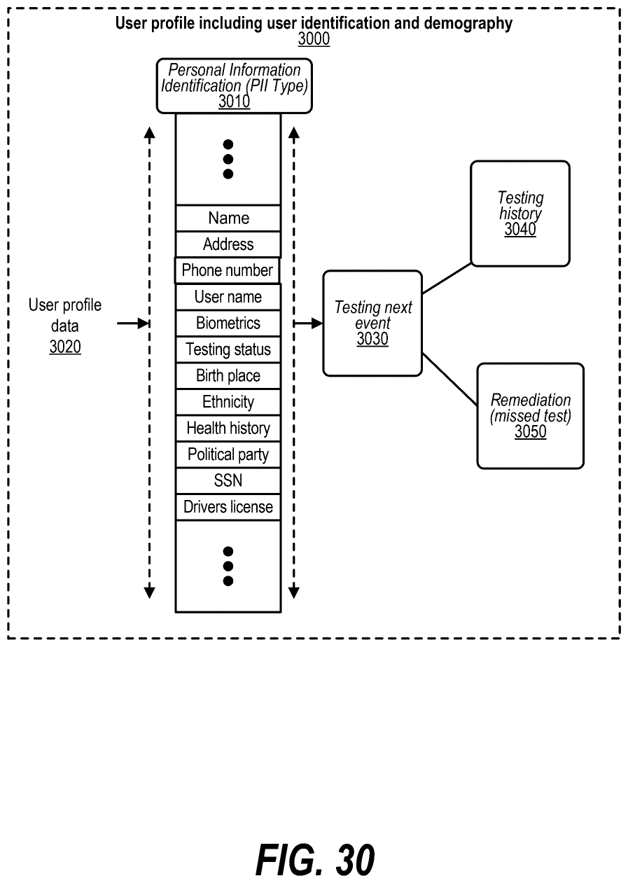

FIG. 30 depicts a representation of a user profile data which includes user identification and demography 3000. In an example embodiment, a user profile data infrastructure pointer 3020 points to a structure that identifies a Personal Information Identification Type (PII) 3010. The structure includes next event testing 3030, testing history 3040, and remediation (missed test) history 3050. In many cases, the information and sensitivity of fields in the user profile data are known, such as, when the user fills out a form. In some embodiments, the data may be included via other approaches in which case support may be provided utilizing a field recognition. Many distinct types of categories may be supported, such as, but not limited to, not sensitive, sensitive personal based on discovery, mild sensitive personal, medium sensitive personal, extremely sensitive personal, business sensitive, business confidential, and the like. There are many ways that the information may be classified and/or identified. In some embodiments the fields may be known based on a template in a form, a user classification, a scan using regular expression, etc. Having personal information available in one or more files related to a single person may change or affect the sensitivity of the information in the files. For example, being able to identify the specific person for which the information refers may be considered highly sensitive depending on how the data is used. Information in the metadata identifies how user data may be used and tracks copies of user data.

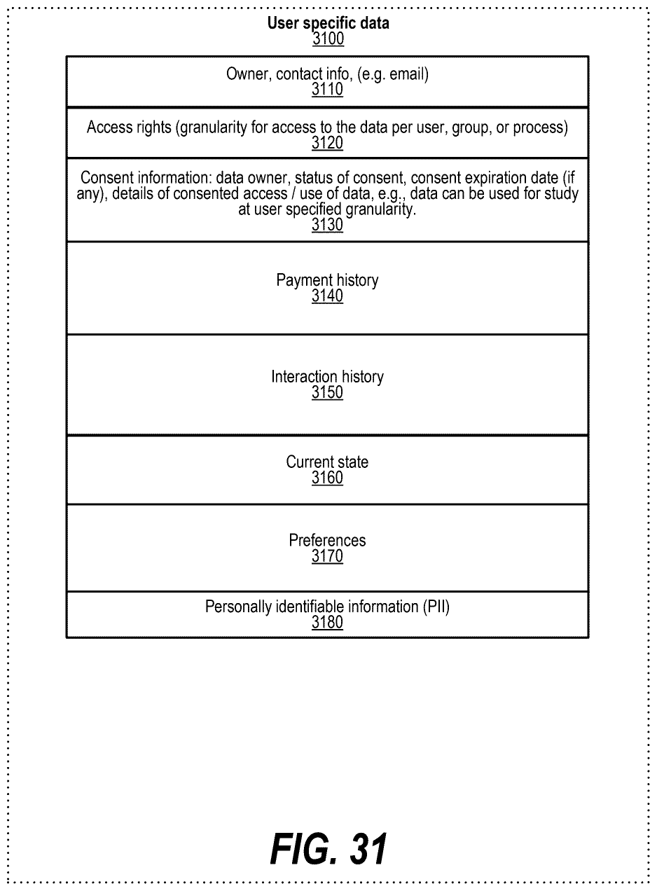

FIG. 31 depicts a high-level representation user specific data 3100. The data includes: User's contact information, e.g. email 3110. Access rights (granularity for access to the data per user, group, or process) 3120. Consent information: data owner, status of consent, consent expiration date (if any), details of consented access/use of data, e.g., data can be used for study at user specified granularity 3130. Payment history 3140. Interaction history 3150. Current state 3160, for example, awaiting test results. Preferences 3170, for example, prefers instant messages. Personal identifiable information (PII) 3180 which includes discovery and mapping details. In an embodiment, all access to file data is audited, such that, when a selected file is accessed, the auditing information records information related to accessing the selected file, who accessed the selected file, when the selected file is accessed, and any actions performed on the selected file. The consent information includes a purpose for which the personal data can be used, a date that an authorization was given by the data owner, and a date of the expiration. The selected file may be deleted automatically after the date of the expiration. The system may automatically adjust the user specific data based on current contents in the file and a current consent information of the data owner/user.

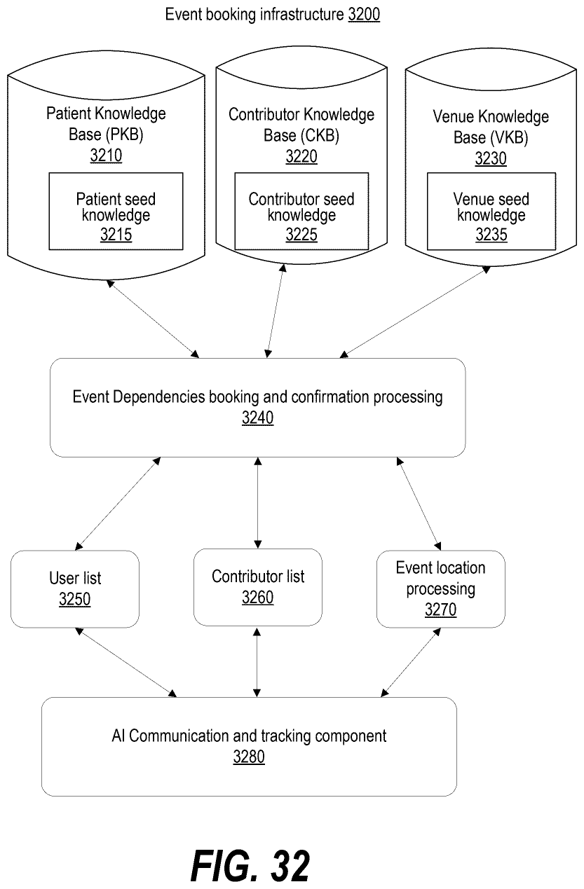

FIG. 32 depicts a schematic view of an event booking infrastructure 3200. In an embodiment an Artificial Intelligence (AI) event scheduling prediction model is used. The AI knowledge base may be represented by a patient knowledge base (PKB) 3210, a contributor knowledge base (CKB) 3220, or a venue knowledge base (VKB) 3230. Event dependencies booking and confirmation processing 3240 takes as input a list of users expected to participate in a testing event 3250, a list of service providers eligible to administer the tests 3260, and event location processing or booking status 3270 updated real-time by the AI communication and tracking component 3280. The AI event scheduling prediction model determines a number of predicted participants, a number of predicted test administers, and an availability of the event location to assess a probability of holding a successful testing event on a specific day. If the probability exceeds a predetermined success threshold, the event is tentatively scheduled. The probability of the number of predicted participants is calculated from the patient knowledge base (PKB) 3210 which was initialized with patent seed knowledge 3215. The probability of the number of predicted test administrators or contributor is calculated from the contributor knowledge base (CKB) 3220 which was initialized with contributor seed knowledge 3225. The probability of the event location booking, or venue is calculated from the venue knowledge base (VKB) 3230 which was initialized with venue seed knowledge 3235.

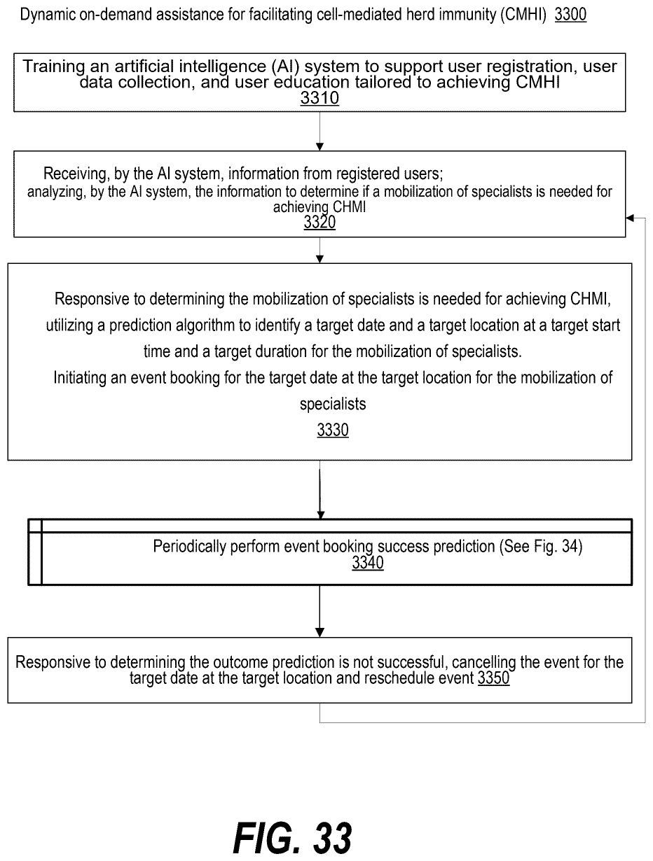

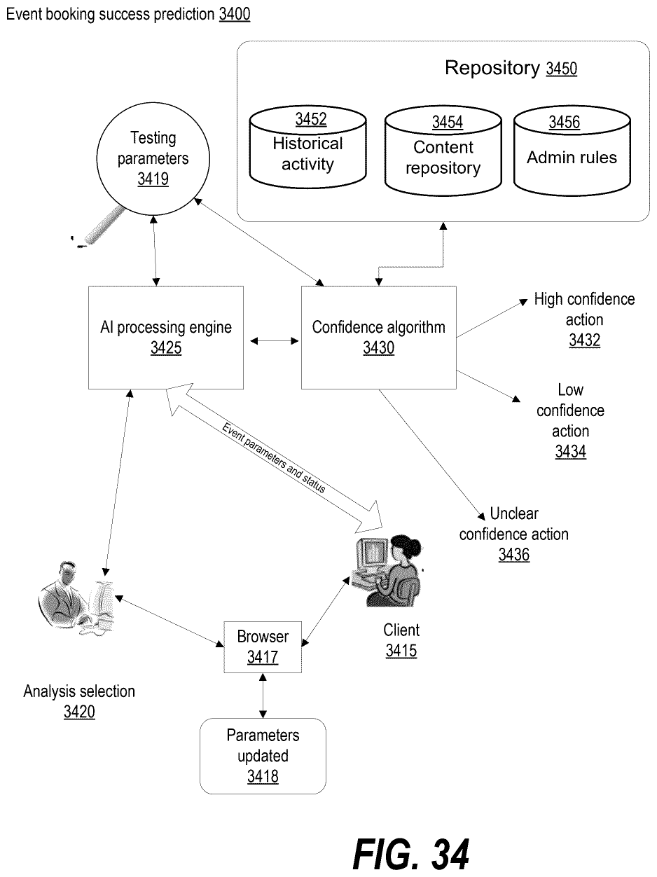

FIG. 33 shows the steps for dynamically facilitating CMHI 3300. At step 3310, the process trains an artificial intelligence (AI) system to support user registration, user data collection, and user education tailored to achieving CMHI. At step 3320, the process receives, by the AI system, information from registered users. Analyzes, by the AI system, the information to determine if a mobilization of specialists is needed for achieving CMHI. At step 3330, responsive to determining the mobilization of specialists is needed for achieving CMHI, utilizing a prediction algorithm to identify a target date and a target location at a target start time and a target duration for the mobilization of specialists. Initiates an event booking for the target date at the target location for the mobilization of specialists. At predefined process 3340, the process periodically performs event booking success prediction routine (see FIG. 34 and corresponding text for processing details). At step 3340, the process analyzes the event booking periodically to form an outcome prediction wherein the outcome prediction is one of successful and not successful. At step 3350, the process responsive to determining the outcome prediction is not successful, cancelling the event for the target date at the target location. The process loops back to step 3320.

FIG. 34 depicts an embodiment of CMHI artificial intelligence (AI) event prediction model 3400. The prediction model may be used to find matching cases to determine a statistical success rate for a successful outcome of holding a CMHI related event. The testing parameters 3419 are identified for the event. The testing parameters 3419 may include a minimum number of people to be tested, a minimum number of CMHI professionals to support the testing, and a venue booking status which may be determined by a client 3415 booking the event via any of the communication technologies. The repository 3450 may be a database management system (DBMS) supporting indexing, queries, and other typical database features. It could be any data store for recording and retrieving data. The repository 3450 may include various elements, for example, but not limited to, historical activity 3452 that records a history of events held with new events added as needed, a content repository 3454, that identifies, for example, history of previously booked events or attempts at event booking, and admin rules 3456 that may determine policies for capturing information, overriding previously entered information, and the like. The repository 3450 may have default rules for capturing factors affecting event booking. The repository 3450 may be adaptive and may automatically adjust based on feedback via artificial intelligence (AI) technology. Although the user interface depicted in FIG. 34 is browser 3417, any user interface may be used. The user interface may provide a GUI where the user inputs parameters as menu entries, command line entries, scripts entries, configuration files, .xml files, or any other means of providing the required information.

The AI processing engine 3425 uses confidence algorithm 3430 to access the repository 3450 and to characterize the CMHI parameters 3419. The analysis selection 3420 using human feedback may be tied to the confidence algorithm 3430 that formulates queries against the repository 3450 to determine comparable factors in other event processing cases. The historical activity 3452 may be retrieved as well as the information from the content repository 3454 to find associations between a current event processing case and previous event processing case histories. Natural language processing (NLP) may be applied to the historical activity 3452, to make the association. Deep analytic analysis and artificial intelligence technologies may be used to adjust the categorization. Feedback from Subject Matter Experts (SMEs), and other user feedback may be used to tune the characterization and form a confidence level or ranking to a previous event processing. Selections may be made via analysis selection 3420. Human feedback may also be used to update the event parameters 3418. The illustrative embodiment is based on a predicted improvement of the event processing case matching based on the confidence algorithm 3430. If the confidence is high that the parameters match a set of event processing, the high confidence action 3432 may add the match to the content repository 3454. In which case, statistics for the current case may be tracked and added to an existing entry. If the confidence is low, that the parameters match a previous set of event booking, a low confidence action 3434 is taken. The low confidence action 3434 may be an indication that no match was found. In that case, a new event booking case may be added to the content repository 3454. If the confidence is unclear, an unclear confidence action 3436 may be taken to request more clarification and the information may be added to the content repository 3454 as information to be gathered.

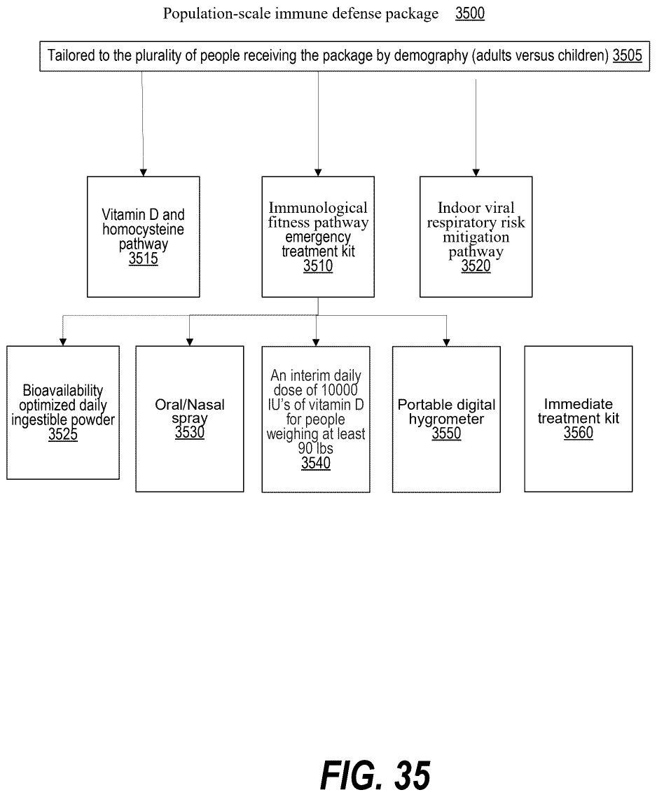

Turning now to FIG. 35, details of the population-scale immune defense package 3500 are portrayed. This is tailored to the plurality of people receiving the package by demography (adults versus children) 3505. The package comprises vitamins D and homocysteine pathway 3515, the immunological fitness pathway emergency treatment kit 3510 and the indoor viral respiratory risk mitigation pathway 3520. The immunological fitness pathway emergency treatment kit 3510 comprises: bioavailability optimized daily ingestible powder 3525, oral/Nasals spray 3530, an interim daily dose of 10000 IU's of vitamin D for people weighing at least 90 lbs 3540, portable digital hygrometer 3550, and immediate treatment kit 3560.

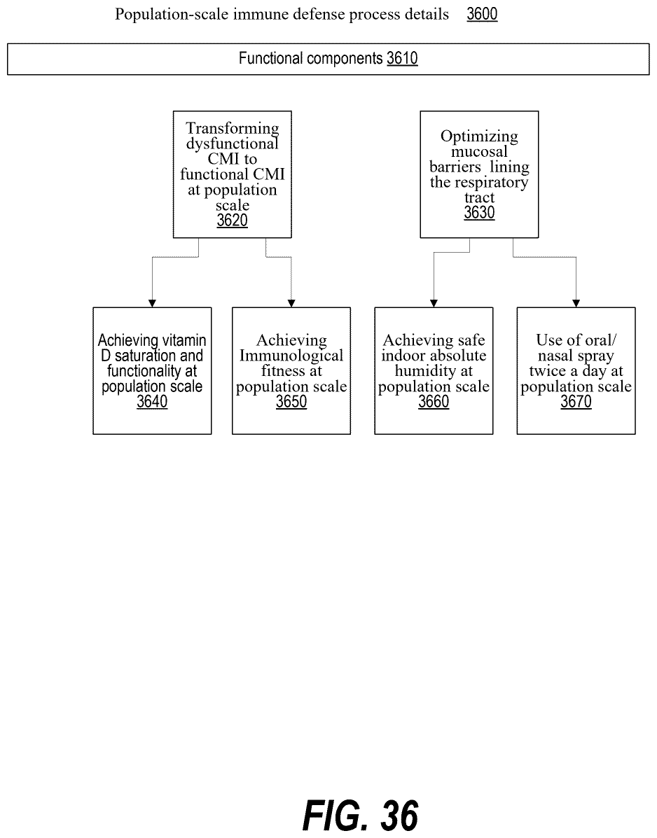

FIG. 36 depicts a functional overview of a population-scale immune defense process 3600. This is an example of population-scale immune defense package described in 106 found in FIG. 1. The functional components 3610 are: at step 3620, the process of transforming dysfunctional CMI to functional CMI at population scale includes: at step 3640 achieving vitamin D saturation and functionality at population scale and at step 3650, achieving immunological fitness at population scale. At step 3630, the process optimizes mucosal barriers lining the respiratory tract 3645 by: at step 3660 achieving safe indoor absolute humidity at population scale and at step 3670 use of oral/nasal spray twice a day at population scale.

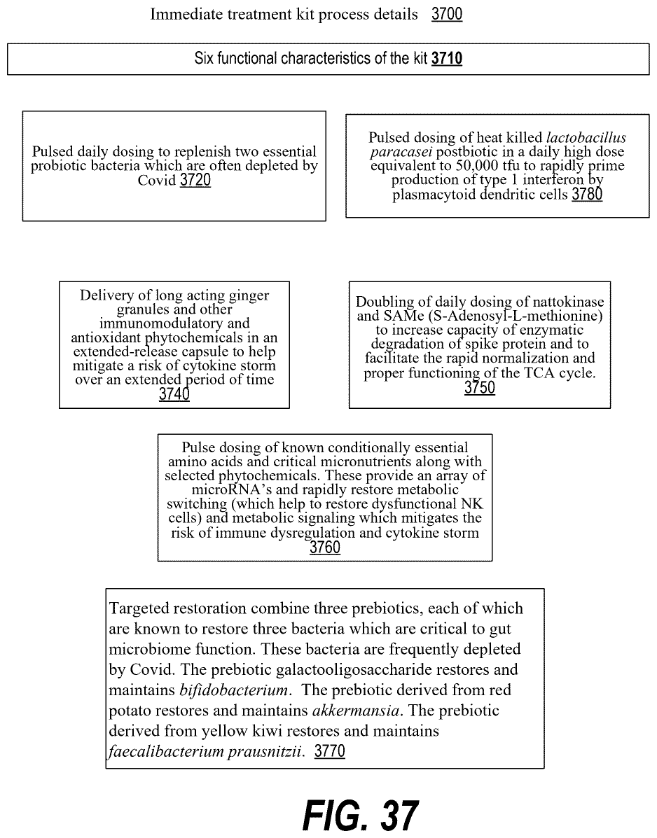

FIG. 37 depicts details of an immediate treatment kit 3700. The functional characteristics of the immediate treatment kit 3710 are: At step 3720, pulsed daily dosing to replenish two essential probiotic bacteria which are often depleted by Covid. At step 3730, pulsed dosing of heat killed lactobacillus paracasei postbiotic in a daily high dose equivalent to 50,000 tfu to rapidly prime production of type 1 interferon by plasmacytoid dendritic cells. At step 3740, the process delivers long acting ginger granules and other immunomodulatory and antioxidant phytochemicals in an extended-release capsule to help mitigate a risk of cytokine storm over an extended period of time. At step 3750, the process doubles a baseline daily dosing which varies between adults and children for: (1) nattokinase, which increase the capacity of enzymatic degradation of spike protein and (2) SAMe (S-Adenosyl-L-methionine), which facilitates the rapid normalization and proper functioning of the TCA cycle. At step 3760, the process pulse doses known conditionally essential amino acids and critical micronutrients along with selected phytochemicals. These provide an array of microRNAs and rapidly restore metabolic switching (which help to restore dysfunctional NK cells) and metabolic signaling which mitigates the risk of immune dysregulation and cytokine storm. At step 3770, the process targets restoration combine three prebiotics, each of which are known to restore three bacteria which are critical to gut microbiome function. These bacteria are frequently depleted by Covid. The prebiotic galactooligosaccharide restores and maintains bifidobacterium.The prebiotic derived from red potato restores and maintains akkermansia.The prebiotic derived from yellow kiwi restores and maintains faecalibacterium prausnitzii.

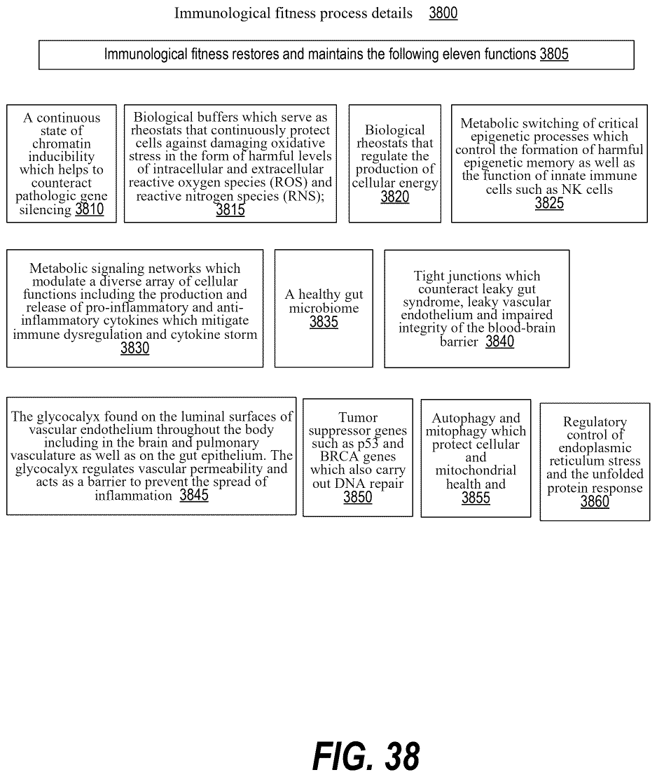

FIG. 38 depicts details of the immunological fitness process 3800. At step 3805, the immunological fitness process restores and maintains the following eleven functions: At step 3810, the process facilitates a continuous state of chromatin inducibility which helps to counteract pathologic gene silencing. At step 3815, the process restores and maintains biological buffers which serve as rheostats that continuously protect cells against damaging oxidative stress in the form of harmful levels of intracellular and extracellular reactive oxygen species (ROS) and reactive nitrogen species (RNS). At step 3820, the process restores and maintains biological rheostats that regulate the production of cellular energy. At step 3825, the process restores and maintains metabolic switching of critical epigenetic processes which controls the formation of harmful epigenetic memory as well as the function of innate immune cells such as NK cells. At step 3830, the process restores and maintains metabolic signaling networks which modulate a diverse array of cellular functions including the production and release of pro-inflammatory and anti-inflammatory cytokines which mitigate immune dysregulation and cytokine storm. At step 3835, the process restores and maintains a healthy gut microbiome. At step 3840, the process restores and maintains tight junctions which counteract leaky gut syndrome, leaky vascular endothelium and impaired integrity of the blood-brain barrier. At step 3845, the process restores and maintains the glycocalyx found on the luminal surfaces of vascular endothelium throughout the body including in the brain and pulmonary vasculature as well as on the gut epithelium. The glycocalyx regulates vascular permeability and acts as a barrier to prevent the spread of inflammation. At step 3850, the process restores and maintains tumor suppressor genes such as p53 and BRCA genes which also carry out DNA repair. At step 3855, the process restores and maintains autophagy and mitophagy which protect cellular and mitochondrial health. At step 3860, the process restores and maintains regulatory control of endoplasmic reticulum stress and the unfolded protein response.

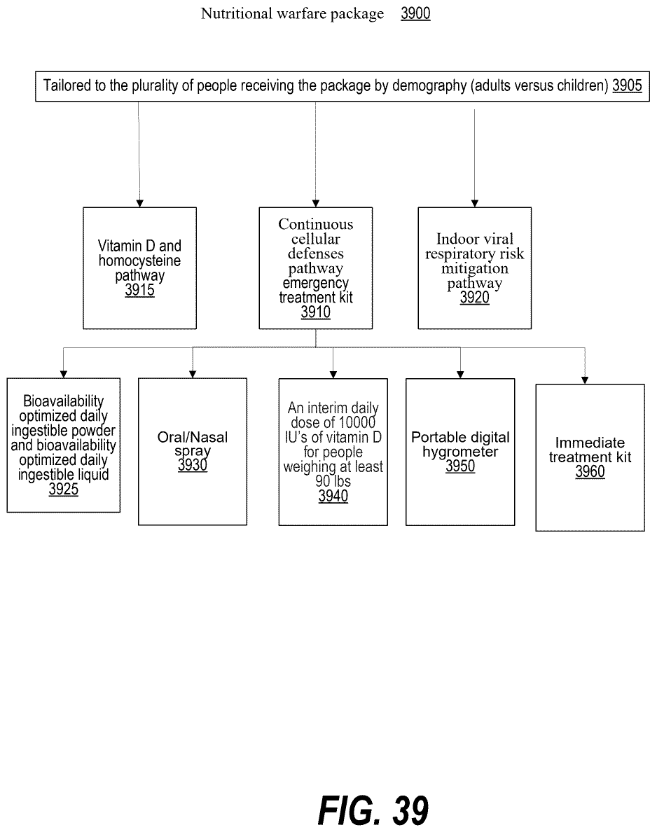

Turning now to FIG. 39, details of the nutritional warfare package 3900 are portrayed. This is tailored to the plurality of people receiving the package by demography (adults versus children) 3905. The package comprises vitamins D and homocysteine pathway 3915, the continuous cellular defenses pathway emergency treatment kit 3910 and the indoor viral respiratory risk mitigation pathway 3920. The immunological fitness pathway emergency treatment kit 3910 comprises: bioavailability optimized daily ingestible powder and bioavailability optimized daily ingestible liquid 3925, oral/nasal spray 3930, an interim daily dose of 10000 IU's of vitamin D for people weighing at least 90 lbs 3940, portable digital hygrometer 3950, and immediate treatment kit 3960.

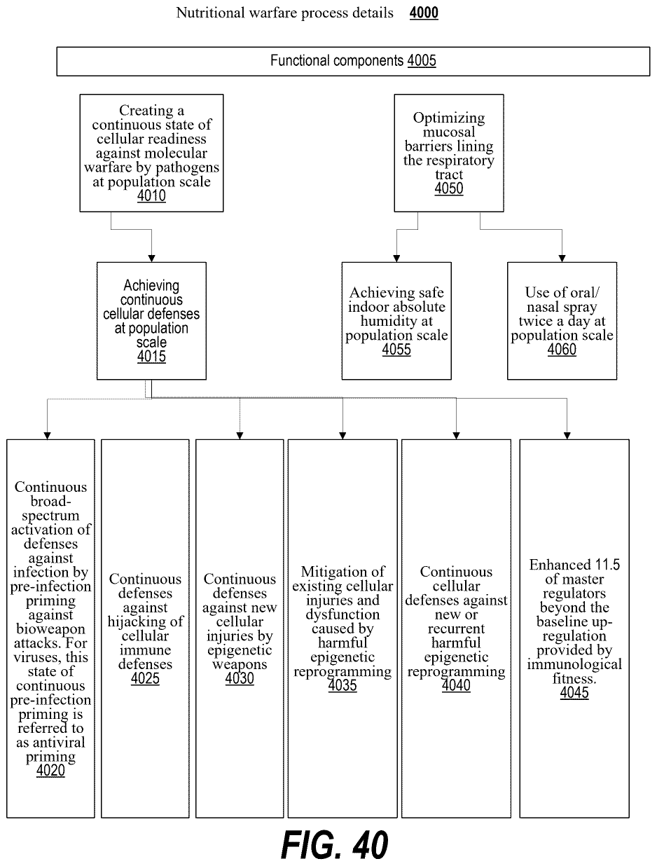

FIG. 40 depicts details of nutritional warfare process 4000. The functional components of the process 4005 are: At step 4010, the process creates a continuous state of cellular readiness against molecular warfare by pathogens at population scale. At step 4015, the process achieves continuous cellular defenses at population scale by: At step 4020, the process achieves continuous broad-spectrum activation of defenses against infection by pre-infection priming against bioweapon attacks. For viruses, this state of continuous pre-infection priming is referred to as antiviral priming. At step 4025, the process achieves continuous defenses against hijacking of cellular immune defenses. At step 4030, the process achieves continuous defenses against new cellular injuries by epigenetic weapons. At step 4035, the process achieves mitigation of existing cellular injuries and dysfunction caused by harmful epigenetic reprogramming. At step 4040, the process achieves continuous cellular defenses against new or recurrent harmful epigenetic reprogramming. At step 4045, the process achieves enhanced maintenance of master regulators beyond the baseline up-regulation provided by immunological fitness. At step 4050, the process optimizes mucosal barriers lining the respiratory tract. At step 4055, the process achieves safe indoor absolute humidity at population scale. At step 4060, the process recommends use of oral/nasal spray twice a day at population scale.

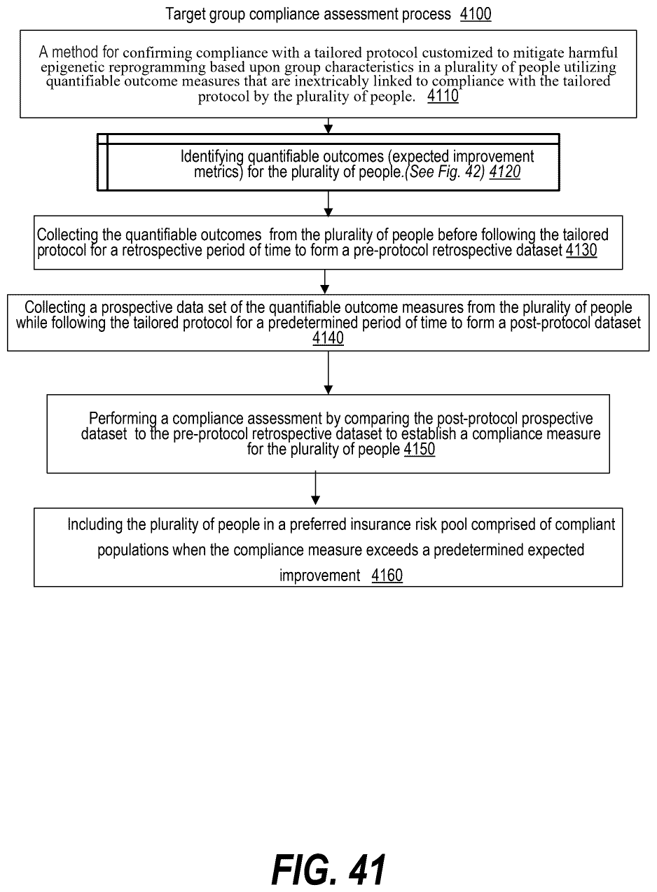

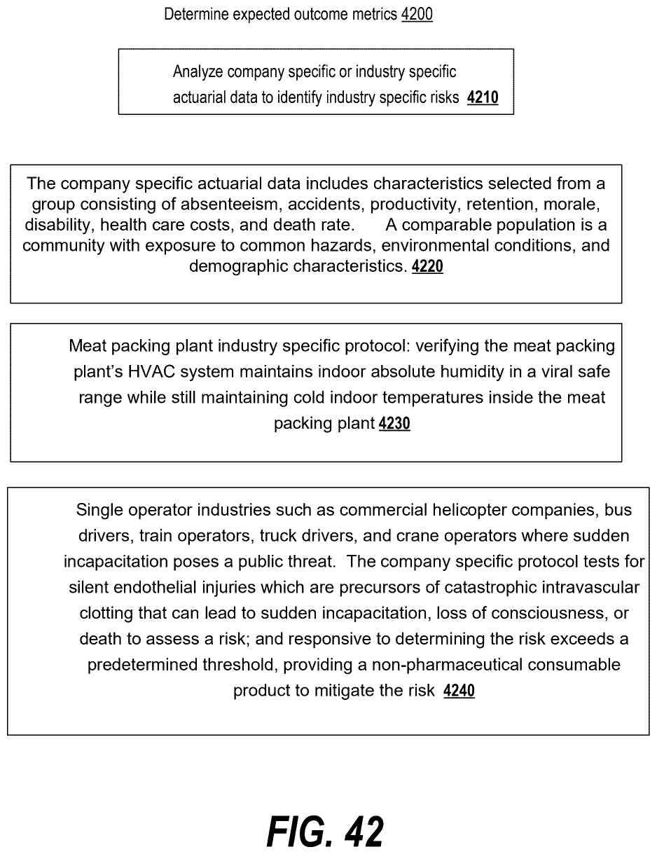

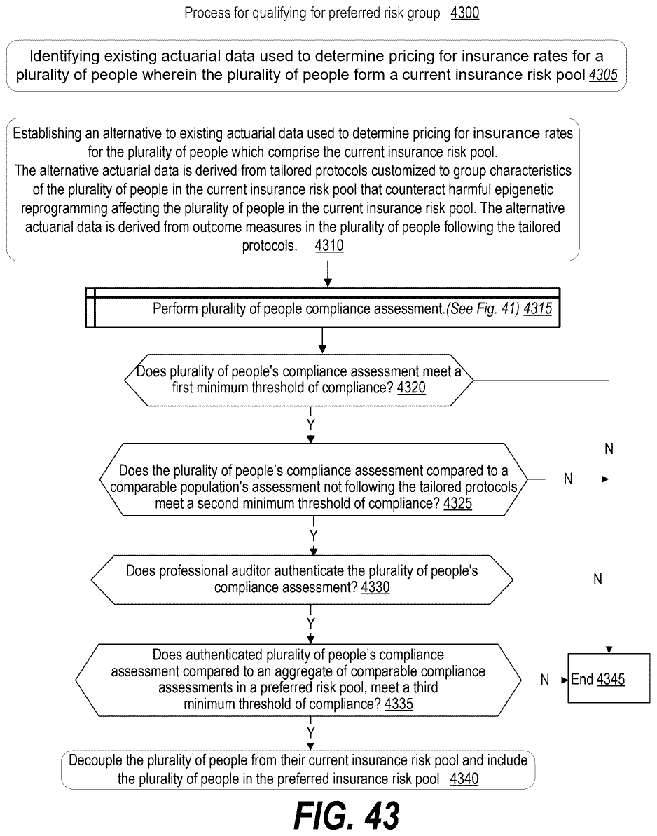

FIG. 41 depicts a target group compliance assessment process 4100. The method is an approach for confirming compliance with a tailored protocol customized to mitigate harmful epigenetic reprogramming based upon group characteristics in a plurality of people utilizing quantifiable outcome measures that are inextricably linked to compliance with the tailored protocol by the plurality of people 4110. At step 4120, the process identifies quantifiable outcome measures (i.e., expected improvement metrics) for the plurality of people. (See FIG. 42). At step 4130, the process collects the quantifiable outcomes from the plurality of people before following the tailored protocol for a retrospective period of time to form a pre-protocol retrospective dataset. At step 4140, the process collects a prospective data set of the quantifiable outcome measures from the plurality of people while following the tailored protocol for a predetermined period of time to form a post-protocol dataset. At step 4150, the process performs a compliance assessment by comparing the post-protocol prospective dataset to the pre-protocol retrospective dataset to establish a compliance measure for the plurality of people. At step 4160, the process includes the plurality of people in a preferred insurance risk pool comprised of compliant populations when the compliance measure exceeds a predetermined expected improvement.

FIG. 42 depicts a process of determining expected outcome metrics 4200. At step 4210, the process analyzes company specific data or industry specific actuarial data to identify industry specific risks. At step 4220, the process compares company specific actuarial data to others with similar characteristics, that is, a comparable population such as, a community with exposure to common hazards, environmental conditions, and demographic characteristics. includes characteristics selected from a group consisting of absenteeism, accidents, productivity, retention, morale, disability, health care costs, and death rate. At step 4230, the process utilizes an example of a meat packing plant industry specific protocol which verifies the meat packing plant's HVAC system maintains indoor absolute humidity in a viral safe range while still maintaining cold indoor temperatures inside the meat packing plant. At step 4240, the process references single operator industries such as commercial helicopter companies, bus drivers, train operators, truck drivers, and crane operators where sudden incapacitation poses a public threat. The company specific protocol tests for silent endothelial injuries which are precursors of catastrophic intravascular clotting that can lead to sudden incapacitation, loss of consciousness, or death to assess a risk; and responsive to determining the risk exceeds a predetermined threshold, providing a non-pharmaceutical consumable product to mitigate the risk.

FIG. 43 depicts a process for qualifying for a preferred insurance risk pool 4300. At step 4305, the process identifies existing actuarial data used to determine pricing for insurance rates for a plurality of people wherein the plurality of people form a current insurance risk pool. At step 4310, the process establishes an alternative to existing actuarial data used to determine pricing for insurance rates for the plurality of people which comprise the current insurance risk pool. The alternative actuarial data is derived from tailored protocols customized to group characteristics of the plurality of people in the current insurance risk pool that counteract harmful epigenetic reprogramming affecting the plurality of people in the current insurance risk pool. The alternative actuarial data is derived from outcome measures in the plurality of people following the tailored protocols. At the predefined process 4315, the process performs the target group compliance assessment process (see FIG. 41 and corresponding text for processing details). If the plurality of people's compliance assessment meets a first minimum threshold of compliance (decision 4320) the process continues to step 4325 where the process continues by determining if the plurality of people's compliance assessment, when compared to a comparable population not following the tailored protocols, meet a second minimum threshold of compliance, then the process continues to step 4330 to determine whether a professional auditor authenticates the plurality of people's compliance assessment. If the professional auditor authenticates the plurality of people's compliance assessment, then the process proceeds to 4335 to determine whether the plurality of people's authenticated compliance assessment, when compared to an aggregate of comparable compliance assessments in a preferred insurance risk pool, meets a third minimum threshold of compliance. If the plurality of people's authenticated compliance assessment meets a third minimum threshold of compliance, then the process proceeds to 4340, where the process decouples the plurality of people from the current insurance risk pool and includes the plurality of people in the preferred insurance risk pool. If any of the foregoing steps fail, the process ends at step 4345.

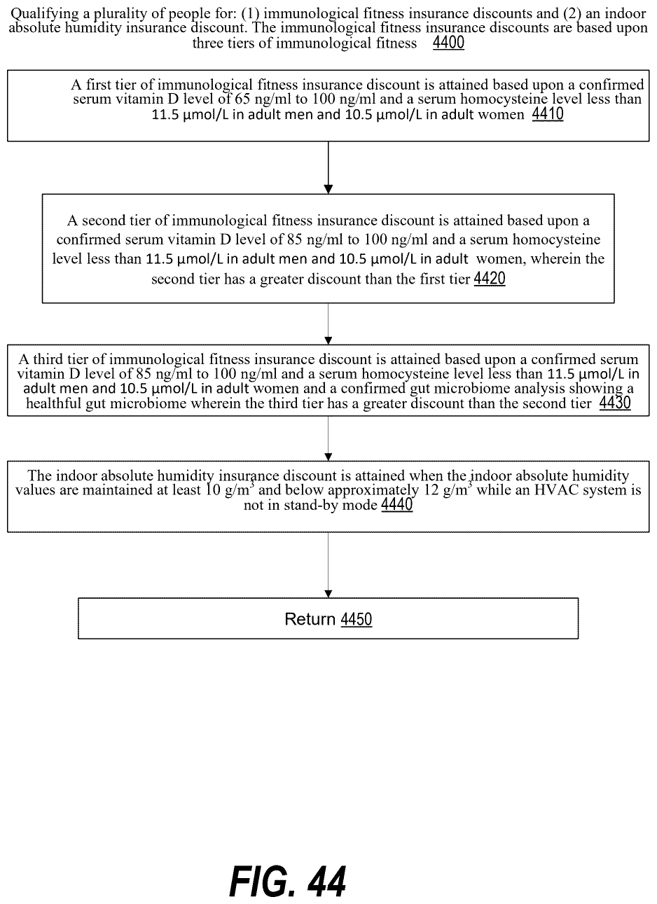

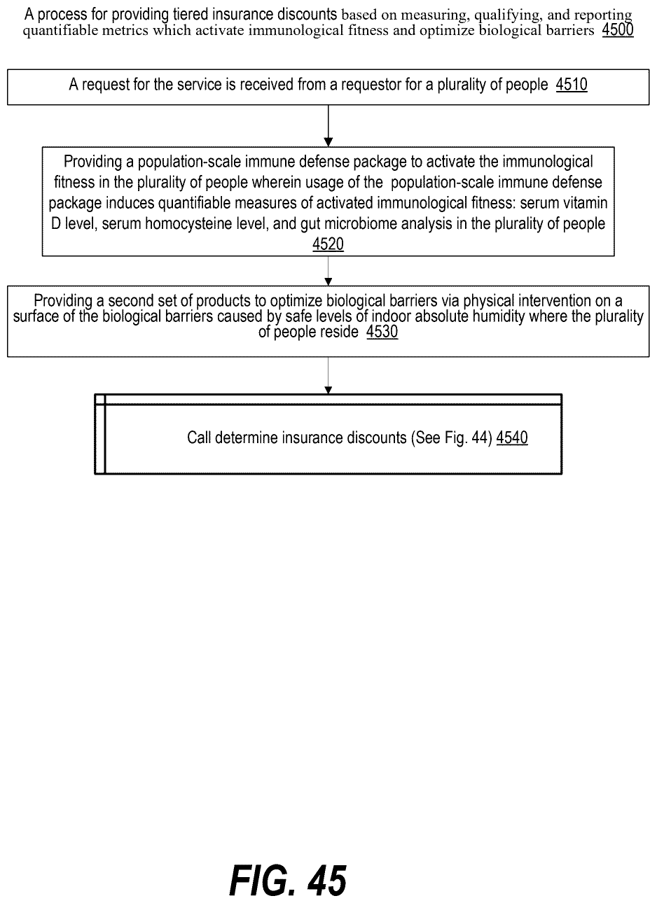

FIG. 44 depicts a process for qualifying a plurality of recipients for: (1) immunological fitness insurance discounts and (2) an indoor absolute humidity insurance discount. The immunological fitness insurance discounts are based upon three tiers of immunological fitness 4400. At step 4410, a first tier of immunological fitness insurance discount is attained based upon a confirmed serum vitamin D level of 65 ng/mL to 100 ng/mL and a serum homocysteine level less than 11.5 μmol/L in adult men and 10.5 μmol/L in adult women. At step 4420, a second tier of immunological fitness insurance discount is attained based upon a confirmed serum vitamin D level of 85 ng/mL to 100 ng/mL and a serum homocysteine level less than 11.5 μmol/L in adult men and 10.5 μmol/L in adult women, wherein the second tier has a greater discount than the first tier. At step 4430, a third tier of immunological fitness insurance discount is attained based upon a confirmed serum vitamin D level of 85 ng/mL to 100 ng/mL and a serum homocysteine level less than 11.5 μmol/L in adult men and 10.5 μmol/L in adult women and a confirmed gut microbiome analysis showing a healthful gut microbiome wherein the third tier has a greater discount than the second tier. At step 4440, the indoor absolute humidity insurance discount is attained when the indoor absolute humidity values are maintained at least 10 g/m³ and below approximately 12 g/m³ while an HVAC system is not in standby mode. FIG. 44 processing thereafter returns to the calling routine (see FIG. 45) at 4450.

FIG. 45 depicts a flow for a service for providing tiered insurance discounts based on measuring, qualifying, and reporting quantifiable metrics which activate immunological fitness and optimize biological barriers 4500. At step 4510, a request for the service is received from a requestor for a plurality of recipients. At step 4520, a population-scale immune defense package is provided to activate the immunological fitness in the plurality of recipients wherein usage of the population-scale immune defense package induces quantifiable measures of activated immunological fitness: serum vitamin D level, serum homocysteine level, and gut microbiome analysis in the plurality of recipients. At step 4530 a second set of products is provided to optimize biological barriers via physical intervention on a surface of the biological barriers caused by safe levels of indoor absolute humidity where the plurality of recipients reside. At predefined process 4540, the process performs the call determine insurance discounts routine (see FIG. 44 and corresponding text for processing details).

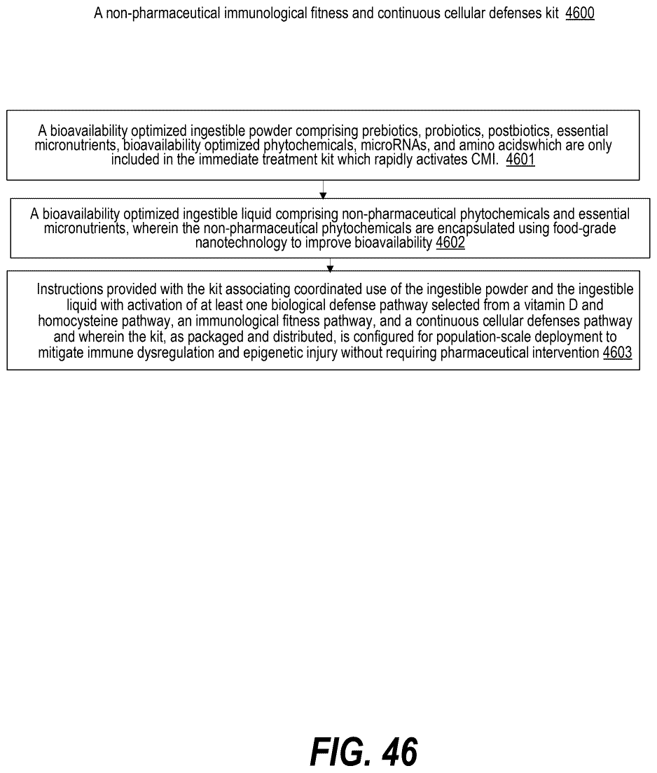

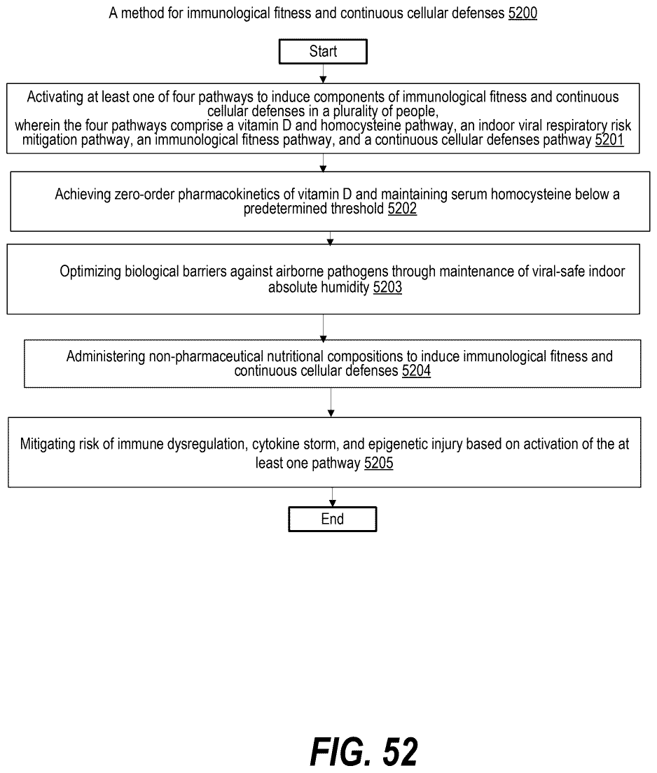

FIG. 46 depicts a non-pharmaceutical immunological fitness and continuous cellular defenses kit such as used in FIG. 1 and in FIG. 52 according to an embodiment of the invention (entry 4600). The kit comprises a bioavailability optimized ingestible powder comprising prebiotics, probiotics, postbiotics, essential micronutrients, bioavailability optimized phytochemicals, microRNAs, and amino acids (entry 4601); a bioavailability optimized ingestible liquid comprising non-pharmaceutical phytochemicals and essential micronutrients, wherein the non-pharmaceutical phytochemicals are encapsulated using food-grade nanotechnology to improve bioavailability (entry 4602); and instructions provided with the kit associating coordinated use of the ingestible powder and the ingestible liquid with activation of at least one biological defense pathway selected from a vitamin D and homocysteine pathway, an immunological fitness pathway, and a continuous cellular defenses pathway and wherein the kit, as packaged and distributed, is configured for population-scale deployment to mitigate immune dysregulation and epigenetic injury without requiring pharmaceutical intervention (entry 4603).

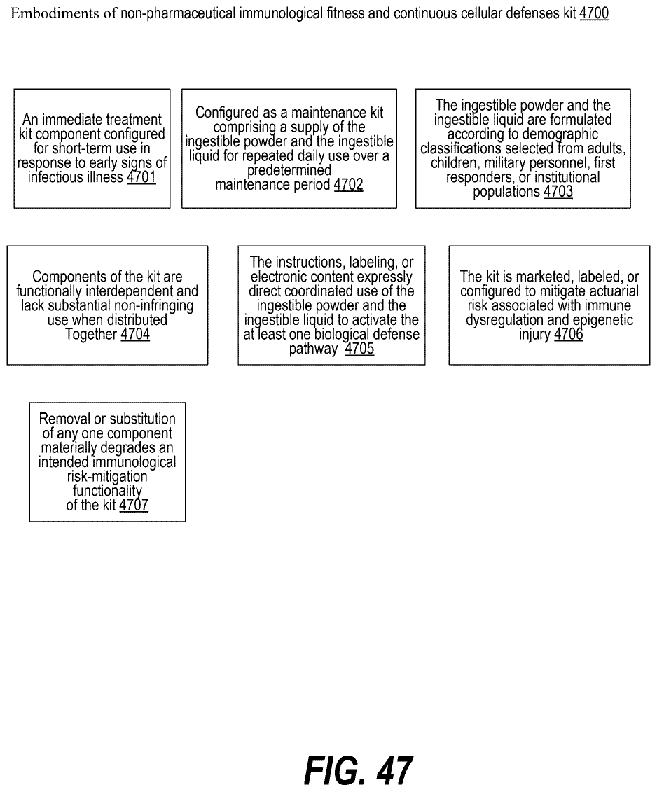

FIG. 47 depicts embodiments of a non-pharmaceutical immunological fitness and continuous cellular defenses kit (entry 4700). An immediate treatment kit component may be configured for short-term use in response to early signs of infectious illness such as used in FIG. 19 (entry 4701). The kit may be configured as a maintenance kit such as referenced at step 106 in FIG. 1, comprising a supply of the ingestible powder and the ingestible liquid for repeated daily use over a predetermined maintenance period (entry 4702). The ingestible powder and the ingestible liquid may be formulated according to demographic classifications such as, referenced in FIG. 35 selected from adults, children, military personnel, first responders, or institutional populations (entry 4703). The components of the kit may be functionally interdependent and lack substantial non-infringing use when distributed together. populations (entry 4704). The instructions, labeling, or electronic content may expressly direct coordinated use of the ingestible powder and the ingestible liquid to activate the at least one biological defense pathway (entry 4705). The kit may be marketed, labeled, or configured to mitigate actuarial risk associated with immune dysregulation and epigenetic injury (entry 4706) Removal or substitution of any one component materially may degrades an intended immunological risk-mitigation functionality of the kit (entry 4707).

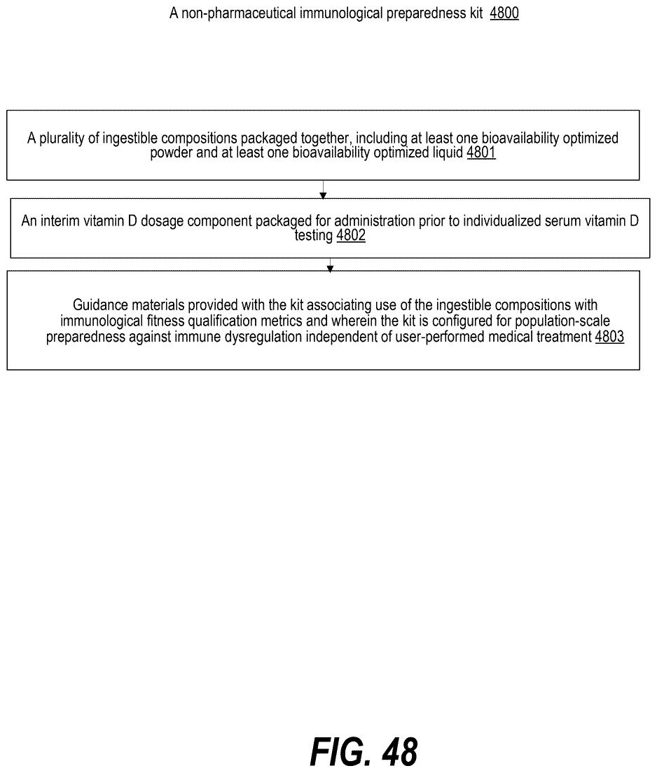

FIG. 48 depicts a non-pharmaceutical immunological preparedness kit according to an embodiment of the invention (entry 4800). The non-pharmaceutical immunological preparedness kit comprises a plurality of ingestible compositions packaged together, including at least one bioavailability optimized powder and at least one bioavailability optimized liquid (entry 4801); an interim vitamin D dosage component packaged for administration prior to individualized serum vitamin D testing (entry 4802); and guidance materials provided with the kit associating use of the ingestible compositions with immunological fitness qualification metrics and wherein the kit is configured for population-scale preparedness against immune dysregulation independent of user-performed medical treatment (entry 4803).

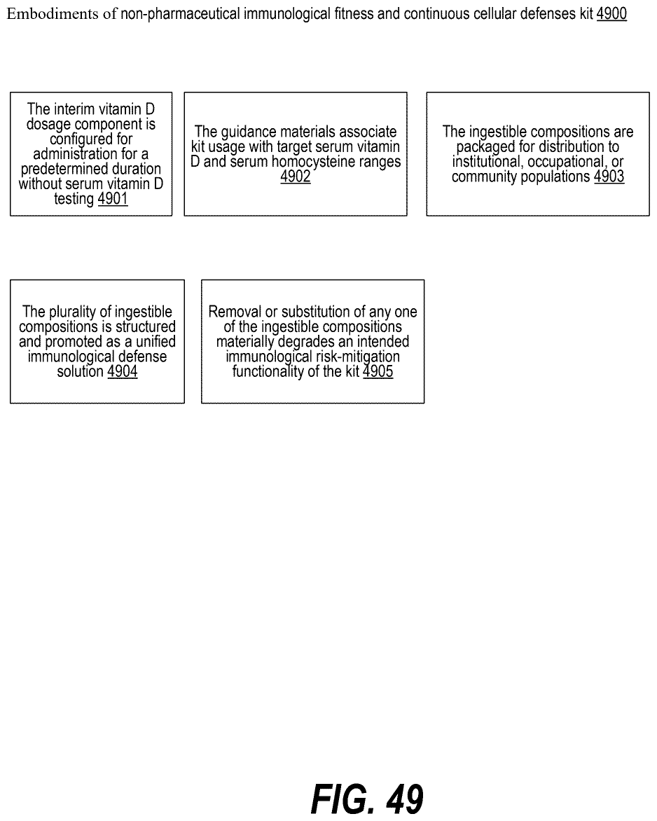

FIG. 49 depicts embodiments of non-pharmaceutical immunological fitness and continuous cellular defenses kit (entry 4900). The interim vitamin D dosage component may be configured for administration for a predetermined duration without serum vitamin D testing (entry 4901). The guidance materials may associate kit usage with target serum vitamin D and serum homocysteine ranges (entry 4902). The ingestible compositions may be packaged for distribution to institutional, occupational, or community populations (entry 4903). The plurality of ingestible compositions may be structured and promoted as a unified immunological defense solution (entry 4904). Removal or substitution of any one of the ingestible compositions materially may degrades an intended immunological risk-mitigation functionality of the kit (entry 4905).

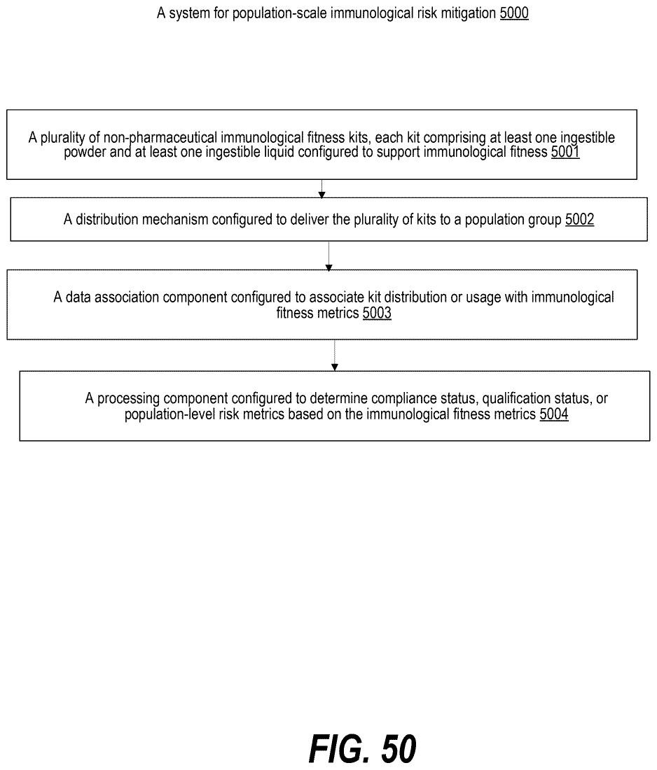

FIG. 50 depicts a system for population-scale immunological risk mitigation (entry 5000). The system comprises a plurality of non-pharmaceutical immunological fitness kits, each kit comprising at least one ingestible powder and at least one ingestible liquid configured to support immunological fitness (entry 5001); a distribution mechanism configured to deliver the plurality of kits to a population group (entry 5002); a data association component configured to associate kit distribution or usage with immunological fitness metrics (entry 5003); and a processing component configured to determine compliance status, qualification status, or population-level risk metrics based on the immunological fitness metrics (entry 5004).

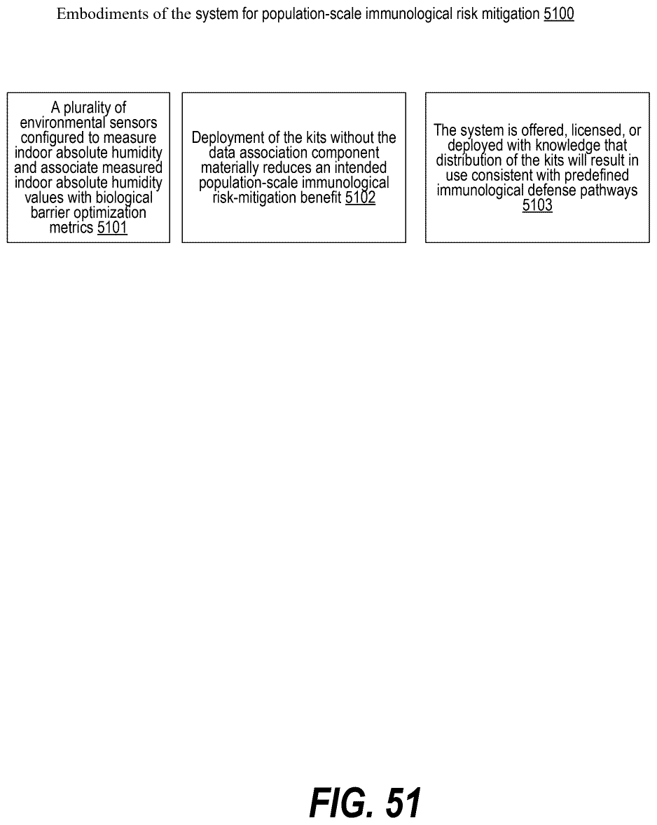

FIG. 51 depicts embodiments of the system for population-scale immunological risk mitigation (entry 5100). The system may comprise a plurality of environmental sensors configured to measure indoor absolute humidity and associate measured indoor absolute humidity values with biological barrier optimization metrics (entry 5101). Deployment of the kits without the data association component may materially reduce an intended population-scale immunological risk-mitigation benefit (entry 5102). The system may be offered, licensed, or deployed with knowledge that distribution of the kits will result in use consistent with predefined immunological defense pathways (entry 5103).

FIG. 52 depicts flow diagram of a method for immunological fitness and continuous cellular defenses as shown in FIG. 1 according to an illustrative embodiment (step 5200). The method activates at least one of four pathways to induce components of immunological fitness and continuous cellular defenses in a plurality of people. The four pathways comprise a vitamin D and homocysteine pathway, an indoor viral respiratory risk mitigation pathway, an immunological fitness pathway, and a continuous cellular defenses pathway (step 5201). The method achieves zero-order pharmacokinetics of vitamin D and maintaining serum homocysteine below a predetermined threshold (step 5202). The method optimizes biological barriers against airborne pathogens through maintenance of viral-safe indoor absolute humidity (step 5203). The method administers non-pharmaceutical nutritional compositions to induce immunological fitness and continuous cellular defenses (step 5204). The method mitigates risk of immune dysregulation, cytokine storm, and epigenetic injury based on activation of the at least one pathway (step 5205). The method thereafter ends.

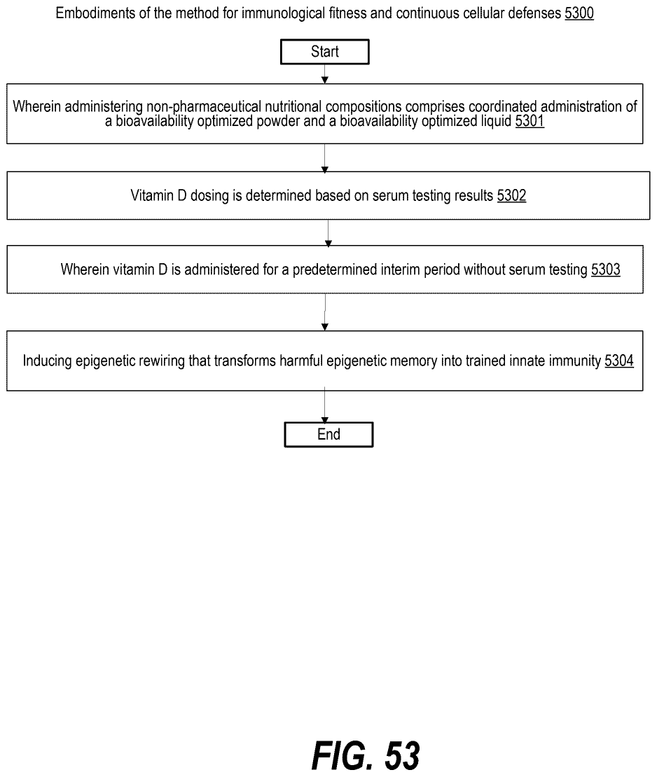

FIG. 53 depicts embodiments of the method for immunological fitness and continuous cellular defenses shown in FIG. 52 (step 5300). The administered non-pharmaceutical nutritional compositions comprises coordinated administration of a bioavailability optimized powder and a bioavailability optimized liquid (step 5301). The method may determine vitamin D dosing based on serum testing results (step 5302). The method may administer vitamin D for a predetermined interim period without serum testing (step 5303). The method may induce epigenetic rewiring that transforms harmful epigenetic memory into trained innate immunity (step 5304). The method thereafter end.

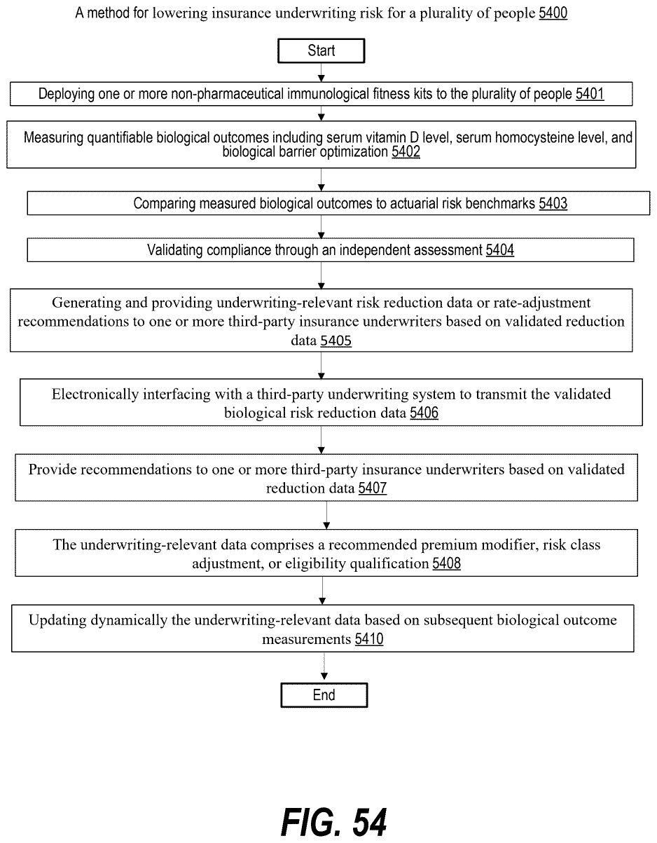

FIG. 54 depicts a flow diagram of a method for lowering insurance underwriting risk for a plurality of people according to an illustrative embodiment (step 5400). The method deploys one or more non-pharmaceutical immunological fitness kits to the plurality of people (step 5401). The method measures quantifiable biological outcomes including serum vitamin D level, serum homocysteine level, and biological barrier optimization (step 5402). The process compares measured biological outcomes to actuarial risk benchmarks (step 5403). The process validates compliance through an independent assessment (step 5404). The process generates and provides underwriting-relevant risk reduction data or rate-adjustment recommendations to one or more third-party insurance underwriters based on validated reduction data (step 5405). The underwriting-relevant data may include a recommended premium modifier, risk class adjustment, or eligibility qualification. The process forms a preferred insurance risk pool comprising populations demonstrating sustained compliance with immunological fitness protocols. The process assigns tiered insurance discounts based on graduated levels of immunological fitness. The process may interface electronically with a third-party-party underwriting system to transmit the validated biological risk reduction data (step 5406). The process may provide recommendations to one or more third-party insurance underwriters based on validated reduction data (step 5407). The underwriting-relevant data may include a recommended premium modifier, risk class adjustment, or eligibility qualification (step 5408). The process may update dynamically the underwriting-relevant data based on subsequent biological outcome measurements. (step 5410). The process thereafter ends.

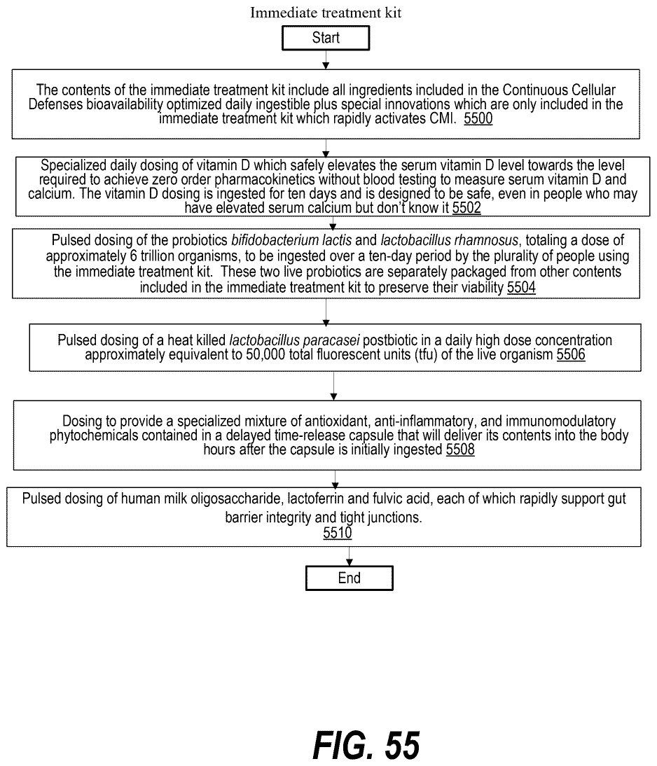

FIG. 55 depicts contents of an immediate treatment kit which includes all ingredients included in the Continuous Cellular Defenses bioavailability optimized daily ingestible plus special innovations which are only included in the immediate treatment kit which rapidly activate CMI 5500.

At step 5502, the contents of the immediate treatment kit include: specialized daily dosing of vitamin D which rapidly and safely elevates the serum vitamin D level towards the approximate range of serum vitamin D required to achieve zero-order pharmacokinetics without blood testing of serum vitamin D and calcium. The specialized vitamin D dosing of approximately 30,000 IU's per day is taken for ten days. It would require a much longer period of daily dosing at this level of vitamin D intake to cause elevated serum calcium. Elevated serum calcium (hypercalcemia) is the only significant toxicity related to vitamin D ingestion.

At step 5504, the process utilizes pulsed dosing of the probiotics bifidobacterium lactis and Lactobacillus rhamnosus totaling a dose of approximately 6 trillion organisms to be ingested over a ten-day period by the plurality of people using the immediate treatment kit. These two live probiotics are separately packaged from other contents included in the immediate treatment kit to preserve their viability.

At step 5506, the process utilizes pulsed dosing of a heat killed lactobacillus paracasei postbiotic in a daily high dose concentration approximately equivalent to 50,000 total fluorescent units (tfu) of the live organism.

At step 5508, delayed dosing of a specialized mixture of antioxidant, anti-inflammatory, and immunomodulatory phytochemicals contained in a delayed time-release capsule that will deliver its contents into the body hours after the capsule is initially ingested.

At step 5510, the process utilizes pulsed dosing of human milk oligosaccharide, lactoferrin and fulvic acid, each of which rapidly support gut barrier integrity and tight junctions.

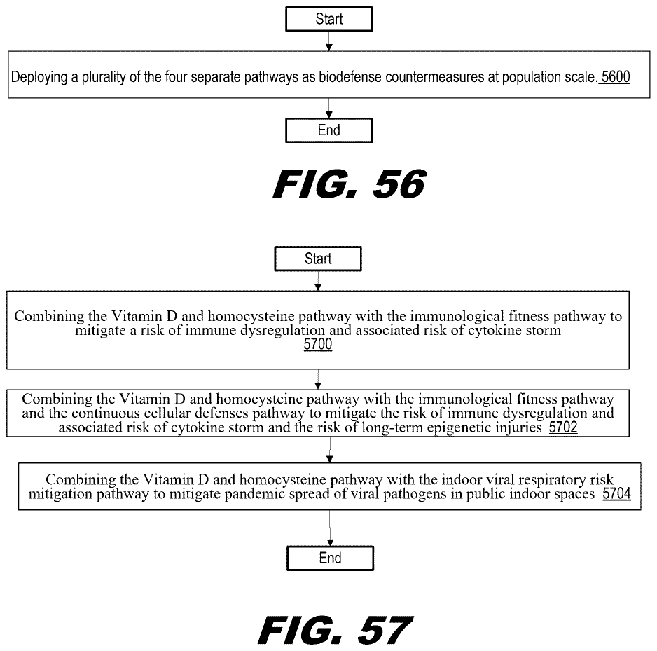

Turning now to FIG. 56, an illustrative embodiment of the process deploys a plurality of the four separate pathways as biodefense countermeasures at population scale (step 5600). In this illustrative example, any of the pathways or combination of the pathways shown in FIG. 1 can be examples of the biodefense pathways identified in step 100 in FIG. 1. The process thereafter ends.

Turning now to FIG. 57, an illustrative embodiment of the process combines the vitamin D and homocysteine pathway with the immunological fitness pathway and the continuous cellular defenses pathway to mitigate a risk of immune dysregulation and associated risk of cytokine storm (step 5700). The process combines the Vitamin D and homocysteine pathway with the immunological fitness pathway and the continuous cellular defenses pathway to mitigate the risk of immune dysregulation and associated risk of cytokine storm and the risk of long-term epigenetic injuries (step 5702). The process combines the Vitamin D and homocysteine pathway with the indoor viral respiratory risk mitigation pathway to mitigate pandemic spread of viral pathogens in public indoor spaces (step 5704). These are illustrative combinations of the biodefense pathways identified in step 100 in FIG. 1. The process thereafter ends.

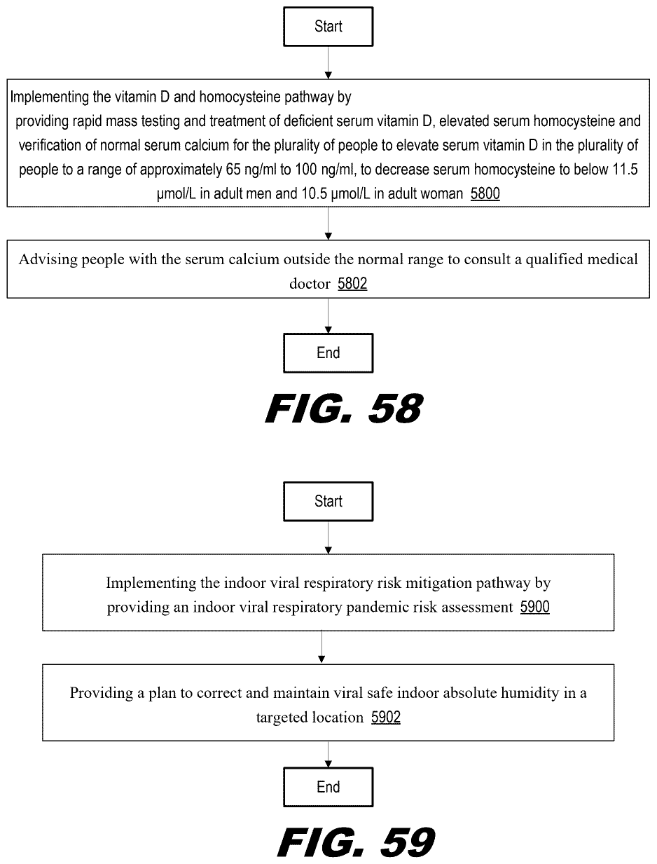

Turning now to FIG. 58, an illustrative embodiment of the process implements the vitamin D and homocysteine pathway by providing rapid mass testing and treatment of deficient serum vitamin D, elevated serum homocysteine and verification of normal serum calcium for the plurality of people to elevate serum vitamin D in the plurality of people to a range of approximately 65 ng/mL to 100 ng/mL, to decrease serum homocysteine to below 11.5 μmol/L in adult men and 10.5 μmol/L in adult woman 5800. The process advises people with a serum calcium outside the normal range to consult a qualified medical doctor 5802. These are illustrative combinations of the biodefense pathways identified in step 102 in FIG. 1. The process thereafter ends.

Turning now to FIG. 59, an illustrative embodiment of the process that implements the indoor viral respiratory risk mitigation pathway by providing an indoor viral respiratory pandemic risk assessment 5900. The process provides a plan to correct and maintain viral safe indoor absolute humidity in a targeted location 5902. These are illustrative combinations of the biodefense pathways identified in step 104 in FIG. 1. The process thereafter ends.

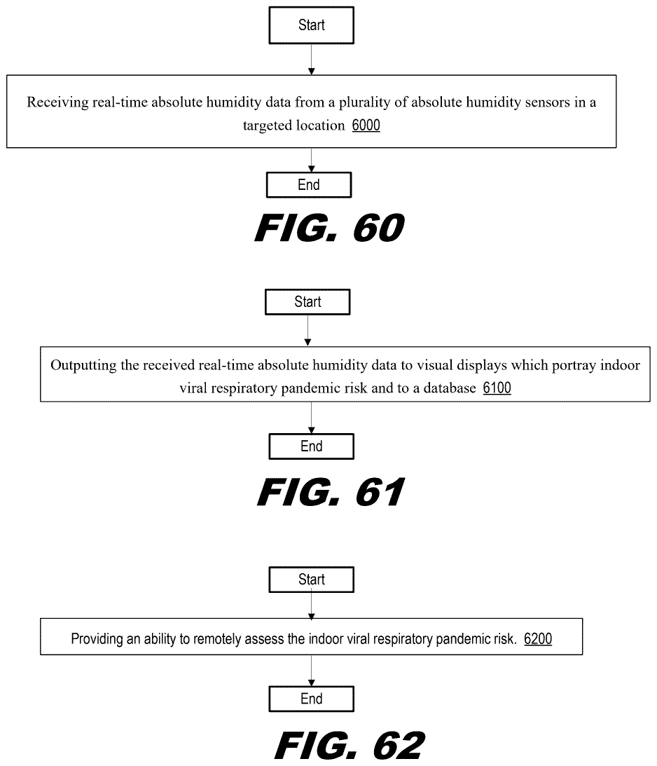

Turning now to FIG. 60, an illustrative embodiment of the process that receives real-time absolute humidity data from a plurality of absolute humidity sensors in a targeted location 6000. These are illustrative combinations of the real-time data received in step 410 in FIG. 4. The process thereafter ends.

Turning now to FIG. 61, an illustrative embodiment of the process that outputs the received real-time absolute humidity data to visual displays which portray indoor viral respiratory pandemic risk and to a database 6100. The output to the visual display is an illustrative example of step 630 in FIG. 6. In addition, these are illustrative combinations of the storing data in step 440 and transferring data in step 470 in FIG. 4. The process thereafter ends.

Turning now to FIG. 62, an illustrative embodiment provides the ability to remotely assess the indoor viral respiratory pandemic risk information in 6200. These are illustrative combinations of the storing data in step 440 and transferring data in step 675 in FIG. 6. The process thereafter ends.

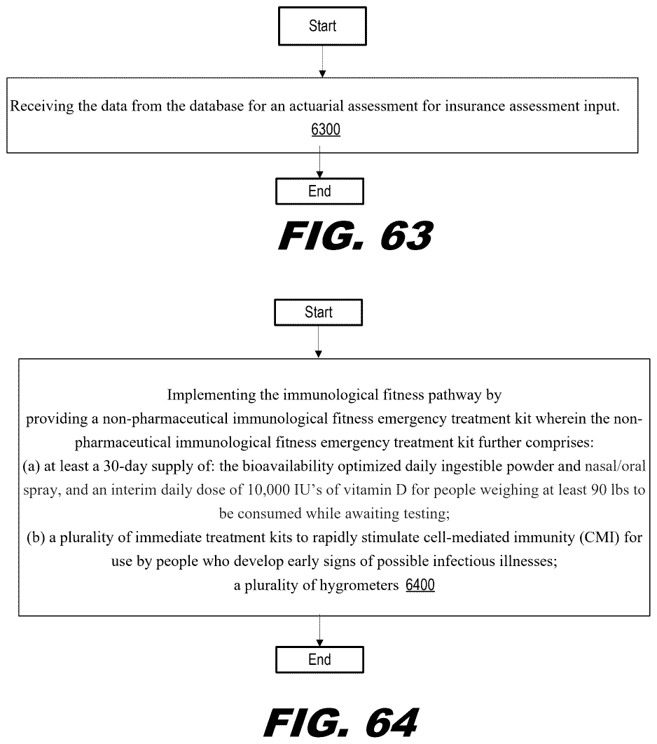

Turning now to FIG. 63, an illustrative embodiment of receiving the data from the database for an actuarial assessment for insurance assessment input 6300. These are illustrative embodiments of utilizing APIs as shown in step 1100 in FIG. 11. This is an example of how to retrieve data for the insurance assessment input such as those found in step 7804 in FIG. 78. The process thereafter ends.

Turning now to FIG. 64, an illustrative embodiment of implementing the immunological fitness pathway by providing a non-pharmaceutical immunological fitness emergency treatment kit wherein the non-pharmaceutical immunological fitness emergency treatment kit includes: providing a non-pharmaceutical immunological fitness emergency treatment kit wherein the non-pharmaceutical immunological fitness emergency treatment kit further comprises: (a) at least a 30-day supply of: the bioavailability optimized daily ingestible powder and nasal/oral spray, and an interim daily dose of 10,000 IU's of vitamin D for people weighing at least 90 lbs to be consumed while awaiting testing; (b) a plurality of immediate treatment kits to rapidly stimulate cell-mediated immunity (CMI) for use by people who develop early signs of possible infectious illnesses; and a plurality of hygrometers 6400. These are illustrative examples of the activating the immunological fitness pathway in step 106 in FIG. 1. The process thereafter ends.

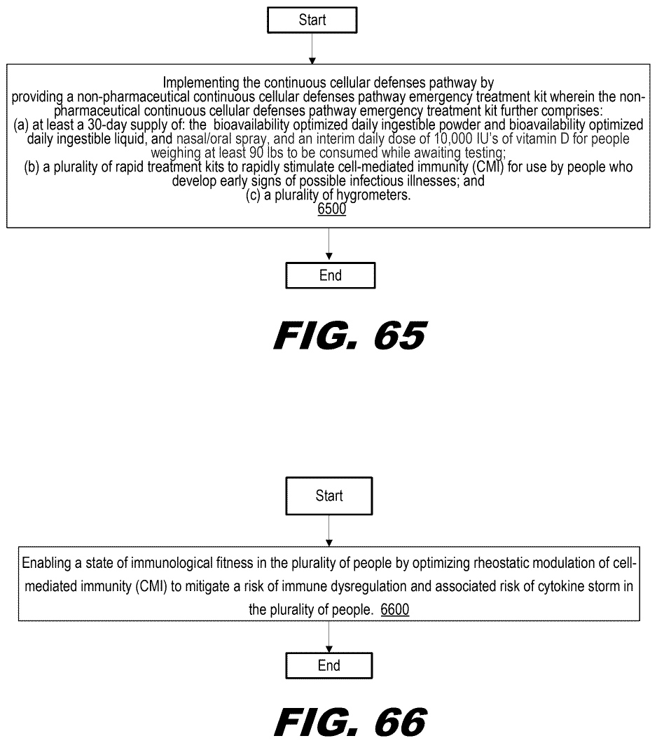

Turning now to FIG. 65, an illustrative embodiment of implementing the continuous cellular defenses pathway by providing a non-pharmaceutical continuous cellular defenses pathway emergency treatment kit wherein the non-pharmaceutical continuous cellular defenses pathway emergency treatment kit further comprises: (a) at least a 30-day supply of: the bioavailability optimized daily ingestible powder and bioavailability optimized daily ingestible liquid, and nasal/oral spray, and an interim daily dose of 10,000 IU's of vitamin D for people weighing at least 90 lbs to be consumed while awaiting testing; (b) a plurality of rapid treatment kits to rapidly stimulate cell-mediated immunity (CMI) for use by people who develop early signs of possible infectious illnesses; and (c) a plurality of hygrometers 6500. These are illustrative examples of the activating the continuous cellular defenses pathway in step 110 in FIG. 1. The process thereafter ends.

Turning now to FIG. 66, an illustrative embodiment of enabling a state of immunological fitness in the plurality of people by optimizing rheostatic modulation of cell-mediated immunity (CMI) to mitigate a risk of immune dysregulation and associated risk of cytokine storm in the plurality of people 6600. These are illustrative examples of enabling a state immunological fitness in step 106 in FIG. 1. The process thereafter ends.

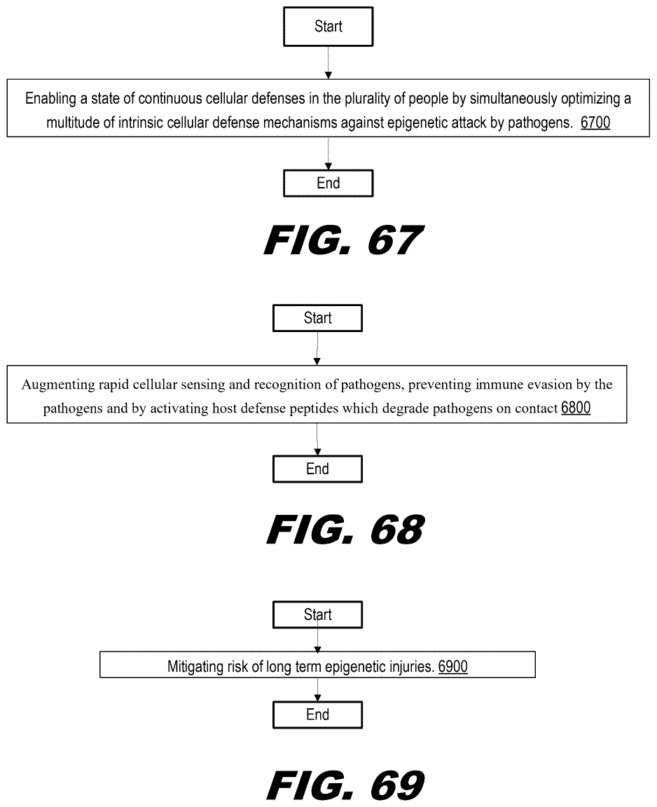

FIG. 67 depicts an illustrative embodiment of enabling a state of continuous cellular defenses in the plurality of people by simultaneously optimizing a multitude of intrinsic cellular defense mechanisms against epigenetic attack by pathogens 6700. These are illustrative examples of enabling a state continuous cellular defenses in step 110 in FIG. 1. The process thereafter ends.

FIG. 68 depicts an illustrative embodiment of augmenting rapid cellular sensing and recognition of pathogens, preventing immune evasion by the pathogens and by activating host defense peptides which degrade pathogens on contact 6800. These are illustrative examples of enabling a state of continuous cellular defenses in step 110 in FIG. 1. The process thereafter ends.

FIG. 69 depicts a process of mitigating risk of long term epigenetic injuries 6900. These are illustrative examples of enabling a state of continuous cellular defenses in step 110 in FIG. 1. The process thereafter ends.

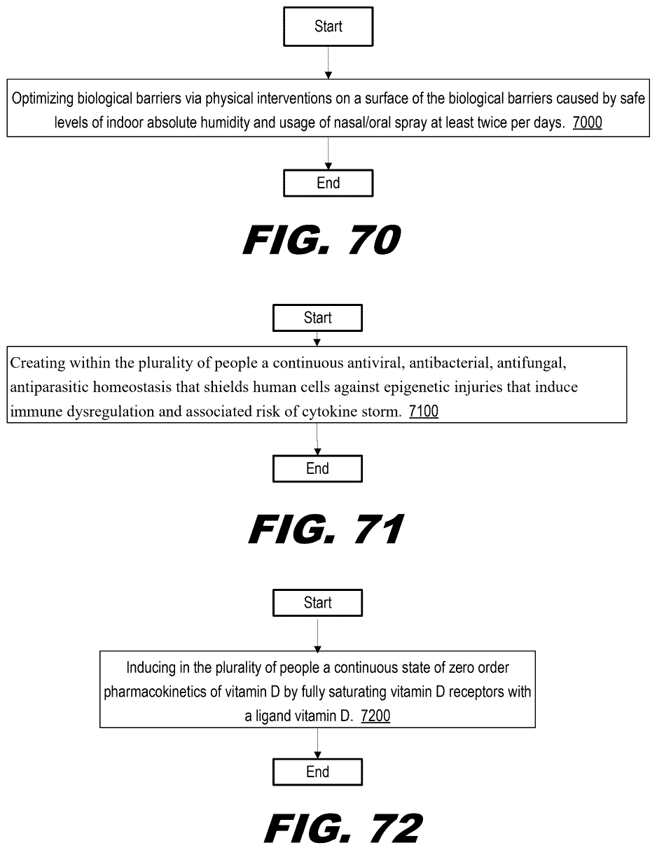

FIG. 70 depicts a process of optimizing biological barriers via physical interventions on a surface of the biological barriers caused by safe levels of indoor absolute humidity and usage of nasal/oral spray at least twice per days 7000. These are illustrative examples of mucosal surfaces in a respiratory tract of people in step 210 in FIG. 2. The process thereafter ends.

FIG. 71 depicts a process of creating within the plurality of people a continuous antiviral, antibacterial, antifungal, antiparasitic homeostasis that shields human cells against epigenetic injuries which induce immune dysregulation and associated risk of cytokine storm 7100. These are illustrative examples of mucosal surfaces in a respiratory tract of people in step 4700 in FIG. 47. The process thereafter ends.

FIG. 72 depicts a process of inducing in the plurality of people a continuous state of zero-order pharmacokinetics of vitamin D by fully saturating vitamin D receptors with a ligand vitamin D 7200. These are illustrative examples of obtaining immunological fitness in step 102 in FIG. 1. The process thereafter ends.

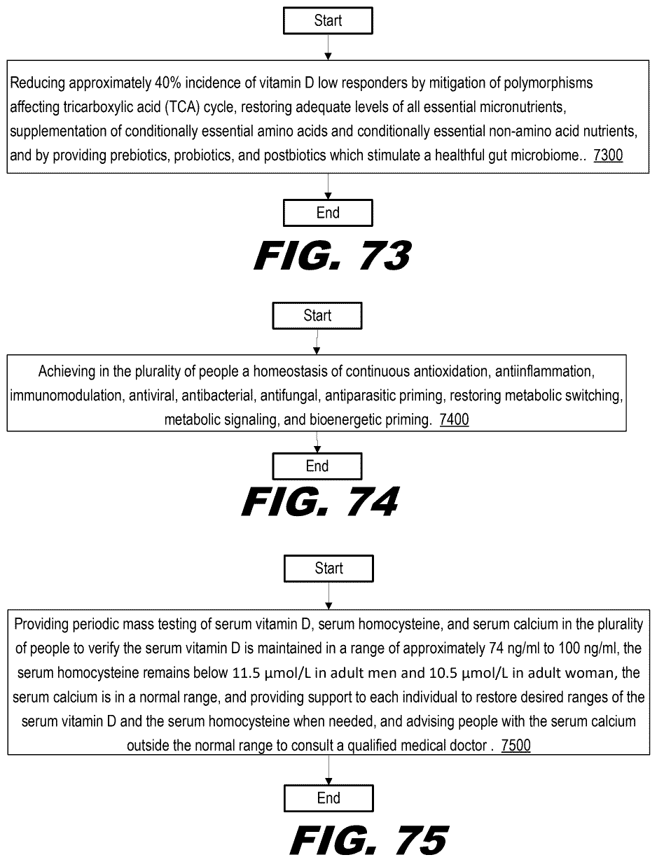

FIG. 73 depicts a process of reducing approximately 40% incidence of vitamin D low responders by mitigation of polymorphisms affecting tricarboxylic acid (TCA) cycle, restoring adequate levels of all essential micronutrients, supplementation of conditionally essential amino acids and conditionally essential non-amino acid nutrients, and by providing prebiotics, probiotics, and postbiotics which stimulate a healthful gut microbiome 7300. These are illustrative examples of obtaining immunological fitness in step 102 in FIG. 1. The process thereafter ends.

FIG. 74 depicts a process of achieving in the plurality of people a homeostasis of continuous antioxidation, anti-inflammation, immunomodulation, antiviral, antibacterial, antifungal, antiparasitic priming, restoring metabolic switching, metabolic signaling, and bioenergetic priming 7400. These are illustrative examples of obtaining immunological fitness in step 102 in FIG. 1. The process thereafter ends.

FIG. 75 depicts a process of providing periodic mass testing of serum vitamin D, serum homocysteine, and serum calcium in the plurality of people to verify the serum vitamin D is maintained in a range of approximately 65 ng/mL to 100 ng/mL, the serum homocysteine remains below 11.5 μmol/L in adult men and 10.5 μmol/L in adult woman, the serum calcium is in a normal range, and providing support to each individual to restore desired ranges of the serum vitamin D and the serum homocysteine when needed, and advising people with the serum calcium outside the normal range to consult a qualified medical doctor 7500. These are illustrative examples of periodic mass testing in step 1384 in FIG. 13. The process thereafter ends.

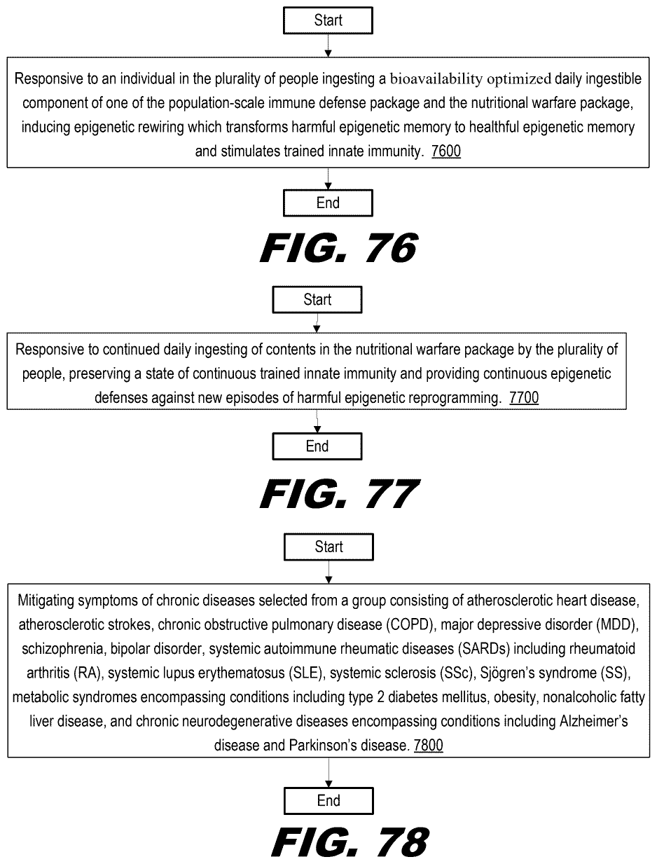

FIG. 76 depicts a process of inducing epigenetic rewiring which transforms harmful epigenetic memory to healthful epigenetic memory and stimulates trained innate immunity which is responsive to an individual in the plurality of people ingesting a bioavailability optimized daily ingestible component of one of the population-scale immune defense package and the nutritional warfare package 7600. These are illustrative examples of transforming harmful epigenetic reprogramming to healthful epigenetic reprogramming in step 110 in FIG. 1. The process thereafter ends.

FIG. 77 depicts a process of preserving a state of continuous trained innate immunity and providing continuous epigenetic defenses against new episodes of harmful epigenetic reprogramming, responsive to continued daily ingesting of contents in the nutritional warfare package by the plurality of people 7700. These are illustrative examples of transforming harmful epigenetic reprogramming to healthful epigenetic reprogramming in step 110 in FIG. 1. The process thereafter ends.

FIG. 78 depicts a process of mitigating symptoms of health problems selected from a group consisting of atherosclerotic heart disease, atherosclerotic strokes, chronic obstructive pulmonary disease (COPD), major depressive disorder (MDD), schizophrenia, bipolar disorder, systemic autoimmune rheumatic diseases (SARDs) including rheumatoid arthritis (RA), systemic lupus erythematosus (SLE), systemic sclerosis (SSc), Sjögren's syndrome (SS), metabolic syndromes encompassing conditions including type 2 diabetes mellitus, obesity, nonalcoholic fatty liver disease, and chronic neurodegenerative diseases encompassing conditions including Alzheimer's disease and Parkinson's disease 7800. These are illustrative examples of transforming harmful epigenetic reprogramming to healthful epigenetic reprogramming in step 110 in FIG. 1. The process thereafter ends.

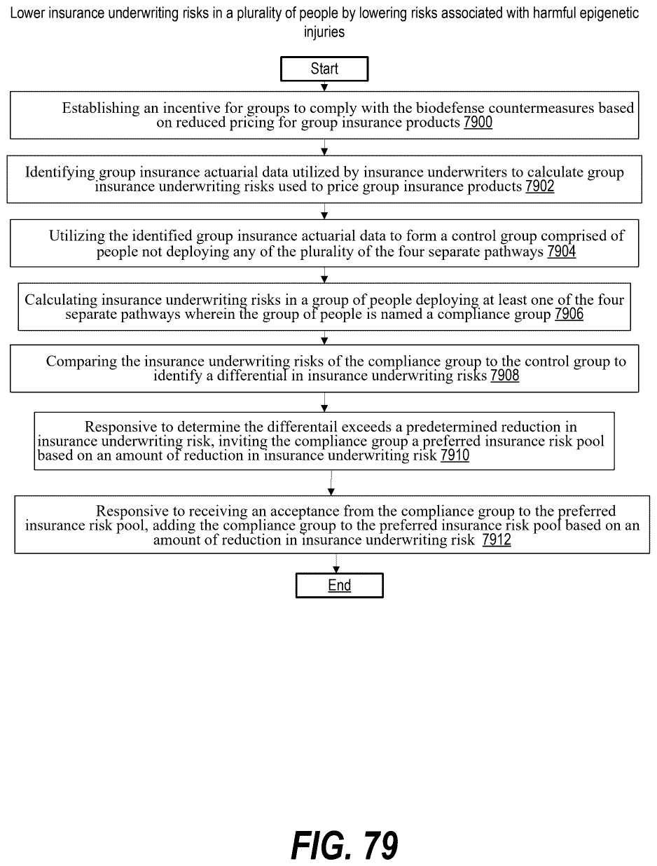

FIG. 79 depicts a process for lowering insurance underwriting risks in a plurality of people resulting in lower prices for group insurance products. In an embodiment, communities that implement biodefense countermeasures against epigenetic warfare automatically mitigates the risks of health problems associated with harmful epigenetic reprogramming. The process utilizes group insurance product discounts as an incentive for groups to comply with biodefense countermeasures (step 7900). The process identifies group insurance actuarial data utilized by insurance underwriters to calculate group insurance underwriting risks used to price group insurance products (step 7902). The process utilizes the identified group insurance actuarial data to form a control group comprised of people not deploying any of the plurality of the four separate pathways (step 7904). The process calculates insurance underwriting risks in a group of people deploying at least one of the four separate pathways wherein the group of people is named a compliance group (step 7906). The process compares the insurance underwriting risks of the compliance group to the control group to identify a mathematical differential in insurance underwriting risks (step 7908). The differential may, for example, be expressed as a percentage of reduction in the insurance underwriting risk in the compliance group. Based up the percentage of reduction, the compliance group may be invited to a preferred insurance risk pool based on the percentage of reduction of insurance underwriting risks. When the compliance groups accept the invitation, the process adds the compliance group to the preferred insurance risk pool (step 7910). These are illustrative examples of transforming harmful epigenetic reprogramming to healthful epigenetic reprogramming in step 110 in FIG. 1. The process thereafter ends.

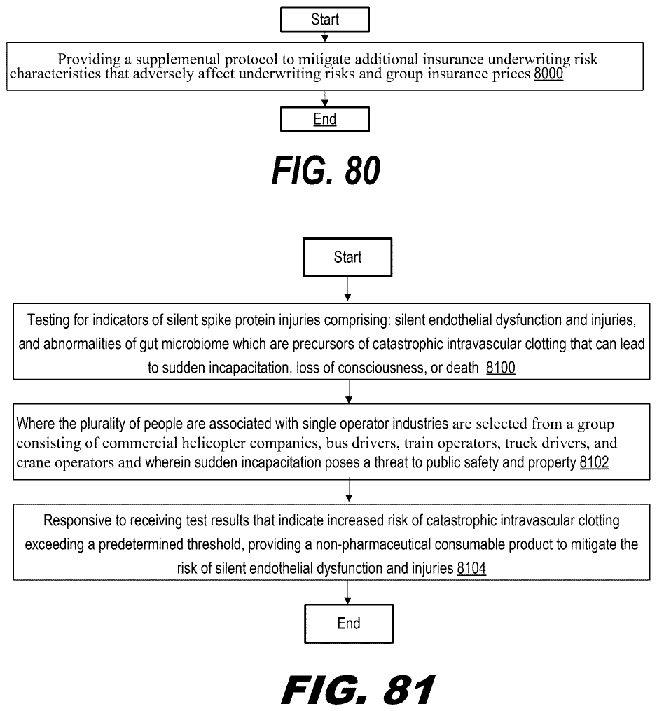

FIG. 80 depicts a process that deploys a supplemental protocol that mitigates additional insurance underwriting risks that adversely affect group insurance prices 8000. In an example embodiment, an industry specific protocol for meat packing plants may verify that the meat packing plant's HVAC system maintains indoor absolute humidity in a viral safe range while still maintaining the cold indoor temperatures required to safely operate the meat packing plant.

FIG. 81 depicts a process which tests for indicators of silent spike protein injuries comprising: silent endothelial dysfunction and injuries, and abnormalities of gut microbiome which are precursors of catastrophic intravascular clotting that can lead to sudden incapacitation, loss of consciousness, or death (step 8100). Where the plurality of people are associated with single operator industries are selected from a group consisting of commercial helicopter companies, bus drivers, train operators, truck drivers, and crane operators and wherein sudden incapacitation poses a threat to public safety and property 8102. Responsive to receiving test results that indicate increased risk of catastrophic intravascular clotting exceeding a predetermined threshold, providing a non-pharmaceutical consumable product to mitigate the risk of silent endothelial dysfunction and injuries (step 8104). In an example embodiment, an extended-release capsule to help mitigate a risk of intravascular oxidative stress over an extended period of time may be supplied to the plurality of people as a supplemental protocol.

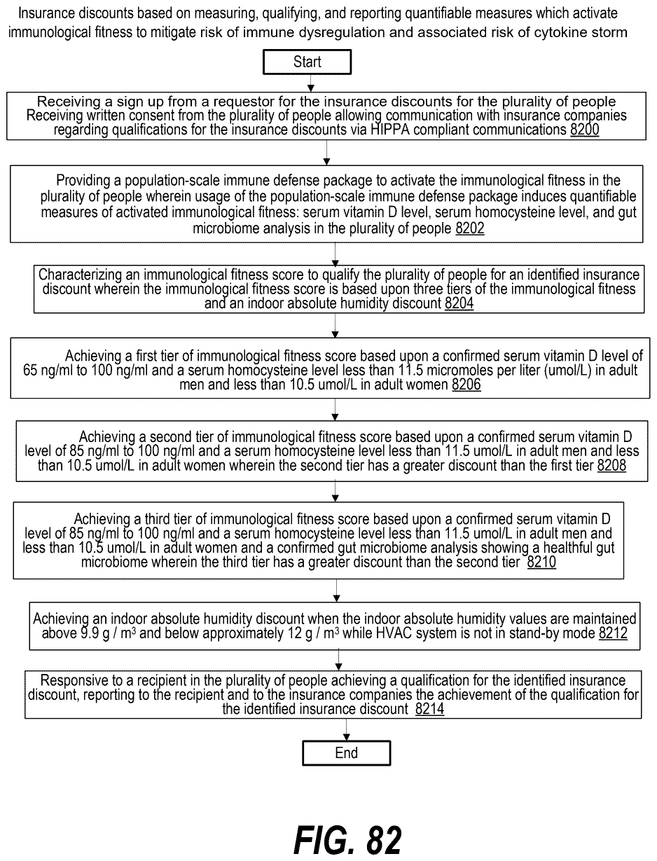

FIG. 82 depicts a process for providing insurance discounts based on measuring, qualifying, and reporting quantifiable measures which activate immunological fitness to mitigate risk of immune dysregulation and associated risk of cytokine storm. The process begins by receiving a sign-up from a requestor for the insurance discounts for the plurality of people. The process then receives written consent from the plurality of people allowing communication with insurance companies regarding qualifications for the insurance discounts via HIPAA compliant communications (step 8200). The process provides a population-scale immune defense package to activate the immunological fitness pathway in the plurality of people wherein usage of the population-scale immune defense package induces quantifiable measures which confirm activation of immunological fitness: serum vitamin D level, serum homocysteine level, and gut microbiome analysis in the plurality of people and activates indoor viral respiratory risk mitigation pathway to optimize biological barriers via physical intervention on a surface of the biological barriers caused by safe levels of indoor absolute humidity where the plurality of people reside (step 8202). The process characterizes an immunological fitness score to qualify the plurality of people for an identified insurance discount wherein the immunological fitness score is based upon three tiers of the immunological fitness and an indoor absolute humidity discount (step 8204). The process achieves a first tier immunological fitness score based upon a confirmed serum vitamin D level of 65 ng/mL to 100 ng/mL and a serum homocysteine level less than 11.5 micromoles per liter (μmol/L) in adult men and less than 10.5 μmol/L in adult women (step 8206). The process achieves a second tier immunological fitness score based upon a confirmed serum vitamin D level of 85 ng/mL to 100 ng/mL and a serum homocysteine level less than 11.5 μmol/L in adult men and less than 10.5 μmol/L in adult women wherein the second tier has a greater discount than the first tier (step 8208). The process achieves a third tier immunological fitness score based upon a confirmed serum vitamin D level of 85 ng/mL to 100 ng/mL and a serum homocysteine level less than 11.5 μmol/Lin adult men and less than 10.5 μmol/L in adult women and a confirmed gut microbiome analysis showing a healthful gut microbiome wherein the third tier has a greater discount than the second tier (step 8210). The process achieves an indoor absolute humidity discount when the indoor absolute humidity values are maintained above 9.9 g/m³ and below approximately 12 g/m³ while HVAC system is not in standby mode (step 8212). The process achieves a qualification for the identified insurance discount, responsive to a recipient in the plurality of people achieving a qualification for the identified insurance discount. The process reports to the recipient and to the insurance companies the achievement of the qualification for the identified insurance discount (step 8214). The process thereafter ends.

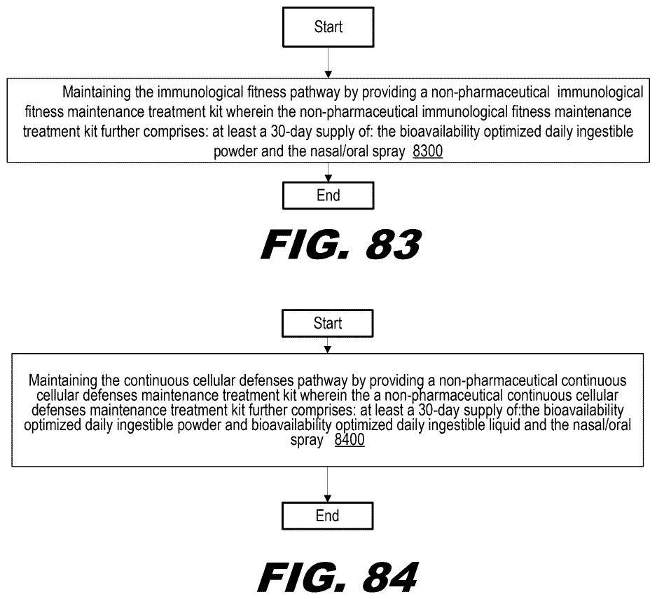

FIG. 83 depicts a process for maintaining the immunological fitness pathway by providing a non-pharmaceutical immunological fitness maintenance treatment kit wherein the non-pharmaceutical immunological fitness maintenance treatment kit further comprises: at least a 30-day supply of: the bioavailability optimized daily ingestible powder and the nasal/oral spray (step 8300).

FIG. 84 depicts a process for maintaining the continuous cellular defenses pathway by providing a non-pharmaceutical continuous cellular defenses maintenance treatment kit wherein the non-pharmaceutical continuous cellular defenses maintenance treatment kit further comprises: at least a 30-day supply of: the bioavailability optimized daily ingestible powder and bioavailability optimized daily ingestible liquid and the nasal/oral spray (step 8400).

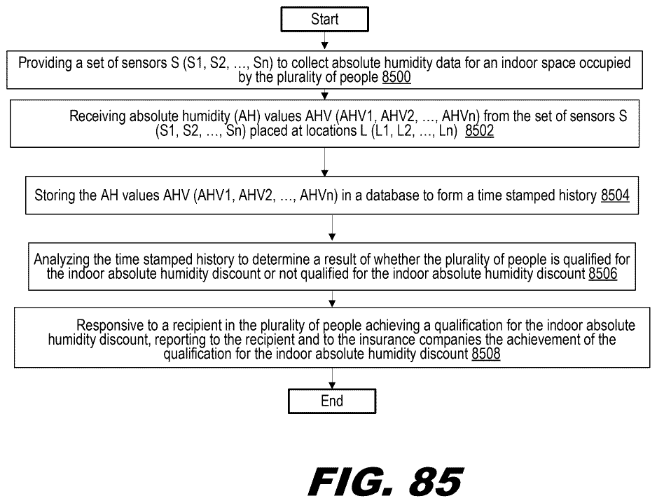

FIG. 85 depicts a process for providing a set of sensors S (S1, S2, . . . , Sn) to collect absolute humidity data for an indoor space occupied by the plurality of people (step 8500). The process receives absolute humidity (AH) values AHV (AHV1, AHV2, . . . , AHVn) from the set of sensors S (S1, S2, . . . , Sn) placed at locations L (L1, L2, . . . , Ln) (step 8502). The process stores the AH values AHV (AHV1, AHV2, . . . , AHVn) in a database to form a time stamped history (step 8504). The process analyzes the time stamped history to determine a result of whether the plurality of people is qualified for the indoor absolute humidity discount or not qualified for the indoor absolute humidity discount (step 8506). The process reports to the recipient and to the insurance companies the achievement of the qualification for the indoor absolute humidity discount, responsive to a recipient in the plurality of people achieving a qualification for the indoor absolute humidity discount (step 8508).

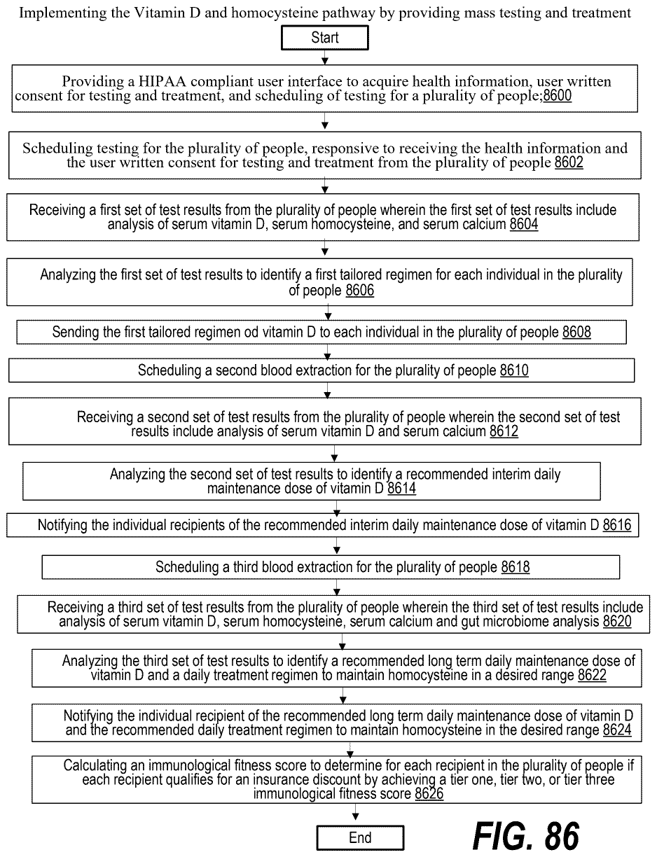

FIG. 86 depicts a process for implementing the vitamin D and homocysteine pathway by providing mass testing and treatment. The process provides a HIPAA compliant user interface to acquire health information, user written consent for testing and treatment, and scheduling of testing for a plurality of people (step 8600). The process schedules testing for the plurality of people responsive to receiving the health information, the user written consent for testing and treatment from the plurality of people (step 8602). The process receives a first set of test results from the plurality of people where the first set of test results include analysis of serum vitamin D, serum homocysteine, and serum calcium (step 8604). The process analyzes the first set of test results to identify a first tailored regimen for each individual in the plurality of people (step 8606). The process sends the first tailored regimen of vitamin D to each individual in the plurality of people (step 8608). The process schedules a second blood extraction for the plurality of people (step 8610). The process receives a second set of test results from the plurality of people where the second set of test results include analysis of serum vitamin D and serum calcium (step 8612). The process analyzes the second set of test results to identify a recommended interim daily maintenance dose of vitamin D (step 8614). The process notifies the individual recipients of the recommended interim daily maintenance dose of vitamin D (step 8616). The process schedules a third blood extraction and a gut microbiome analysis for the plurality of people (step 8618). The process receives a third set of test results from the plurality of people wherein the third set of test results include analysis of serum vitamin D, serum homocysteine, serum calcium and gut microbiome analysis (step 8620). The process analyzes the third set of test results to identify a recommended long term daily maintenance dose of vitamin D and a daily treatment regimen to maintain homocysteine in a desired range (step 8622). The process notifies the individual recipient of the recommended long term daily maintenance dose of vitamin D and the recommended daily treatment regimen to maintain homocysteine in the desired range (step 8624). The process calculates the immunological fitness score to determine for each recipient in the plurality of people if each recipient qualifies for an insurance discount by achieving a tier one, tier two, or tier three immunological fitness score (step 8626).

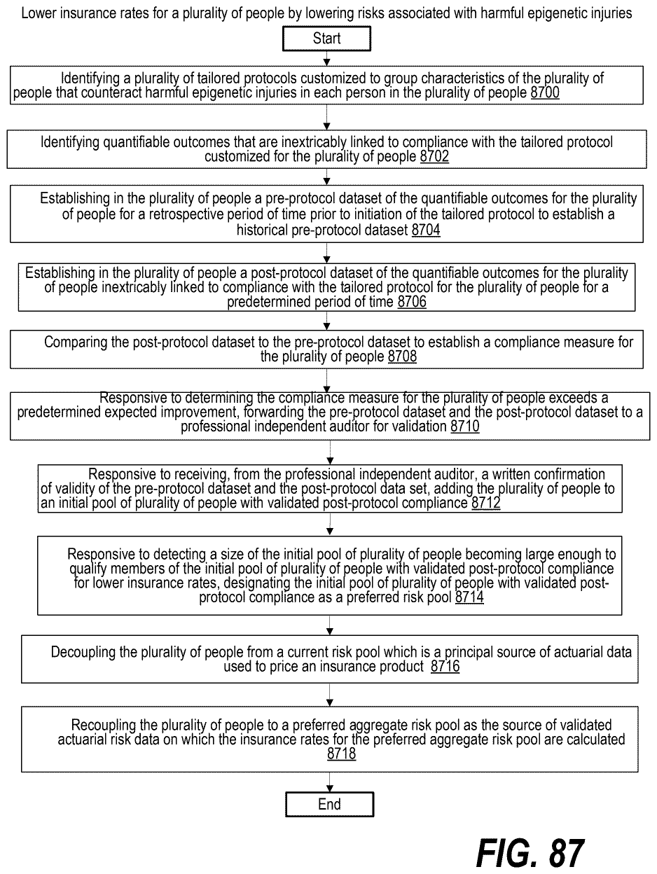

FIG. 87 depicts steps for a process to lower insurance rates for plurality of people by lowering risk in the plurality of people associated with harmful epigenetic injuries.

The process identifies a plurality of tailored protocols customized to group characteristics of the plurality of people that counteract harmful epigenetic injuries in each plurality of people (step 8700).

The process identifies quantifiable outcomes that are inextricably linked to compliance with the tailored protocol customized for each plurality of people (step 8702).

The process establishes, in each plurality of people, a pre-protocol dataset of the quantifiable outcomes for the plurality of people for a retrospective period of time prior to initiation of the tailored protocol to establish a historical pre-protocol dataset (step 8704).

The process establishes, in each plurality of people, a post-protocol dataset of the quantifiable outcomes for the plurality of people inextricably linked to compliance with the tailored protocol for the population for a predetermined period of time (step 8706).

The process compares the post-protocol dataset to the pre-protocol dataset to establish a compliance measure for the plurality of people (step 8708).

The process forwards the pre-protocol dataset and the post-protocol dataset to a professional independent auditor for validation, responsive to determining the compliance measure for the plurality of people exceeds a predetermined expected improvement (step 8710).

The process adds the plurality of people to an initial pool of plurality of people with validated post-protocol compliance, responsive to receiving, from the professional independent auditor, a written confirmation of validity of the pre-protocol dataset and the post-protocol data set (step 8712).

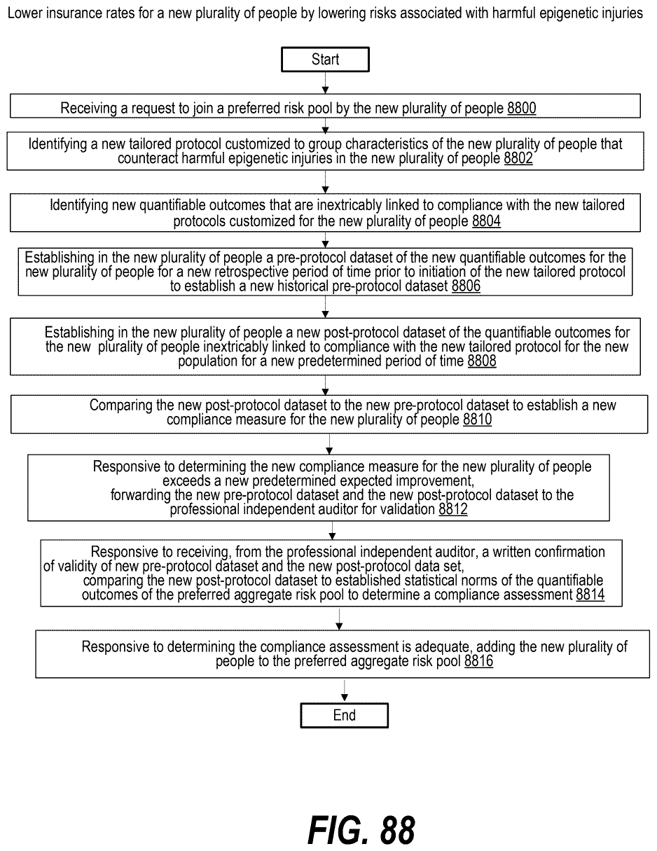

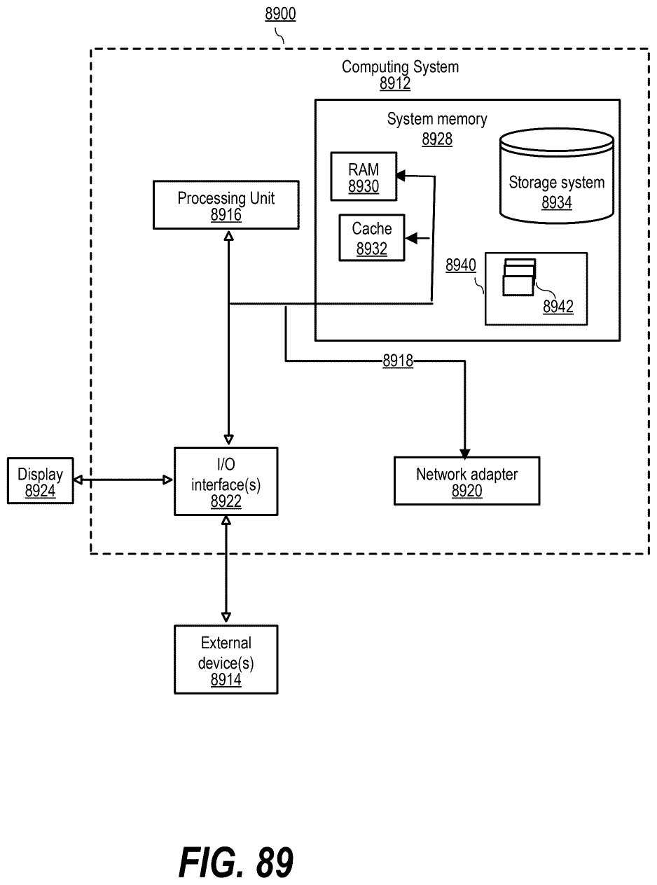

FIG. 88 depicts steps for a process to lower insurance rates for a new plurality of people by lowering risk in the new plurality of people associated with harmful epigenetic injuries. The process receives a request from the new plurality of people to join the preferred aggregate risk pool (step 8800). The process identifies new tailored protocols customized to group characteristics of the new plurality of people that counteract harmful epigenetic injuries in the new plurality of people (step 8802). The process identifies new quantifiable outcomes that are inextricably linked to compliance with the new tailored protocols customized for the new plurality of′ people (step 8804). The process establishes in the new plurality of people a pre-protocol dataset of the new quantifiable outcomes for the new plurality of people for a new retrospective period of time prior to initiation of the new tailored protocol to establish a new historical pre-protocol dataset (step 8806). The process establishes in the new plurality of people a new post-protocol dataset of the quantifiable outcomes for the new plurality of people inextricably linked to compliance with the new tailored protocol for the new population for a new predetermined period of time (step 8808). The process compares the new post-protocol dataset to the new pre-protocol dataset to establish a new compliance measure for the new plurality of people (step 8810). The process forwards the new pre-protocol dataset and the new post-protocol dataset to the professional independent auditor for validation, responsive to determining the new compliance measure for the new plurality of people exceeds a new predetermined expected improvement (step 8812). The process compares the new post-protocol dataset to established statistical norms of the quantifiable outcomes of the preferred aggregate risk pool to determine a compliance assessment, responsive to receiving, from the professional independent auditor, a written confirmation of validity of new pre-protocol dataset and the new post-protocol data set (step 8814). The process adds the new plurality of people to the preferred aggregate risk pool, responsive to determining the compliance assessment is adequate (step 8816).

Referring to FIG. 80, a schematic view of a processing system 8000 is shown wherein the methods of this invention may be implemented. The processing system 8000 is only one example of a suitable system and is not intended to suggest any limitation as to the scope of use or functionality of embodiments of the invention described herein. Regardless, the system 8000 can implement and/or perform any of the functionality set forth herein. In the system 8000 there is a computer system 8012, which is operational with numerous other general purpose or special purpose computing system environments or configurations. Examples of well-known computing systems, environments, and/or configurations that may be suitable for use with the computer system 8012 include, but are not limited to, personal computer systems, server computer systems, thin clients, thick clients, handheld or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

The computer system 8012 may be described in the general context of computer system-executable instructions, such as program modules, being executed by a computer system. Generally, program modules may include routines, programs, objects, components, logic, data structures, and so on that perform tasks or implement abstract data types. The computer system 8012 may be practiced in distributed cloud computing environments where tasks are performed by remote processing devices that are linked through a communications network. In a distributed cloud computing environment, program modules may be in both local and remote computer system storage media including memory storage devices.

As shown in FIG. 80, the computer system 8012 in the system environment 8000 is shown in the form of a general-purpose computing device. The components of the computer system 8012 may include, but are not limited to, a set of one or more processors or processing units 8016, a system memory 8028, and a bus 8018 that couples various system components including the system memory 8028 to the processor 8016.

The bus 8018 represents one or more of any of several types of bus structures, including a memory bus or memory controller, a peripheral bus, an accelerated graphics port, and a processor or local bus using any of a variety of bus architectures. By way of example, and not limitation, such architectures include the Industry Standard Architecture (ISA) bus, the Micro Channel Architecture (MCA) bus, the Enhanced ISA (EISA) bus, the Video Electronics Standards Association (VESA) local bus, and the Peripheral Component Interconnects (PCI) bus.

The computer system 8012 typically includes a variety of computer system readable media. Such media may be any available media that is accessible by the computer system 8012, and it includes both volatile and non-volatile media, removable and non-removable media.

The system memory 8028 can include computer system readable media in the form of volatile memory, such as random-access memory (RAM) 8030 and/or a cache memory 8032. The computer system 8012 may further include other removable/non-removable, volatile/non-volatile computer system storage media. By way of example only, a storage system 8034 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to the bus 8018 by one or more data media interfaces. As will be further depicted and described below, the system memory 8028 may include at least one program product having a set (e.g., at least one) of program modules 8042 that are configured to carry out the functions of embodiments of the invention.

A program/utility 8040, having the set (at least one) of program modules 8042, may be stored in the system memory 8028 by way of example, and not limitation, as well as an operating system, one or more application programs, other program modules, and program data. Each of the operating systems may have one or more application programs, other program modules, and program data or some combination thereof, and may include an implementation of a networking environment. The program modules 8042 generally carry out the functions and/or methodologies of embodiments of the invention as described herein.

The computer system 8012 may also communicate with a set of one or more external devices 8014 such as a keyboard, a pointing device, a display 8024, a tablet, a digital pen, etc. wherein these one or more devices enable a user to interact with the computer system 8012; and/or any devices (e.g., network card, modem, etc.) that enable the computer system 8012 to communicate with one or more other computing devices. Such communication can occur via Input/Output (I/O) interfaces 8022. These include wireless devices and other devices that may be connected to the computer system 8012, such as, a USB port, which may be used by a tablet device (not shown). Still yet, the computer system 8012 can communicate with one or more networks such as a local area network (LAN), a general wide area network (WAN), and/or a public network (e.g., the Internet) via a network adapter 8020. As depicted, a network adapter 8020 communicates with the other components of the computer system 8012 via the bus 8018. It should be understood that although not shown, other hardware and/or software components could be used in conjunction with the computer system 8012. Examples include, but are not limited to microcode, device drivers, redundant processing units, external disk drive arrays, RAID systems, tape drives, and data archival storage systems, etc.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

While particular embodiments have been shown and described, it will be obvious to those skilled in the art that, based upon the teachings herein, that changes and modifications may be made without departing from this invention and its broader aspects. Therefore, the appended claims are to encompass within their scope all such changes and modifications as are within the true spirit and scope of this invention. Furthermore, it is to be understood that the invention is solely defined by the appended claims. It will be understood by those with skill in the art that if a specific number of an introduced claim element is intended, such intent will be explicitly recited in the claim, and in the absence of such recitation no such limitation is present. For non-limiting example, as an aid to understanding, the following appended claims contain usage of the introductory phrases “at least one” and “one or more” to introduce claim elements. However, the use of such phrases should not be construed to imply that the introduction of a claim element by the indefinite articles “a” or “an” limits any particular claim containing such introduced claim element to inventions containing only one such element, even when the same claim includes the introductory phrases “one or more” or “at least one” and indefinite articles such as “a” or “an”; the same holds true for the use in the claims of definite articles.

What is claimed is:

1. A non-pharmaceutical immunological fitness and continuous cellular defenses kit, comprising:
a bioavailability-optimized ingestible powder comprising prebiotics, probiotics, postbiotics, essential micronutrients, bioavailability-optimized phytochemicals, microRNAs, and amino acids;
a bioavailability-optimized ingestible liquid comprising non-pharmaceutical phytochemicals and essential micronutrients, wherein the non-pharmaceutical phytochemicals are encapsulated using food-grade nanotechnology to improve bioavailability; and
instructions provided with the kit associating coordinated use of the ingestible powder and the ingestible liquid with activation of at least one biological defense pathway selected from a vitamin D and homocysteine pathway, an immunological fitness pathway, and a continuous cellular defenses pathway,
wherein the kit, as packaged and distributed, is configured for population-scale deployment to mitigate immune dysregulation and epigenetic injury without requiring pharmaceutical intervention.

2. The kit of claim 1, further comprising an immediate treatment kit component configured for short-term use in response to early signs of infectious illness.

3. The kit of claim 1, wherein the kit is configured as a maintenance kit comprising a supply of the ingestible powder and the ingestible liquid for repeated daily use over a predetermined maintenance period.

4. The kit of claim 1, wherein the ingestible powder and the ingestible liquid are formulated according to demographic classifications selected from adults, children, military personnel, first responders, or institutional populations.

5. The kit of claim 1, wherein components of the kit are functionally interdependent and lack substantial non-infringing use when distributed together.

6. The kit of claim 1, wherein the instructions, labeling, or electronic content expressly direct coordinated use of the ingestible powder and the ingestible liquid to activate the at least one biological defense pathway.

7. The kit of claim 1, wherein the kit is marketed, labeled, or configured to mitigate actuarial risk associated with immune dysregulation and epigenetic injury.

8. The kit of claim 1, wherein removal or substitution of any one component materially degrades an intended immunological risk-mitigation functionality of the kit.

9. A non-pharmaceutical immunological preparedness kit, comprising:
a plurality of ingestible compositions packaged together, including at least one bioavailability-optimized powder and at least one bioavailability-optimized liquid;
an interim vitamin D dosage component packaged for administration prior to individualized serum vitamin D testing; and
guidance materials provided with the kit associating use of the ingestible compositions with immunological fitness qualification metrics,
wherein the kit is configured for population-scale preparedness against immune dysregulation independent of user-performed medical treatment.

10. The kit of claim 9, wherein the interim vitamin D dosage component is configured for administration for a predetermined duration without serum vitamin D testing.

11. The kit of claim 9, wherein the guidance materials associate kit usage with target serum vitamin D and serum homocysteine ranges.

12. The kit of claim 9, wherein the ingestible compositions are packaged for distribution to institutional, occupational, or community populations.

13. The kit of claim 9, wherein the plurality of ingestible compositions is structured and promoted as a unified immunological defense solution.

14. The kit of claim 9, wherein removal or substitution of any one of the ingestible compositions materially degrades an intended immunological risk-mitigation functionality of the kit.

15. A system for population-scale immunological risk mitigation, comprising:
a plurality of non-pharmaceutical immunological fitness kits, each kit comprising at least one ingestible powder and at least one ingestible liquid configured to support immunological fitness;
a distribution mechanism configured to deliver the plurality of kits to a population group;
a data association component configured to associate kit distribution or usage with immunological fitness metrics; and
a processing component configured to determine compliance status, qualification status, or population-level risk metrics based on the immunological fitness metrics.

16. The system of claim 15, further comprising a plurality of environmental sensors configured to measure indoor absolute humidity and associate measured indoor absolute humidity values with biological barrier optimization metrics.

17. The system of claim 15, wherein deployment of the kits without the data association component materially reduces an intended population-scale immunological risk-mitigation benefit.

18. The system of claim 15, wherein the system is offered, licensed, or deployed with knowledge that distribution of the kits will result in use consistent with predefined immunological defense pathways.

19. A method for immunological fitness and continuous cellular defenses, comprising:
activating at least one of four pathways to induce components of immunological fitness and continuous cellular defenses in a plurality of people,
wherein the four pathways comprise a vitamin D and homocysteine pathway, an indoor viral respiratory risk mitigation pathway, an immunological fitness pathway, and a continuous cellular defenses pathway;
achieving zero-order pharmacokinetics of vitamin D and maintaining serum homocysteine below a predetermined threshold;
optimizing biological barriers against airborne pathogens through maintenance of viral-safe indoor absolute humidity;
administering non-pharmaceutical nutritional compositions to induce immunological fitness and continuous cellular defenses; and
mitigating risk of immune dysregulation, cytokine storm, and epigenetic injury based on activation of the at least one pathway.

20. The method of claim 19, wherein administering non-pharmaceutical nutritional compositions comprises coordinated administration of a bioavailability-optimized powder and a bioavailability-optimized liquid.

21. The method of claim 19, further comprising determining vitamin D dosing based on serum testing results.

22. The method of claim 19, wherein vitamin D is administered for a predetermined interim period without serum testing.

23. The method of claim 19, further comprising inducing epigenetic rewiring that transforms harmful epigenetic memory into trained innate immunity.

24. A method for lowering insurance underwriting risk for a plurality of people, comprising:
deploying one or more non-pharmaceutical immunological fitness kits to the plurality of people;
measuring quantifiable biological outcomes including serum vitamin D level, serum homocysteine level, and biological barrier optimization;
comparing measured biological outcomes to actuarial risk benchmarks;
validating compliance through an independent assessment; and
generating and providing underwriting-relevant risk reduction data or rate-adjustment recommendations to one or more third-party insurance underwriters based on validated reduction data.

25. The method of claim 24, wherein the underwriting-relevant data comprises a recommended premium modifier, risk class adjustment, or eligibility qualification.

26. The method of claim 24, further comprising electronically interfacing with a third-party underwriting system to transmit the validated biological risk reduction data.

27. The method of claim 24, wherein the underwriting-relevant data is updated dynamically based on subsequent biological outcome measurements.
